SEQUENCE LISTING (19 sequences)

SEQ ID NO: 1 - protein, 2785 aa, Homo sapiens
MKTKPVSHKTENTYRFLTFAERLGNVNIDIIHRIDRTASYEEEVETYFFEGLLKWRELNLTEHFGKFYKEVIDKCQSFNQLVYHQNEIVQSLKTHLQVKNSFAYQPLLDLVVQLARDLQMDFYPHFPEFFLTITSILETQDTELLEWAFTSLSYLYKYLWRLMVKDMSSIYSMYSTLLAHKKLHIRNFAAESFTFLMRKVSDKNALFNLMFLDLDKHPEKVEGVGQLLFEMCKGVRNMFHSCTGQAVKLILRKLGPVTETETQLPWMLIGETLKNMVKSTVSYISKEHFGTFFECLQESLLDLHTKVTKTNCCESSEQIKRLLETYLILVKHGSGTKIPTPADVCKVLSQTLQVASLSTSCWETLLDVISALILGENVSLPETLIKETIEKIFESRFEKRLIFSFSEVMFAMKQFEQLFLPSFLSYIVNCFLIDDAVVKDEALAILAKLILNKAAPPTAGSMAIEKYPLVFSPQMVGFYIKQKKTRSKGRNEQFPVLDHLLSIIKLPPNKDTTYLSQSWAALVVLPHIRPLEKEKVIPLVTGFIEALFMTVDKGSFGKGNLFVLCQAVNTLLSLEESSELLHLVPVERVKNLVLTFPLEPSVLLLTDLYYQRLALCGCKGPLSQEALMELFPKLQANISTGVSKIRLLTIRILNHFDVQLPESMEDDGLSERQSVFAILRQAELVPATVNDYREKLLHLRKLRHDVVQTAVPDGPLQEVPLRYLLGMLYINFSALWDPVIELISSHAHEMENKQFWKVYYEHLEKAATHAEKELQNDMTDEKSVGDESWEQTQEGDVGALYHEQLALKTDCQERLDHTNFRFLLWRALTKFPERVEPRSRELSPLFLRFINNEYYPADLQVAPTQDLRRKGKGMVAEEIEEEPAAGDDEELEEEAVPQDESSQKKKTRRAAAKQLIAHLQVFSKFSNPRALYLESKLYELYLQLLLHQDQMVQKITLDCIMTYKHPHVLPYRENLQRLLEDRSFKEEIVHFSISEDNAVVKTAHRADLFPILMRILYGRMKNKTGSKTQGKSASGTRMAIVLRFLAGTQPEEIQIFLDLLFEPVRHFKNGECHSAVIQAVEDLDLSKVLPLGRQHGILNSLEIVLKNISHLISAYLPKILQILLCMTATVSHILDQREKIQLRFINPLKNLRRLGIKMVTDIFLDWESYQFRTEEIDAVFHGAVWPQISRLGSESQYSPTPLLKLISIWSRNARYFPLLAKQKPGHPECDILTNVFAILSAKNLSDATASIVMDIVDDLLNLPDFEPTETVLNLLVTGCVYPGIAENIGESITIGGRLILPHVPAILQYLSKTTISAEKVKKKKNRAQVSKELGILSKISKFMKDKEQSSVLITLLLPFLHRGNIAEDTEVDILVTVQNLLKHCVDPTSFLKPIAKLFSVIKNKLSRKLLCTVFETLSDFESGLKYITDVVKLNAFDQRHLDDINFDVRFETFQTITSYIKEMQIVDVNYLIPVMHNCFYNLELGDMSLSDNASMCLMSIIKKLPALNVTEKDYREIIHRSLLEKLRKGLKSQTESIQQDYTTILSCLIQTFPNQLEFKDLVQLTHYHDPEMDFFENMKHIQIHRRARALKKLAKQLMEGKVVLSSKSLQNYIMPYAMTPIFDEKMLKHENITTAATEIIGAICKHLSWSAYMYYLKHFIHVLQTGQINQKLGVSLLVIVLEAFHFDHKTLEEQMGKIENEENAIEAIELPEPEAMELERVDEEEKEYTCKSLSDNGQPGTPDPADSGGTSAKESECITKPVSFLPQNKEEIERTIKNIQGTITGDILPRLHKCLASTTKREEEHKLVKSKVVNDEEVVRVPLAFAMVKLMQSLPQEVMEANLPSILLKVCALLKNRAQEIRDIARSTLAKIIEDLGVHFLQYVLKELQTTLVRGYQVHVLTFTVHMLLQGLTNKLQVGDLDSCLDIMIEIFNHELFGAVAEEKEVKQILSKVMEARRSKSYDSYEILGKFVGKDQVTKLILPLKEILQNTTSLKLARKVHETLRRITVGLIVNQEMTAESILLLSYGLISENLPLLTEKEKNPVAPAPDPRLPPQSCLLLPPTPVRGGQKAVVSRKTNMHIFIESGLRLLHLSLKTSKIKSSGECVLEMLDPFVSLLIDCLGSMDVKVITGALQCLIWVLRFPLPSIETKAEQLTKHLFLLLKDYAKLGAARGQNFHLVVNCFKCVTILVKKVKSYQITEKQLQVLLAYAEEDIYDTSRQATAFGLLKAILSRKLLVPEIDEVMRKVSKLAVSAQSEPARVQCRQVFLKYILDYPLGDKLRPNLEFMLAQLNYEHETGRESTLEMIAYLFDTFPQGLLHENCGMFFIPLCLMTINDDSATCKKMASMTIKSLLGKISLEKKDWLFDMVTTWFGAKKRLNRQLAALICGLFVESEGVDFEKRLGTVLPVIEKEIDPENFKDIMEETEEKAADRLLFSFLTLITKLIKECNIIQFTKPAETLSKIWSHVHSHLRHPHNWVWLTAAQIFGLLFASCQPEELIQKWNTKKTKKHLPEPVAIKFLASDLDQKMKSISLASCHQLHSKFLDQSLGEQVVKNLLFAAKVLYLLELYCEDKQSKIKEDLEEQEALEDGVACADEKAESDGEEKEEVKEELGRPATLLWLIQKLSRIAKLEAAYSPRNPLKRTCIFKFLGAVAMDLGIDKVKPYLPMIIAPLFRELNSTYSEQDPLLKNLSQEIIELLKKLVGLESFSLAFASVQKQANEKRALRKKRKALEFVTNPDIAAKKKMKKHKNKSEAKKRKIEFLRPGYKAKRQKSHSLKDLAMVE

SEQ ID NO: 2 - protein, 919 aa, Homo sapiens [sequence omitted]

SEQ ID NO: 3 - protein, 610 aa, Homo sapiens
METAPKPGKDVPPKKDKLQTKRKKPRRYWEEETVPTTAGASPGPPRNKKNRELRPQRPKNAYILKKSRISKKPQVPKKPREWKNPESQRGLSGAQDPFPGPAPVPVEVVQKFCRIDKSRKLPHSKAKTRSRLEVAEAEEEETSIKAARSELLLAEEPGFLEGEDGEDTAKICQADIVEAVDIASAAKHFDLNLRQFGPYRLNYSRTGRHLAFGGRRGHVAALDWVTKKLMCEINVMEAVRDIRFLHSEALLAVAQNRWLHIYDNQGIELHCIRRCDRVTRLEFLPFHFLLATASETGFLTYLDVSVGKIVAALNARAGRLDVMSQNPYNAVIHLGHSNGTVSLWSPAMKEPLAKILCHRGGVRAVAVDSTGTYMATSGLDHQLKIFDLRGTYQPLSTRTLPHGAGHLAFSQRGLLVAGMGDVVNIWAGQGKASPPSLEQPYLTHRLSGPVHGLQFCPFEDVLGVGHTGGITSMLVPGAGEPNFDGLESNPYRSRKQRQEWEVKALLEKVPAELICLDPRALAEVDVISLEQGKKEQIERLGYDPQAKAPFQPKPKQKGRSSTASLVKRKRKVMDEEHRDKVRQSLQQQHHKEAKAKPTGARPSALDRFVR

SEQ ID NO: 4 - protein, 556 aa, Homo sapiens
MPPERRRRMKLDRRTGAKPKRKPGMRPDWKAGAGPGGPPQKPAPSSQRKPPARPSAAAAAIAVAAAEEERRLRQRNRLRLEEDKPAVERCLEELVFGDVENDEDALLRRLRGPRVQEHEDSGDSEVENEAKGNFPPQKKPVWVDEEDEDEEMVDMMNNRFRKDMMKNASESKLSKDNLKKRLKEEFQHAMGGVPAWAETTKRKTSSDDESEEDEDDLLQRTGNFISTSTSLPRGILKMKNCQHANAERPTVARISSVQFHPGAQIVMVAGLDNAVSLFQVDGKTNPKIQSIYLERFPIFKACFSANGEEVLATSTHSKVLYVYDMLAGKLIPVHQVRGLKEKIVRSFEVSPDGSFLLINGIAGYLHLLAMKTKELIGSMKINGRVAASTFSSDSKKVYASSGDGEVYVWDVNSRKCLNRFVDEGSLYGLSIATSRNGQYVACGSNCGVVNIYNQDSCLQETNPKPIKAIMNLVTGVTSLTFNPTTEILAIASEKMKEAVRLVHLPSCTVFSNFPVIKNKNISHVHTMDFSPRSGYFALGNEKGKALMYRLHHYSDF

SEQ ID NO: 5 - protein, 681 aa, Homo sapiens
MAPQVWRRRTLERCLTEVGKATGRPECFLTIQEGLASKFTSLTKVLYDFNKILENGRIHGSPLQKLVIENFDDEQIWQQLELQNEPILQYFQNAVSETINDEDISLLPESEEQEREEDGSEIEADDKEDLEDLEEEEVSDMGNDDPEMGERAENSSKSDLRKSPVFSDEDSDLDFDISKLEQQSKVQNKGQGKPREKSIVDDKFFKLSEMEAYLENIEKEEERKDDNDEEEEDIDFFEDIDSDEDEGGLFGSKKLKSGKSSRNLKYKDFFDPVESDEDITNVHDDELDSNKEDDEIAEEEAEELSISETDEDDDLQENEDNKQHKESLKRVTFALPDDAETEDTGVLNVKKNSDEVKSSFEKRQEKMNEKIASLEKELLEKKPWQLQGEVTAQKRPENSLLEETLHFDHAVRMAPVITEETTLQLEDIIKQRIRDQAWDDVVRKEKPKEDAYEYKKRLTLDHEKSKLSLAEIYEQEYIKLNQQKTAEEENPEHVEIQKMMDSLFLKLDALSNFHFIPKPPVPEIKVVSNLPAITMEEVAPVSVSDAALLAPEEIKEKNKAGDIKTAAEKTATDKKRERRKKKYQKRMKIKEKEKRRKLLEKSSVDQAGKYSKTVASEKLKQLTKTGKASFIKDEGKDKALKSSQAFFSKLQDQVKMQINDAKKTEKKKKKRQDISVHKLKL

SEQ ID NO: 6 - protein, 943 aa, Homo sapiens
MGLTKQYLRYVASAVFGVIGSQKGNIVFVTLRGEKGRYVAVPACEHVFIWDLRKGEKILILQGLKQEVTCLCPSPDGLHLAVGYEDGSIRIFSLLSGEGNVTFNGHKAAITTLKYDQLGGRLASGSKDTDIIVWDVINESGLYRLKGHKDAITQALFLREKNLLVTSGKDTMVKWWDLDTQHCFKTMVGHRTEVWGLVLLSEEKRLITGASDSELRVWDIAYLQEIEDPEEPDPKKIKGSSPGIQDTLEAEDGAFETDEAPEDRILSCRKAGSIMREGRDRVVNLAVDKTGRILACHGTDSVLELFCILSKKEIQKKMDKKMKKARKKAKLHSSKGEEEDPEVNVEMSLQDEIQRVTNIKTSAKIKSFDLIHSPHGELKAVFLLQNNLVELYSLNPSLPTPQPVRTSRITIGGHRSDVRTLSFSSDNIAVLSAAADSIKIWNRSTLQCIRTMTCEYALCSFFVPGDRQVVIGTKTGKLQLYDLASGNLLETIDAHDGALWSMSLSPDQRGFVTGGADKSVKFWDFELVKDENSTQKRLSVKQTRTLQLDEDVLCVSYSPNQKLLAVSLLDCTVKIFYVDTLKFFLSLYGHKLPVICMDISHDGALIATGSADRNVKIWGLDFGDCHKSLFAHDDSVMYLQFVPKSHLFFTAGKDHKIKQWDADKFEHIQTLEGHHQEIWCLAVSPSGDYVVSSSHDKSLRLWERTREPLILEEEREMEREAEYEESVAKEDQPAVPGETQGDSYFTGKKTIETVKAAERIMEAIELYREETAKMKEHKAICKAAGKEVPLPSNPILMAYGSISPSAYVLEIFKGIKSSELEESLLVLPFSYVPDILKLFNEFIQLGSDVELICRCLFFLLRIHFGQITSNQMLVPVIEKLRETTISKVSQVRDVIGFNMAGLDYLKRECEAKSEVMFFADATSHLEEKKRKRKKREKLILTLT

SEQ ID NO: 7 - protein, 519 aa, Homo sapiens
MAFDPTSTLLATGGCDGAVRVWDIVRHYGTHHFRGSPGVVHLVAFHPDPTRLLLFSSATDAAIRVWSLQDRSCLAVLTAHYSAVTSLAFSADGHTMLSSGRDKICIIWDLQSCQATRTVPVFESVEAAVLLPEEPVSQLGVKSPGLYFLTAGDQGTLRVWEAASGQCVYTQAQPPGPGQELTHCTLAHTAGVVLTATADHNLLLYEARSLRLQKQFAGYSEEVLDVRFLGPEDSHVVVASNSPCLKVFELQTSACQILHGHTDIVLALDVFRKGWLFASCAKDQSVRIWRMNKAGQVMCVAQGSGHTHSVGTVCCSRLKESFLVTGSQDCTVKLWPLPKALLSKNTAPDNGPILLQAHTTQRCHDKDINSVAIAPNDKLLATGSQDRTAKLWALPQCQLLGVFSGHRVASGASSSLPWTRCWPRPQLMAPSSSGHSRTSAVSRHLRGTMLLCLKVAFVSRGTQLLSSGSDGLVKLWTIKNNECVRTLDAHEDKVWGLQAGWTTTPSLGPVTPESSSGRM

SEQ ID NO: 8 - protein, 951 aa, Homo sapiens
MCCTEGSLRKRDSQRAPEAVLCLQLWQRTVPLDTLKGLGTCFPSGPELRGAGIAAAMERASERRTASALFAGFRALGLFSNDIPHVVRFSALKRRFYVTTCVGKSFHTYDVQKLSLVAVSNSVPQDICCMAADGRLVFAAYGNVFSAFARNKEIVHTFKGHKAEIHFLQPFGDHIISVDTDGILIIWHIYSEEEYLQLTFDKSVFKISAILHPSTYLNKILLGSEQGSLQLWNVKSNKLLYTFPGWKVGVTALQQAPAVDVVAIGLMSGQVIIHNIKFNETLMKFRQDWGPITSISFRTDGHPVMAAGSPCGHIGLWDLEDKKLINQMRNAHSTAIAGLTFLHREPLLVTNGADNALRIWIFDGPTGEGRLLRFRMGHSAPLTNIRYYGQNGQQILSASQDGTLQSFSTVHEKFNKSLGHGLINKKRVKRKGLQNTMSVRLPPITKFAAEEARESDWDGIIACHQGKLSCSTWNYQKSTIGAYFLKPKELKKDDITATAVDITSCGNFAVIGLSSGTVDVYNMQSGIHRGSFGKDQAHKGSVRGVAVDGLNQLTVTTGSEGLLKFWNFKNKILIHSVSLSSSPNIMLLHRDSGILGLALDDFSISVLDIETRKIVREFSGHQGQINDMAFSPDGRWLISAAMDCSIRTWDLPSGCLIDCFLLDSAPLNVSMSPTGDFLATSHVDHLGIYLWSNISLYSVVSLRPLPADYVPSIVMLPGTCQTQDVEVSEETVEPSDELIEYDSPEQLNEQLVTLSLLPESRWKNLLNLDVIKKKNKPKEPPKVPKSAPFFIPTIPGLVPRYAAPEQNNDPQQSKVVNLGVLAQKSDFCLKLEEGLVNNKYDTALNLLKESGPSGIETELRSLSPDCGGSIEVMQSFLKMIGMMLDRKRDFELAQAYLALFLKLHLKMLPSEPVLLEEITNLSSQVEENWTHLQSLFNQSMCILNYLKSALL

SEQ ID NO: 9 - protein, 516 aa, Homo sapiens
MEREPGAAGVRRALGRRLEAVLASRSEANAVFDILAVLQSEDQEEIQEAVRTCSRLFGALLERGELFVGQLPSEEMVMTGSQGATRKYKVWMRHRYHSCCNRLGELLGHPSFQVKELALSALLKFVQLEGAHPLEKSKWEGNYLFPRELFKLVVGGLLSPEEDQSLLLSQFREYLDYDDTRYHTMQAAVDAVARVTGQHPEVPPAFWNNAFTLLSAVSLPRREPTVSSFYVKRAELWDTWKVAHLKEHRRVFQAMWLSFLKHKLPLSLYKKVLLIVHDAILPQLAQPTLMIDFLTRACDLGGALSLLALNGLFILIHKHNLEYPDFYRKLYGLLDPSVFHVKYRARFFHLADLFLSSSHLPAYLVAAFAKRLARLALTAPPEALLMVLPFICNLLRRHPACRVLVHRPHGPELDADPYDPGEEDPAQSRALESSLWELQALQRHYHPEVSKAASVINQALSMPEVSIAPLLELTAYEIFERDLKKKGPEPVPLEFIPAQGLLGRPGELCAQHFTLS

SEQ ID NO: 10 - protein, 2493 aa, Saccharomyces cerevisiae
MAKQRQTTKSSKRYRYSSFKARIDDLKIEPARNLEKRVHDYVESSHFLASFDQWKEINLSAKFTEFAAEIEHDVQTLPQILYHDKKIFNSLVSFINFHDEFSLQPLLDLLAQFCHDLGPDFLKFYEEAIKTLINLLDAAIEFESSNVFEWGFNCLAYIFKYLSKFLVKKLVLTCDLLIPLLSHSKEYLSRFSAEALSFLVRKCPVSNLREFVRSVFEKLEGDDEQTNLYEGLLILFTESMTSTQETLHSKAKAIMSVLLHEALTKSSPERSVSLLSDIWMNISKYASIESLLPVYEVMYQDFNDSLDATNIDRILKVLTTIVFSESGRKIPDWNKITILIERIMSQSENCASLSQDKVAFLFALFIRNSDVKTLTLFHQKLFNYALTNISDCFLEFFQFALRLSYERVFSFNGLKFLQLFLKKNWQSQGKKIALFFLEVDDKPELQKVREVNFPEEFILSIRDFFVTAEINDSNDLFEIYWRAIIFKYSKLQNTEIIIPLLERIFSTFASPDNFTKDMVGTLLKIYRKEDDASGNNLLKTILDNYENYKESLNFLRGWNKLVSNLHPSESLKGLMSHYPSLLLSLTDNFMLPDGKIRYETLELMKTLMILQGMQVPDLLSSCMVIEEIPLTLQNARDLTIRIKNVGAEFGKTKTDKLVSSFFLKYLFGLLTVRFSPVWTGVFDTLPNVYTKDEALVWKLVLSFIKLPDENQNLDYYQPLLEDGANKVLWDSSVVRLRDTIDTFSHIWSKYSTQNTSIISTTIERRGNTTYPILIRNQALKVMLSIPQVAENHFVDIAPFVYNDFKTYKDEEDMENERVITGSWTEVDRNVFLKTLSKFKNIKNVYSATELHDHLMVLLGSRNTDVQKLALDALLAYKNPTLNKYRDNLKNLLDDTLFKDEITTFLTENGSQSIKAEDEKVVMPYVLRIFFGRAQVPPTSGQKRSRKIAVISVLPNFKKPYINDFLSLASERLDYNYFFGNSHQINSSKATLKTIRRMTGFVNIVNSTLSVLRTNFPLHTNSVLQPLIYSIAMAYYVLDTESTEEVHLRKMASNLRQQGLKCLSSVFEFVGNTFDWSTSMEDIYAVVVKPRISHFSDENLQQPSSLLRLFLYWAHNPSLYQFLYYDEFATATALMDTISNQHVKEAVIGPIIEAADSIIRNPVNDDHYVDLVTLICTSCLKILPSLYVKLSDSNSISTFLNLLVSITEMGFIQDDHVRSRLISSLISILKGKLKKLQENDTQKILKILKLIVFNYNCSWSDIEELYTTISSLFKTFDERNLRVSLTELFIELGRKVPELESISKLVADLNSYSSSRMHEYDFPRILSTFKGLIEDGYKSYSELEWLPLLFTFLHFINNKEELALRTNASHAIMKFIDFINEKPNLNEASKSISMLKDILLPNIRIGLRDSLEEVQSEYVSVLSYMVKNTKYFTDFEDMAILLYNGDEEADFFTNVNHIQLHRRQRAIKRLGEHAHQLKDNSISHYLIPMIEHYVFSDDERYRNIGNETQIAIGGLAQHMSWNQYKALLRRYISMLKTKPNQMKQAVQLIVQLSVPLRETLRIVRDGAESKLTLSKFPSNLDEPSNFIKQELYPTLSKILGTRDDETIIERMPIAEALVNIVLGLTNDDITNFLPSILTNICQVLRSKSEELRDAVRVTLGKISIILGAEYLVFVIKELMATLKRGSQIHVLSYTVHYILKSMHGVLKHSDLDTSSSMIVKIIMENIFGFAGEEKDSENYHTKVKEIKSNKSYDAGEILASNISLTEFGTLLSPVKALLMVRINLRNQNKLSELLRRYLLGLNHNSDSESESILKFCHQLFQESEMSNSPQIPKKKVKDQVDEKEDFFLVNLESKSYTINSNSLLLNSTLQKFALDLLRNVITRHRSFLTVSHLEGFIPFLRDSLLSENEGVVISTLRILITLIRLDFSDESSEIFKNCARKVLNIIKVSPSTSSELCQMGLKFLSAFIRHTDSTLKDTALSYVLGRVLPDLNEPSRQGLAFNFLKALVSKHIMLPELYDIADTTREIMVTNHSKEIRDVSRSVYYQFLMEYDQSKGRLEKQFKFMVDNLQYPTESGRQSVMELINLIITKANPALLSKLSSSFFLALVNVSFNDDAPRCREMASVLISTMLPKLENKDLEIVEKYIAAWLKQVDNASFLNLGLRTYKVYLKSIGFEHTIELDELAIKRIRYILSDTSVGSEHQWDLVYSALNTFSSYMEATESVYKHGFKDIWDGIITCLLYPHSWVRQSAANLVHQLIANKDKLEISLTNLEIQTIATRILHQLGAPSIPENLANVSIKTLVNISILWKEQRTPFIMDVSKQTGEDLKYTTAIDYMVTRIGGIIRSDEHRMDSFMSKKACIQLLALLVQVLDEDEVIAEGEKILLPLYGYLETYYSRAVDEEQEELRTLSNECLKILEDKLQVSDFTKIYTAVKQTVLERRKERRSKRAILAVNAPQISADKKLRKHARSREKRKHEKDENGYYQRRNKRKRA

SEQ ID NO: 11 - protein, 923 aa, Saccharomyces cerevisiae
MKSDFKFSNLLGTVYRQGNITFSDDGKQLLSPVGNRVSVFDLINNKSFTFEYEHRKNIAAIDLNKQGTLLISIDEDGRAILVNFKARNVLHHFNFKEKCSAVKFSPDGRLFALASGRFLQIWKTPDVNKDRQFAPFVRHRVHAGHFQDITSLTWSQDSRFILTTSKDLSAKIWSVDSEEKNLAATTFNGHRDYVMGAFFSHDQEKIYTVSKDGAVFVWEFTKRPSDDDDNESEDDDKQEEVDISKYSWRITKKHFFYANQAKVKCVTFHPATRLLAVGFTSGEFRLYDLPDFTLIQQLSMGQNPVNTVSVNQTGEWLAFGSSKLGQLLVYEWQSESYILKQQGHFDSTNSLAYSPDGSRVVTASEDGKIKVWDITSGFCLATFEEHTSSVTAVQFAKRGQVMFSSSLDGTVRAWDLIRYRNFRTFTGTERIQFNCLAVDPSGEVVCAGSLDNFDIHVWSVQTGQLLDALSGHEGPVSCLSFSQENSVLASASWDKTIRIWSIFGRSQQVEPIEVYSDVLALSMRPDGKEVAVSTLKGQISIFNIEDAKQVGNIDCRKDIISGRFNQDRFTAKNSERSKFFTTIHYSFDGMAIVAGGNNNSICLYDVPNEVLLKRFIVSRNMALNGTLEFLNSKKMTEAGSLDLIDDAGENSDLEDRIDNSLPGSQRGGDLSTRKMRPEVRVTSVQFSPTANAFAAASTEGLLIYSTNDTILFDPFDLDVDVTPHSTVEALREKQFLNALVMAFRLNEEYLINKVYEAIPIKEIPLVASNIPAIYLPRILKFIGDFAIESQHIEFNLIWIKALLSASGGYINEHKYLFSTAMRSIQRFIVRVAKEVVNTTTDNKYTYRFLVSTDGSMEDGAADDDEVLLKDDADEDNEENEENDVVMESDDEEGWIGFNGKDNKLPLSNENDSSDEEENEKELP

SEQ ID NO: 12 - protein, 554 aa, Saccharomyces cerevisiae
MGHKKNGHRRQIKERENQNKFERSTYTNNAKNNHTQTKDKKLRAGLKKIDEQYKKAVSSAAATDYLLPESNGYLEPENELEKTFKVQQSEIKSSVDVSTANKALDLSLKEFGPYHIKYAKNGTHLLITGRKGHVASMDWRKGQLRAELFLNETCHSATYLQNEQYFAVAQKKYTFIYDHEGTELHRLKQHIEARHLDFLPYHYLLVTAGETGWLKYHDVSTGQLVSELRTKAGPTMAMAQNPWNAVMHLGHSNGTVSLWSPSMPEPLVKLLSARGPVNSIAIDRSGYYMATTGADRSMKIWDIRNFKQLHSVESLPTPGTNVSISDTGLLALSRGPHVTLWKDALKLSGDSKPCFGSMGGNPHRNTPYMSHLFAGNKVENLGFVPFEDLLGVGHQTGITNLIVPGAGEANYDALELNPFETKKQRQEQEVRTLLNKLPADTITLDPNSIGSVDKRSSTIRLNAKDLAQTTMDANNKAKTNSDIPDVKPDVKGKNSGLRSFLRKKTQNVIDERKLRVQKQLDKEKNIRKRNHQIKQGLISEDHKDVIEEALSRFG

SEQ ID NO: 13 - protein, 594 aa, Saccharomyces cerevisiae
MTMATTAMNVSVPPPDEEEQLLAKFVFGDTTDLQENLAKFNADFIFNEQEMDVEDQEDEGSESDNSEEDEAQNGELDHVNNDQLFFVDDGGNEDSQDKNEDTMDVDDEDDSSSDDYSEDSEEAAWIDSDDEKIKVPILVTNKTKKLRTSYNESKINGVHYINRLRSQFEKIYPRPKWVDDESDSELDDEEDDEEEGSNNVINGDINALTKILSTTYNYKDTLSNSKLLPPKKLDIVRLKDANASHPSHSAIQSLSFHPSKPLLLTGGYDKTLRIYHIDGKTNHLVTSLHLVGSPIQTCTFYTSLSNQNQQNIFTAGRRRYMHSWDLSLENLTHSQTAKIEKFSRLYGHESTQRSFENFKVAHLQNSQTNSVHGIVLLQGNNGWINILHSTSGLWLMGCKIEGVITDFCIDYQPISRGKFRTILIAVNAYGEVWEFDLNKNGHVIRRWKDQGGVGITKIQVGGGTTTTCPALQISKIKQNRWLAVGSESGFVNLYDRNNAMTSSTPTPVAALDQLTTTISNLQFSPDGQILCMASRAVKDALRLVHLPSCSVFSNWPTSGTPLGKVTSVAFSPSGGLLAVGNEQGKVRLWKLNHY

SEQ ID NO: 14 - protein, 593 aa, Saccharomyces cerevisiae
MSELFGVLKSNAGRIILKDPSATSKDVKAYIDSVINTCKNGSITKKAELDEITVDGLDANQVWWQVKLVLDSIDGDLIQGIQELKDVVTPSHNLSDGSTLNSSSGEESELEEAESVFKEKQMLSADVSEIEEQSNDSLSENDEEPSMDDEKTSAEAAREEFAEEKRISSGQDERHSSPDPYGINDKFFDLEKFNRDTLAAEDSNEASEGSEDEDIDYFQDMPSDDEEEEAIYYEDFFDKPTKEPVKKHSDVKDPKEDEELDEEEHDSAMDKVKLDLFADEEDEPNAEGVGEASDKNLSSFEKQQIEIRKQIEQLENEAVAEKKWSLKGEVKAKDRPEDALLTEELEFDRTAKPVPVITSEVTESLEDMIRRRIQDSNFDDLQRRTLLDITRKSQRPQFELSDVKSSKSLAEIYEDDYTRAEDESALSEELQKAHSEISELYANLVYKLDVLSSVHFVPKPASTSLEIRVETPTISMEDAQPLYMSNASSLAPQEIYNVGKAEKDGEIRLKNGVAMSKEELTREDKNRLRRALKRKRSKANLPNVNKRSKRNDVVDTLSKAKNITVINQKGEKKDVSGKTKKSRSGPDSTNIKL

SEQ ID NO: 15 - protein, 943 aa, Saccharomyces cerevisiae
MVKSYQRFEQAAAFGVIASNANCVWIPASSGNSNGSGPGQLITSALEDVNIWDIKTGDLVSKLSDGLPPGASDARGAKPAECTYLEAHKDTDLLAVGYADGVIKVWDLMSKTVLLNFNGHKAAITLLQFDGTGTRLISGSKDSNIIVWDLVGEVGLYKLRSHKDSITGFWCQGEDWLISTSKDGMIKLWDLKTHQCIETHIAHTGECWGLAVKDDLLITTGTDSQVKIWKLDIENDKMGGKLTEMGIFEKQSKQRGLKIEFITNSSDKTSFFYIQNADKTIETFRIRKEEEIARGLKKREKRLKEKGLTEEEIAKSIKESYSSFILHPFQTIRSLYKIKSASWTTVSSSKLELVLTTSSNTIEYYSIPYEKRDPTSPAPLKTHTIELQGQRTDVRSIDISDDNKLLATASNGSLKIWNIKTHKCIRTFECGYALTCKFLPGGLLVILGTRNGELQLFDLASSSLLDTIEDAHDAAIWSLDLTSDGKRLVTGSADKTVKFWDFKVENSLVPGTKNKFLPVLKLHHDTTLELTDDILCVRVSPDDRYLAISLLDNTVKVFFLDSMKFYLSLYGHKLPVLSIDISFDSKMIITSSADKNIKIWGLDFGDCHKSLFAHQDSIMNVKFLPQSHNFFSCSKDAVVKYWDGEKFECIQKLYAHQSEVWALAVATDGGFVVSSSHDHSIRIWEETEDQVFLEEEKEKELEEQYEDTLLTSLEEGNGDDAFKADASGEGVEDEASGVHKQTLESLKAGERLMEALDLGIAEIEGLEAYNRDMKLWQRKKLGEAPIKPQGNAVLIAVNKTPEQYIMDTLLRIRMSQLEDALMVMPFSYVLKFLKFIDTVMQNKTLLHSHLPLICKNLFFIIKFNHKELVSQKNEELKLQINRVKTELRSALKSTEDDLGFNVQGLKFVKQQWNLRHNYEFVDEYDQQEKESNSARKRVFGTVI

SEQ ID NO: 16 - protein, 817 aa, Saccharomyces cerevisiae
MDLKTSYKGISLNPIYAGSSAVATVSENGKILATPVLDEINIIDLTPGSRKILHKISNEDEQEITALKLTPDGQYLTYVSQAQLLKIFHLKTGKVVRSMKISSPSYILDADSTSTLLAVGGTDGSIIVVDIENGYITHSFKGHGGTISSLKFYGQLNSKIWLLASGDTNGMVKVWDLVKRKCLHTLQEHTSAVRGLDIIEVPDNDEPSLNLLSGGRDDIINLWDFNMKKKCKLLKTLPVNQQVESCGFLKDGDGKRIIYTAGGDAIFQLIDSESGSVLKRTNKPIEELFIIGVLPILSNSQMFLVLSDQTLQLINVEEDLKNDEDTIQVTSSIAGNHGIIADMRYVGPELNKLALATNSPSLRIIPVPDLSGPEASLPLDVEIYEGHEDLLNSLDATEDGLWIATASKDNTAIVWRYNENSCKFDIYAKYIGHSAAVTAVGLPNIVSKGYPEFLLTASNDLTIKKWIIPKPTASMDVQIIKVSEYTRHAHEKDINALSVSPNDSIFATASYDKTCKIWNLENGELEATLANHKRGLWDVSFCQYDKLLATSSGDKTVKIWSLDTFSVMKTLEGHTNAVQRCSFINKQKQLISCGADGLIKIWDCSSGECLKTLDGHNNRLWALSTMNDGDMIVSADADGVFQFWKDCTEQEIEEEQEKAKLQVEQEQSLQNYMSKGDWTNAFLLAMTLDHPMRLFNVLKRALGESRSRQDTEEGKIEVIFNEELDQAISILNDEQLILLMKRCRDWNTNAKTHTIAQRTIRCILMHHNIAKLSEIPGMVKIVDAIIPYTQRHFTRVDNLVEQSYILDYALVEMDKLF

SEQ ID NO: 17 - protein, 939 aa, Saccharomyces cerevisiae
MSIDLKKRKVEEDVRSRGKNSKIFSPFRIIGNVSNGVPFATGTLGSTFYIVTCVGKTFQIYDANTLHLLFVSEKETPSSIVALSAHFHYVYAAYENKVGIYKRGIEEHLLELETDANVEHLCIFGDYLCASTDDNSIFIYKKSDPQDKYPSEFYTKLTVTEIQGGEIVSLQHLATYLNKLTVVTKSNVLLFNVRTGKLVFTSNEFPDQITTAEPAPVLDIIALGTVTGEVIMFNMRKGKRIRTIKIPQSRISSLSFRTDGSSHLSVGTSSGDLIFYDLDRRSRIHVLKNIHRESYGGVTQATFLNGQPIIVTSGGDNSLKEYVFDPSLSQGSGDVVVQPPRYLRSRGGHSQPPSYIAFADSQSHFMLSASKDRSLWSFSLRKDAQSQEMSQRLHKKQDGGRVGGSTIKSKFPEIVALAIENARIGEWENIITAHKDEKFARTWDMRNKRVGRWTFDTTDDGFVKSVAMSQCGNFGFIGSSNGSITIYNMQSGILRKKYKLHKRAVTGISLDGMNRKMVSCGLDGIVGFYDFNKSTLLGKLKLDAPITAMVYHRSSDLFALALDDLSIVVIDAVTQRVVRQLWGHSNRITAFDFSPEGRWIVSASLDSTIRTWDLPTGGCIDGIIVDNVATNVKFSPNGDLLATTHVTGNGICIWTNRAQFKTVSTRTIDESEFARMALPSTSVRGNDSMLSGALESNGGEDLNDIDFNTYTSLEQIDKELLTLSIGPRSKMNTLLHLDVIRKRSKPKEAPKKSEKLPFFLQLSGEKVGDEASVREGIAHETPEEIHRRDQEAQKKLDAEEQMNKFKVTGRLGFESHFTKQLREGSQSKDYSSLLATLINFSPAAVDLEIRSLNSFEPFDEIVWFIDALTQGLKSNKNFELYETFMSLLFKAHGDVIHANNKNQDIASALQNWEDVHKKEDRLDDLVKFCMGVAAFVTTA

SEQ ID NO: 18 - protein, 552 aa, Saccharomyces cerevisiae
MVLLISEIKDIAKRLTAAGDRKQYNSIIKLINELVIPENVTQLEEDETEKNLRFLVMSLFQIFRKLFSRGDLTLPSSKKSTLEKEQFVNWCRKVYEAFKTKLLAIISDIPFETSLGLDSLDVYLQLAELESTHFASEKGAPFFPNKTFRKLIIALWSSNMGEIEDVKSSGASENLIIVEFTEKYYTKFADIQYYFQSEFNQLLEDPAYQDLLLKNVGKWLALVNHDKHCSSVDADLEIFVPNPPQAIENESKFKSNFEKNWLSLLNGQLSLQQYKSILLILHKRIIPHFHTPTKLMDFLTDSYNLQSSNKNAGVVPILALNGLFELMKRFNLEYPNFYMKLYQIINPDLMHVKYRARFFRLMDVFLSSTHLSAHLVASFIKKLARLTLESPPSAIVTVIPFIYNLIRKHPNCMIMLHNPAFISNPFQTPDQVANLKTLKENYVDPFDVHESDPELTHALDSSLWELASLMEHYHPNVATLAKIFAQPFKKLSYNMEDFLDWNYDSLLNAESSRKLKTLPTLEFEAFTNVFDNEDGDSEASSQGNVYLPGVAW

SEQ ID NO: 19 - protein, 440 aa, Saccharomyces cerevisiae
MSKTRYYLEQCIPEMDDLVEKGLFTKNEVSLIMKKRTDFEHRLNSRGSSINDYIKYINYESNVNKLRAKRCKRILQVKKTNSLSDWSIQQRIGFIYQRGTNKFPQDLKFWAMYLNYMKARGNQTSYKKIHNIYNQLLKLHPTNVDIWISCAKYEYEVHANFKSCRNIFQNGLRFNPDVPKLWYEYVKFELNFITKLINRRKVMGLINEREQELDMQNEQKNNQAPDEEKSHLQVPSTGDSMKDKLNELPEADISVLGNAETNPALRGDIALTIFDVCMKTLGKHYINKHKGYYAISDSKMNIELNKETLNYLFSESLRYIKLFDEFLDLERDYLINHVLQFWKNDMYDLSLRKDLPELYLKTVMIDITLNIRYMPVEKLDIDQLQLSVKKYFAYISKLDSASVKSLKNEYRSYLQDNYLKKMNAEDDPRYKILDLIISKL

(19) United States
(12) Patent Application Publication
(10) Pub. No.: US 2011/0183425 A1
(43) Pub. Date: Jul. 28, 2011

(54) Protein Complexes and Screening Methods

(75) Inventors: André Xavier de Carvalho Negräo Valente, Cantanhede (PT); Yuan Gao, Richmond, VA (US); Gregory A. Buck, Richmond, VA (US); Seth Roberts, Richmond, VA (US)

(73) Assignees: BIOCANT- ASSOCIACÃO DE TRANSFERÊNCIA DE TECHNOLOGIA, Cantanhede (PT); VIRGINIA COMMONWEALTH UNIVERSITY INTELLECTUAL PROPERTY FOUNDATION, Richmond, VA (US)

(21) Appl. No.: 12/997,375

(22) PCT Filed: Jun. 10, 2009
(86) PCT No.: PCT/EP09/04181

§ 371 Date: Dec. 10, 2010

Related U.S. Application Data
(60) Provisional application No. 61/131,933, filed on Jun. 13, 2008.

(30) Foreign Application Priority Data
Jun. 9, 2009 (PT) 104619

(51) Int. Cl. G01N 33/68; C07K 14/47

(52) U.S. Cl. 436/86; 530/409

(57) ABSTRACT

The application concerns an isolated protein complex comprising polypeptide components: (i) UTP20 HUMAN or a fragment, variant or homologue thereof; (ii) PWP2 HUMAN or a fragment, variant or homologue thereof; (iii) WDR46_HUMAN or a fragment, variant or homologue thereof; (iv) UTP18 HUMAN or a fragment, variant or homologue thereof; (v) MPPIO HUMAN or a fragment, variant or homologue thereof; (vi) WDR3_HUMAN or a fragment, variant or homologue thereof; (vii) TBL3 HUMAN or a fragment, variant or homologue thereof; (viii) WDR36_HUMAN or a fragment, variant or homologue thereof; and (ix) N0C4L HUMAN or a fragment, variant or homologue thereof. The application further concerns a method of identifying an agent that modulates the amount, function, activity, composition and/or formation of said protein complex; a method for the prevention or treatment of an eye disorder comprising administering to a subject in need thereof a suitable quantity of an agent that modulates the amount, function, activity, composition and/or formation of said protein complex; and a method of assessing whether a subject has or is likely to develop an eye disorder comprising determining whether the subject has an altered amount, function, activity, composition and/or formation of a protein complex.

Glaucoma is a group of diseases of the optic nerve involving loss of retinal ganglion cells in a characteristic pattern of optic neuropathy. Untreated glaucoma leads to permanent damage of the optic nerve and resultant visual field loss, which can progress to blindness. Once lost, this damaged visual field can never be recovered. Worldwide, it is the second leading cause of blindness: glaucoma affects one in two hundred people aged fifty and younger, and one in ten over the age of eighty, affecting over 67 million people worldwide.

Glaucoma can be categorised in to a number of different types: primary glaucoma and its variants including, primary open-angle glaucoma and primary closed-angle glaucoma; developmental glaucoma; secondary glaucoma; and absolute glaucoma.

People with a family history of glaucoma have about a six percent chance of developing glaucoma. Diabetics and those of African descent are three times more likely to develop primary open angle glaucoma. Asians are prone to develop angle-closure glaucoma, and Inuit have a twenty to forty times higher risk than caucasians of developing primary angle closure glaucoma. Women are three times more likely than men to develop acute angle-closure glaucoma due to their shallower anterior chambers. Use of steroids can also cause glaucoma.

Screening for glaucoma is usually performed as part of a standard eye examination performed by ophthalmologists and optometrists. Testing for glaucoma should include measurements of the intraocular pressure via tonometry, changes in size or shape of the eye, and an examination of the optic nerve to look for any visible damage to it, or change in the cup-to-disc ratio. If there is any suspicion of damage to the optic nerve, a formal visual field test should be performed. Scanning laser ophthalmoscopy may also be performed.

Owing to the sensitivity of some methods of tonometry to corneal thickness, methods such as Goldmann tonometry should be augmented with pachymetry to measure the cornea thickness. While a thicker-than-average cornea can cause a false-positive warning for glaucoma risk, a thinner-than-average cornea can produce a false-negative result. A false-positive result is safe, since the actual glaucoma condition will be diagnosed in follow-up tests. A false-negative is not safe, as it may suggest to the practitioner that the risk is low and no follow-up tests will be done.

Although intraocular pressure is only one major risk factors of glaucoma, lowering it via pharmaceuticals or surgery is currently the mainstay of glaucoma treatment. There are several different classes of medications to treat glaucoma with several different medications in each class. Commonly used medications include: Prostaglandin analogs like latanoprost (Xalatan), bimatoprost (Lumigan) and travoprost; topical beta-adrenergic receptor antagonists such as timolol, levobunolol (Betagan), and betaxolol; Alpha2-adrenergic agonists such as brimonidine; sympathomimetics like epinephrine and dipivefrin; Miotic agents (parasympathomimetics) like pilocarpine; Carbonic anhydrase inhibitors like dorzolamide (Trusopt), brinzolamide (Azopt), acetazolamide (Diamox). Each of these medicines may have local and systemic side effects. Adherence to medication protocol can be confusing and expensive; if side effects occur, the patient must be willing either to tolerate these, or to communicate with the treating physician to improve the drug regimen. Moreover, the possible neuroprotective effects of various topical and systemic medications are also being investigated.

In Europe, Japan, and Canada laser treatment is often the first line of therapy. In the U.S., adoption of early laser has lagged, even though prospective, multi-centered, peer-reviewed studies, since the early '90s, have shown laser to be at least as effective as topical medications in controlling intraocular pressure and preserving visual field.

There remains a need to identify both improved medicament for the treatment of glaucoma and methods of diagnosing this disorder.

Myopia is a refractive defect of the eye in which collimated light produces image focus in front of the retina when accommodation is relaxed. Those with myopia see nearby objects clearly but distant objects appear blurred. With myopia, the eyeball is too long, or the cornea is too steep, so images are focused in the vitreous inside the eye rather than on the retina at the back of the eye.

Various forms of myopia have been described by their clinical appearance: Simple myopia; Degenerative myopia; Nocturnal myopia; Pseudomyopia; Induced myopia. Myopia, which is measured in diopters by the strength or optical power of a corrective lens that focuses distant images on the retina, has also been classified by degree or severity. Low myopia usually describes myopia of −3.00 diopters or less. Medium myopia usually describes myopia between −3.00 and −6.00 diopters. High myopia usually describes myopia of −6.00 or more.

There are currently two basic mechanisms believed to cause myopia: form deprivation (also known as pattern deprivation) and optical defocus. Form deprivation occurs when the image quality on the retina is reduced; optical defocus occurs when light focuses in front of or behind the retina. Numerous experiments with animals have shown that myopia can be artificially generated by inducing either of these conditions. In animal models wearing negative spectacle lenses, axial myopia has been shown to occur as the eye elongates to compensate for optical defocus. The exact mechanism of this image-controlled elongation of the eye is still unknown.

Eyeglasses, contact lenses, and refractive surgery are the primary options to treat the visual symptoms of those with myopia. Hence there is also a need to identify both improved medicament for the treatment of myopia and methods of diagnosing this disorder.

Recently, it has been suggested that myopia is a risk factor for the development of glaucoma. Nearsighted patients have a twofold to threefold increased risk of glaucoma compared with those who are not nearsighted. This association is weak for eyes with low myopia but is significant for eyes with moderate-to-high myopia. Hence glaucoma and myopia may share some molecular disease pathways.

A first aspect of the invention provides an isolated protein complex comprising polypeptide components: (i) UTP20_HUMAN or a fragment, variant or homologue thereof; (ii) PWP2_HUMAN or a fragment, variant or homologue thereof; (iii) WDR46_HUMAN or a fragment, variant or homologue thereof; (iv) UTP18_HUMAN or a fragment, variant or homologue thereof; (v) MPP10_HUMAN or a fragment, variant or homologue thereof; (vi) WDR3_HUMAN or a fragment, variant or homologue thereof; (vii) TBL3_HUMAN or a fragment, variant or homologue thereof; (viii) WDR36_HUMAN or a fragment, variant or homologue thereof; and (ix) NOC4L_HUMAN or a fragment, variant or homologue thereof.

Systems Biology is the study of an organism, viewed as an integrated and interacting network of genes, proteins and biochemical reactions. The study of protein complexes and how they relate to disease states is one of the most prominent areas of Systems Biology. The Systems Biology approach is growing in importance in both academia and industry. Processes developed through Systems Biology strategies can be of high value to the pharmaceutical industry, particularly through the reduction of R&D time and costs through better drug target prediction and identification as well as optimizing clinical trial efficiency and strategy.

The inventors have utilised a method based on a computational algorithm that allows the prediction of protein complexes out of experimental data. This methodology maps cellular protein complexes and protein-protein interactions and can be utilised to identify protein complexes that may represent valuable therapeutic targets. These protein complex targets may then provide multiple opportunities to discover and develop new drugs for the treatment of disease. New information that associates protein complexes with human disease states may also allow the development of new diagnostics.

From this work the inventors have identified a protein complex from S. cerevisiae comprising the polypeptide components: YBA4_YEAST; PWP_YEAST; UTP7_YEAST; UTP18_YEAST; MPP10_YEAST; DIP2_YEAST; UTP13_YEAST; YL409_YEAST; NOC4_YEAST; and UTP6_YEAST.

It is important to point out that until the present application a protein complex containing such polypeptide components had not previously been recognised. Also, the protein complex of this aspect of the invention can have one or more further polypeptide components present, i.e. additional polypeptides to those listed above.

The inventors then identified human polypeptides homologous to the yeast polypeptide components of the protein complex set out above: UTP20_HUMAN is a homologue of YBA4_YEAST; PWP2_HUMAN is a homologue of PWP_YEAST; WDR46_HUMAN is a homologue of UTP7_YEAST; UTP18_HUMAN is a homologue of UTP18_YEAST; MPP10_HUMAN is a homologue of MPP10_YEAST; WDR3_HUMAN is a homologue of DIP2_YEAST; TBL3_HUMAN is a homologue of UTP13_YEAST; WDR36_HUMAN is a homologue of YL409_YEAST; and NOC4L_HUMAN is a homologue of NOC4_YEAST.

Following this work the inventors noted that the WDR36_HUMAN polypeptide has previously been linked to a form of adult-onset primary open angle glaucoma. This condition is associated with characteristic changes of the optic nerve head and visual field, often accompanied by elevated intraocular pressure. Furthermore the gene encoding UTP20_HUMAN polypeptide is located at 12q23.3, a chromosomal position identified as being linked to severe myopia. Severe myopia occurs primarily as a result of increased axial length of the eye, but it is known to be associated with glaucoma, cataracts and other ophthalmologic disorders. Both WDR36 and UTP30 are known to be expressed in the retina, and other tissues as well.

Additionally, as set out in Example 3 below the inventors further validated the association of genes coding for polypeptide components of the protein complex of the invention with congenital glaucoma. For this purpose patients and healthy individuals were genotyped by the inventors, searching for mutations in genes coding for the protein complex of the invention. From 18 high confident selected variants, 11 were further analyzed and 8 of these were statistically validated as associated with disease, in 5 genes encoding components of the protein complex. This data is of use in methods for assessing whether a subject has or is likely to develop an eye disorder (as discussed below).

Furthermore, it is important to point out that the presence of mutations in multiple genes demonstrates that the encoded polypeptides are associated in a common protein complex that is associated with congenital glaucoma.

The inventors have therefore concluded that a protein complex comprising the polypeptide components discussed above has a role in the development of eye disorders, particularly glaucoma and myopia.

It is important to point out that until the present application the role of a protein complex containing such polypeptide components in eye disorders, particularly glaucoma and myopia, had not previously been recognised.

The isolated protein complex of the first aspect of the invention can be of much use in, for example, the identification of agents for use in treating eye disorders, particularly glaucoma and myopia. Various “screening methods” using the protein complex are described further below.

By “eye disorders” we include a number of different ophthalmologic disorders, particularly glaucoma and myopia.

When used throughout this application, by “glaucoma” we include the different types of this disorder as discussed above, including: primary glaucoma and its variants including, primary open-angle glaucoma and primary closed-angle glaucoma; developmental glaucoma; secondary glaucoma; and absolute glaucoma.

By “myopia” we include the different types of this disorder as discussed above, including: Simple myopia; Degenerative myopia; Nocturnal myopia; Pseudomyopia; Induced myopia. We also include low myopia (usually describes myopia of −3.00 diopters or less; medium myopia (between −3.00 and −6.00 diopters); and high myopia (myopia of −6.00 or more).

By “isolated” we include where the protein complex is extracellular and is substantially pure of any contaminants; for example the isolated protein complex may be present in an aqueous solution in which it constitutes at least 50% of the total protein content of that solution; preferably 60%, 70%, 80%, 90%, 95%, or 99%. Methods of preparing an isolated protein complex according to the first aspect of the invention are discussed further below.

A “fragment” of said polypeptide will preferably comprise less than the total amino acid sequence of the full native polypeptide; preferably the fragment retains its biological activity.

A “variant” of the polypeptide also refers to a polypeptide wherein at one or more positions there have been amino acid insertions, deletions, or substitutions, either conservative or non-conservative, provided that such changes result in a protein whose basic properties, for example protein interaction, thermostability, activity in a certain pH-range (pH-stability) have not significantly been changed. “Significantly” in this context means that one skilled in the art would say that the properties of the variant may still be different but would not be unobvious over the ones of the original protein.

By “conservative substitutions” is intended combinations such as Gly, Ala; Val, Ile, Leu; Asp, Glu; Asn, Gln; Ser, Thr; Lys, Arg; and Phe, Tyr.

Such variants may be made using the methods of protein engineering and site-directed mutagenesis as would be well known to those skilled in the art.

By “fragment” or “variant” of the polypeptide component of the protein complex we include a polypeptide that can be present in the protein complex and is usable in the screening methods of the invention. Such a variant may be encoded by a gene in which different codons can be substituted which code for the same amino acid(s) as the original codons. Alternatively, the substitute codons may code for a different amino acid that will not affect the function or immunogenicity of the protein or which may improve its function or immunogenicity. For example, site-directed mutagenesis or other techniques can be employed to create single or multiple mutations, such as replacements, insertions, deletions, and transpositions.

We also include “fusions” of the polypeptide components in which said polypeptide is fused to any other polypeptide. For example, the said polypeptide may be fused to a polypeptide such as glutathione-S-transferase (GST) or protein A in order to facilitate purification of said polypeptide. Examples of such fusions are well known to those skilled in the art. Similarly, the said polypeptide may be fused to an oligo-histidine tag such as His6 or to an epitope recognised by an antibody such as the well known Myc tag epitope.

It will be recognised by those skilled in the art that the amino acid sequence of the polypeptide components of the protein complex of the invention can be used to identify homologues to that polypeptide (or nucleic acid encoding the polypeptide).

Methods by which homologues (or orthologues or paralogues) of polypeptides can be identified are well known to those skilled in the art: for example, in silico screening or database mining. Preferably, such polypeptides have at least 40% sequence identity, preferably at least 60%, at least 70%, at least 80%, at least 90% or at least 95% sequence identity to the polypeptide sequence of polypeptide components of the protein complex of the invention.

Methods of determining the percent sequence identity between two polypeptides are well known in the art. For example, the percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally. Further discussion concerning the calculation of percentage identities between different amino acid/polypeptide/nucleic acid sequences is presented later in the description.

Information concerning the amino acid sequence, and encoding nucleic acid sequence, of each of these polypeptides can be readily obtained from, for example, GenBank or UniProt, and can be easily obtained from those sources by a person skilled in the art. Examples of amino acid sequences of the polypeptide components of the protein complex of the invention are provided herein at the end of the description. Each of these examples also includes a URL for the UniProt entry (obtained by searching the database with the name of the polypeptide).

The polypeptide components, or fragments, variants or homologues thereof, may originate from any organism. However, a preferred embodiment of the first aspect of the invention is wherein the polypeptide components, or fragments, variants or homologues thereof, are mammalian; more preferably they are human.

Hence a preferred embodiment of this aspect of the invention is an isolated protein complex comprising polypeptide components: (i) UTP20_HUMAN or a fragment, variant or homologue thereof; (ii) PWP2_HUMAN or a fragment, variant or homologue thereof; (iii) WDR46_HUMAN or a fragment, variant or homologue thereof; (iv) UTP18_HUMAN or a fragment, variant or homologue thereof; (v) MPP10_HUMAN or a fragment, variant or homologue thereof; (vi) WDR3_HUMAN or a fragment, variant or homologue thereof; (vii) TBL3_HUMAN or a fragment, variant or homologue thereof; (viii) WDR36_HUMAN or a fragment, variant or homologue thereof; and (ix) NOC4L_HUMAN or a fragment, variant or homologue thereof.

In a preferred embodiment of the invention polypeptide components have the amino acid sequence provided in SEQ ID NOs:1 to 9. That is, the isolated protein complex comprises polypeptide components: UTP20_HUMAN (SEQ ID NO:1) or a fragment, variant or homologue thereof; (ii) PWP2_HUMAN (SEQ ID NO:2) or a fragment, variant or homologue thereof; (iii) WDR46_HUMAN (SEQ ID NO:3) or a fragment, variant or homologue thereof; (iv) UTP18_HUMAN (SEQ ID NO:4) or a fragment, variant or homologue thereof; (v) MPP10_HUMAN (SEQ ID NO:5) or a fragment, variant or homologue thereof; (vi) WDR3_HUMAN (SEQ ID NO:6) or a fragment, variant or homologue thereof; (vii) TBL3_HUMAN (SEQ ID NO:7) or a fragment, variant or homologue thereof; (viii) WDR36_HUMAN (SEQ ID NO:8) or a fragment, variant or homologue thereof; and (ix) NOC4L_HUMAN (SEQ ID NO:9) or a fragment, variant or homologue thereof.

Alternatively, a preferred embodiment of the first aspect of the invention is wherein the polypeptide components, or fragments, variants or homologues thereof; are yeast polypeptides; more preferably Saccharomyces cerevisiae.

As discussed above, the inventors identified a yeast protein complex having polypeptide components: YBA4_YEAST; PWP_YEAST; UTP7_YEAST; UTP18_YEAST; MPP10_YEAST; DIP2_YEAST; UTP13_YEAST; YL409_YEAST; NOC4_YEAST; and UTP6_YEAST.

Hence a preferred embodiment of this aspect of the invention is an isolated protein complex comprising polypeptide components: (i) YBA4_YEAST or a fragment, variant or homologue thereof; (ii) PWP_YEAST or a fragment, variant or homologue thereof; (iii) UTP7_YEAST or a fragment, variant or homologue thereof; (iv) UTP18_YEAST or a fragment, variant or homologue thereof; (v) MPP10_YEAST or a fragment, variant or homologue thereof; (vi) DIP2_YEAST or a fragment, variant or homologue thereof; (vii) UTP13_YEAST or a fragment, variant or homologue thereof; (viii) YL409_YEAST or a fragment, variant or homologue thereof; (ix) NOC4_YEAST or a fragment, variant or homologue thereof; and (x) UTP6_YEAST or a fragment, variant or homologue thereof.

In a preferred embodiment of the invention polypeptide components have the amino acid sequence provided in SEQ ID NOs:10 to 19. That is, the isolated protein complex comprises polypeptide components: (i) YBA4_YEAST (SEQ ID NO: 10) or a fragment, variant or homologue thereof; (ii) PWP_YEAST (SEQ ID NO:11) or a fragment, variant or homologue thereof; (iii) UTP7_YEAST (SEQ ID NO:12) or a fragment, variant or homologue thereof; (iv) UTP18_YEAST (SEQ ID NO:13) or a fragment, variant or homologue thereof; (v) MPP10_YEAST (SEQ ID NO:14) or a fragment, variant or homologue thereof; (vi) DIP2_YEAST (SEQ ID NO:15) or a fragment, variant or homologue thereof; (vii) UTP13_YEAST (SEQ ID NO:16) or a fragment, variant or homologue thereof; (viii) YL409_YEAST (SEQ ID NO:17) or a fragment, variant or homologue thereof; (ix) NOC4_YEAST (SEQ ID NO:18) or a fragment, variant or homologue thereof; and (x) UTP6_YEAST (SEQ ID NO:19) or a fragment, variant or homologue thereof.

A preferred embodiment of the first aspect of the invention is wherein at least one of the polypeptide components further comprise a fusion tag or label. Examples of such tags and labels are well known in the art, for example the HIS-tag and the GST tag, and may be of use in preparing an isolated protein complex of the invention.

As mentioned above, the isolated protein complex of the first aspect of the invention is extracellular and substantially pure of any contaminants.

The protein complex may be produced using a number of known techniques. For instance, the protein complex may be isolated from naturally occurring sources of the protein complex. Indeed, such naturally occurring sources of the protein complex may be induced to express increased levels of the protein complex, which may then be purified using well-known conventional techniques. Alternatively cells that do not naturally express the protein complex may be induced to express the polypeptide components of the protein complex.

It is possible to isolate the protein complex using a molecule which can specifically bind to at least one polypeptide component of the protein complex, such as an antibody. Using such a binding molecule in conditions that preserve the integrity of the protein complex, such an non-denaturing conditions, the polypeptide component, and hence the protein complex, can be isolated substantially pure of any contaminants.

For example, a culture of cells that contain the protein complex of the invention can be grown in vitro, the proteins extracted from the cells, and using an antibody to one of the polypeptide components of the protein complex, preferably under non-denaturing conditions, the polypeptide component, and hence the protein complex, can be isolated.

A further suitable technique to isolate the protein complex of the invention involves cellular expression of a fusion between a polypeptide component and a fusion tag or label, such as a his construct. The expressed polypeptide, and hence the protein complex, may subsequently be highly purified by virtue of the his “tag”.

Cells may be induced to express increased levels of the protein complex. This effect may be achieved either by manipulating the expression of endogenous polypeptide components of the protein complex, or causing the cultured cells to express exogenous polypeptide components of the protein complex. Expression of exogenous polypeptide components of the protein complex may be induced by transformation of cells with well-known vectors into which cDNA encoding polypeptide components of the protein complex may be inserted. It may be preferred that exogenous polypeptide components of the protein complex is expressed transiently by the cultured cell (for instance such that expression occurs only during ex vivo culture and ceases on administration of the cells to the subject requiring therapy).

As discussed above, information concerning nucleic acid sequences encoding polypeptide components of the isolated protein complex can be obtained from, for example, GenBank or UniProt, and can be easily obtained from those sources by a person skilled in the art.

It will be appreciated that the genes encoding the polypeptide components of the protein complex may be delivered to the biological cell without the gene being incorporated in a vector. For instance, the genes encoding the polypeptide components of the protein complex may be incorporated within a liposome or virus particle. Alternatively the “naked” DNA molecule may be inserted into the biological cell by a suitable means e.g. direct endocytotic uptake.

The exogenous genes encoding the polypeptide components of the protein complex (contained within a vector or otherwise) may be transferred to the biological cells by transfection, infection, microinjection, cell fusion, protoplast fusion or ballistic bombardment. For example, transfer may be by ballistic transfection with coated gold particles, liposomes containing the exogenous gene, and means of providing direct DNA uptake (e.g. endocytosis).

A second aspect of the invention provides a method of preparing an isolated protein complex according to the first aspect of the invention comprising:
(i) contacting a protein sample from a suitable target cell with one or more agent(s) that selectively bind one or more polypeptide components of the protein complex.
(ii) isolating the agent(s) and the attached protein complex from the protein sample.

Method of preparing an isolated protein complex according to the first aspect of the invention as discussed above in relation to that aspect of the invention.

Preferably the agent that selectively bind one or more polypeptide components of the protein complex is an antibody.

A further embodiment of this aspect of the invention is wherein one or more of the polypeptide components has a fusion tag or label to which the said agent(s) bind. Examples of such fusion tags or labels are discussed above and are well known in the art.

It is preferred that the method of this aspect of the invention is performed under “non-denaturing” conditions; as the skilled person would appreciate, by this term we include conditions that allow for maintenance of the integrity of the protein complex.

Preferably the isolated protein complex comprises polypeptide components, or fragments, variants or homologues thereof, that are mammalian; preferably human; and more preferably have the amino acid sequence provided in SEQ ID NOs:1 to 9.

A third aspect of the invention provides an isolated protein complex obtained or obtainable from the method of the second aspect of the invention. Preferably the protein complex is that obtained directly from the method of the second aspect of the invention.

The protein complex of the first aspect of the invention had not previously been identified. Moreover, as discussed above, the inventors have identified that the protein complex of the first aspect of the invention is likely to have a role in mediating disease, such as eye disorders, particularly glaucoma and myopia. This finding has lead to the inventors determining that the protein complex of the first aspect of the invention has much utility in the identification of agents that may be of use in the prevention or treatment of various eye disorders, most notably glaucoma. Such agents are those that can modulate a number of different aspects of the protein complex, such as the amount, function, activity, composition and/or formation, as discussed further below.

Therefore, a fourth aspect of the invention provides a method of identifying an agent that modulates the amount, function, activity, composition and/or formation of a protein complex according to the first aspect of the invention comprising:
(i) exposing the protein complex to a test agent; and,
(ii) determining the effect of the test agent on the amount, function, activity, composition and/or formation of the protein complex; and/or the amount, function and/or activity of one or more polypeptide components of the protein complex; and/or the amount of nucleic acid encoding one or more polypeptide components of the protein complex.

Preferably the method of the fourth aspect of the invention includes an additional step of selecting an agent that can modulate the amount, function, activity, composition and/or formation of the protein complex; and/or the amount, function and/or activity of one or more polypeptide components of the protein complex; and/or the amount of nucleic acid encoding one or more polypeptide components of the protein complex.

The protein complex used in the screening methods may be an isolated complex, i.e. extracellular, or the methods may use a cell having a protein complex of the invention, or an organism containing a protein complex of the invention.

By “protein complex” we include all embodiments of the protein complex discussed in relation to the first aspect of the invention.

The protein complex may be an isolated protein complex. That is, a sample of the isolated protein complex according to the first aspect of the invention can be prepared using the methods set out therein. In such circumstances the protein complex will be placed into a biologically suitable environment and then exposed to a quantity of the test agent. The effect of the test agent on the protein complex can then be determined using the experimental approaches set out below.

An embodiment of this aspect of the invention is wherein the protein complex is present within a suitable test cell. The cell could be any cell having the protein complex of the invention. However it is preferred that the cell is a mammalian cell containing a mammalian protein complex; preferably a human cell. Such a cell line could be a retinal pigment epithelial (RPE) cell line. Alternatively, it is preferred that the cell is a yeast cell; preferably Saccharomyces cerevisiae. We include cells including nucleic acid sequence encoding the specified polypeptide components of the protein complex of the invention. Such nucleic acid sequence may be a “native” gene present in the genome of that cell, or it may be a extrachromosomal nucleic acid molecule. Examples of nucleic acid sequence encoding the polypeptide components of the protein complex of the invention are discussed above.

A further embodiment of the fourth aspect of the invention is wherein the protein complex is present within an organism. The organism could be any organism having the protein complex of the invention. However it is preferred that the organism is a mammalian organism containing a mammalian protein complex; preferably not a human. Alternatively, it is preferred that the organism is a yeast; preferably Saccharomyces cerevisiae. We include organisms including nucleic acid sequence encoding the specified polypeptide components of the protein complex of the invention. Such nucleic acid sequence may be a “native” gene present in the genome of that organism, or it may be a extrachromosomal nucleic acid molecule. Examples of nucleic acid sequence encoding the polypeptide components of the protein complex of the invention are discussed above.

The methods of the fourth aspect of the invention are “screening methods” to identify agents of use in preventing or treating eye disorders, particularly glaucoma and myopia. For the reasons outlined above, an agent that modulates the amount, function, activity, composition and/or formation of a protein complex according to the first aspect of the invention is considered an agent that could be of use in preventing or treating eye disorders, particularly glaucoma and myopia.

In order to assess whether the test agent modulates the amount, function, activity, composition and/or formation of the protein complex, it is useful to compare the protein complex exposed to the test agent to a “reference sample”, i.e. a sample of the protein complex not exposed to the test agent. By comparing the protein complex in a sample exposed to the test agent, to a sample of protein complex not exposed to the test agent, it is possible to determine the effect of the test agent on the amount, function, activity, composition and/or formation of the protein complex; and/or the amount, function and/or activity of one or more polypeptide components of the protein complex; and/or the amount of nucleic acid encoding one or more polypeptide components of the protein complex.

Hence the test agent may produce an elevation, reduction or no effect on the amount of the protein complex or polypeptide components of the protein complex or the amount of nucleic acid encoding one or more polypeptide components of the protein complex; an alteration or no effect on the function of the protein complex or polypeptide components of the protein complex; a potentiation, inhibition or no effect on the activity of the protein complex or polypeptide components of the protein complex; an alteration or no effect on the composition of the protein complex; or an elevation, reduction or no effect on the formation of the protein complex.

The step of “determining the effect of the test agent on the amount, function, activity, composition and/or formation of the protein complex; and/or the amount, function and/or activity of one or more polypeptide components of the protein complex; and/or the amount of nucleic acid encoding one or more polypeptide components of the protein complex” may be performed using a number of different experimental techniques.

Non-exhaustive examples of methods of determining the amount of the protein complex or polypeptide components of the protein complex (and nucleic acids encoding such proteins) may be performed using a number of different methods, which are discussed below. Further information regarding some of the experimental procedures set out below are described further in Sambrook et al. (2000) Molecular Cloning, A Laboratory Manual, Cold Spring Harbor Laboratory, Cold Spring Harbor, N.Y.

Assaying protein levels in a sample can be performed using any art-known method. Total protein levels within a sample can be measured using Bradford reagent, fluorescamine dye or by using the Lowry method: these techniques are standard laboratory procedures.

It will be appreciated that the amount of a polypeptide may be measured by labelling a compound having affinity for that particular polypeptide. For example, antibodies, aptamers and the like may be labelled and used in an assay. Preferred for assaying protein levels in a biological sample are antibody-based techniques. Examples of immunoassays include immunofluorescence techniques known to the skilled technician, immunohistochemistry, enzyme-linked immunosorbent assay (ELISA), radioimmunoassay analyses.

Hence the amount of the protein complex can be determined using a compound having affinity for that particular polypeptide in the complex, then measuring the amount of the labelled protein complex using “non-denaturing” reaction conditions to maintain the interations between the components of the protein complex, as would be appreciated by the skilled person.

Also, the effect of a test agent on the amount of a polypeptide component of the protein complex can be measured using an antibody to that polypeptide, as part of techniques such as western blotting, immunohistochemistry and ELISA.

Levels of mRNA encoding particular polypeptides may be performed using the RT-PCR method. Briefly, this method involves converting mRNA isolated from a sample to cDNA using a reverse transcriptase enzyme. The cDNA products are then subject to PCR according to conventional techniques. After a suitable number of rounds to achieve amplification, the PCR reaction product corresponding to the mRNA encoding the particular polypeptide is quantified. Variations on the RT-PCR method will be apparent to the skilled artisan. Any set of oligonucleotide primers which will amplify reverse transcribed target mRNA can be used and can be designed as will be well known to those skilled in the art.

Levels of mRNA encoding the particular polypeptide can also be assayed using northern blotting, a method well known to those skilled in the art.

Further methods which may be of use in measuring mRNA levels include in situ hybridisation, in situ amplification, nuclease protection, probe arrays and amplification based systems. In addition, microarray analysis, a technique well known to those skilled in the art, may also be used to assess the amount of mRNA encoding a particular polypeptide.

Using such techniques common in the art, it would be possible to determine the amount of expression of particular polypeptide.

Also, the expression of a certain gene can be measured using promoter-reporter constructs, a technique well known to the skilled person.

The screening methods of the invention may also include assessing the effect of the test agent on the function of the protein complex or polypeptide components of the protein complex. In this respect, assays can be devised that examine the function of the protein complex, and polypeptide components of the complex, and the effect of the test agent on that function can be assessed; such as an alteration or no effect.

The screening methods of the invention may also include assessing the effect of the test agent on the activity of the protein complex or polypeptide components of the protein complex. In this respect, assays can be devised that examine the activity of the protein complex, and polypeptide components of the complex, and the effect of the test agent on that activity can be assessed; such as potentiation, inhibition or no effect.

In this respect, it is possible that the function/activity of the protein complex or polypeptide components of the protein complex has a role in RNA biogenesis and maturation, in which case appropriate conditions can be devised.

The screening methods of the invention may also include assessing the effect of the test agent on the composition of the protein complex. In this respect, assays can be devised that examine the composition of the protein complex, and the effect of the test agent on that composition can be assessed; such as an alteration or no effect. By “composition” we mean the different polypeptide components that make up the protein complex, and the relative quantities of those polypeptide components.

An example of an assay that can be used to assess the effect of the test agent on the composition of the protein complex would be to expose a suitable cell or organism containing the protein complex to a test agent, then isolate that protein complex, then examine the polypeptide composition of the complex and the relative amounts of the polypeptide components of the protein complex; such assays would use routine laboratory techniques.

The screening methods of the invention may also include assessing the effect of the test agent on the formation of the protein complex. In this respect, assays can be devised that examine the formation of the protein complex, and the effect of the test agent on that formation can be assessed; such as an elevation, reduction or no effect. By “formation” we include the stability of the complex, the rate at which the complex assembles and disassembles.

An example of an assay that can be used to assess the effect of the test agent on the formation of the protein complex would be to expose a suitable cell or organism containing the protein complex to a test agent, then isolate that protein complex, then examine the stability of the complex, for example over a period of time; such an assay would use routine laboratory techniques.

The screening methods of the invention relates to screening methods for drugs or lead compounds. The test agent may be a drug-like compound or lead compound for the development of a drug-like compound.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons and which may be water-soluble. A drug-like compound may additionally exhibit features of selective interaction with a particular protein or proteins and be bioavailable and/or able to penetrate target cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

The screening methods of the invention can be used in “library screening” methods, a term well known to those skilled in the art. Thus, for example, the methods of the invention may be used to detect (and optionally identify) a test agent that modulates the amount, function, activity, composition and/or formation of a protein complex according to the first aspect of the invention. Aliquots of a library may be tested for the ability to give the required result. Hence by “test compound”, we include where a protein complex is exposed to more than one compound at the same time, as is commonly performed in high throughput screening assays well known in the art.

An embodiment of the screening methods of the invention is wherein the method further comprises the step of selecting an agent that increases the amount, function, activity and/or formation of the protein complex.

By “increases” we include where the protein complex, or a cell or organism containing the protein complex, has, for example, 110%, 1250%, 130%, 140%, 150%, 200%, 250%, 500%, 1000%, or 10000% of the amount, function, activity and/or formation of the protein complex to that of the reference sample.

An embodiment of the screening methods of the invention is wherein the method further comprises the step of selecting a compound that decreases the amount, activity, composition and/or formation of the protein complex.

By “decreases” we include where the protein complex, or a cell or organism containing the protein complex, has, for example, 90%, 80%, 70%, 60%, 50%, 25%, 10%, 5%, 1%, or less. of the amount, activity, composition and/or formation of the protein complex to that of the reference sample. However, as the protein complex may participate in important biological processes, then it is preferred that any selected compound does not fully abolish the activity of the complex.

An embodiment of the screening methods of the invention is wherein the method further comprises the step of selecting an agent that alters the composition of the protein complex.

By “alters” we include where the protein complex, has at least one alteration to its is composition; this includes where one or more polypeptides are not present in the complex; where one or more polypeptide are additionally present in the complex; or where the relative amounts of polypeptide components of the complex is altered to that of the reference sample.

An embodiment of the screening methods of the invention is wherein the method has the additional step of mixing the selected agent (or a derivative or analogue thereof) with a pharmaceutically acceptable vehicle.

A fifth aspect of the invention provides a method of screening for compounds of use in preventing or treating an eye disorder, particularly glaucoma or myopia, wherein a non-human animal is administered a test agent and the effect of the test agent on the amount, activity, composition and/or formation of protein complex of the invention is assessed.

Preferably the eye disorder is glaucoma.

This aspect of the invention is also a “screening method”. The embodiments of the screening method aspects of the invention discussed above, and various techniques for performing the screening methods, also apply to this aspect of the invention.

The non-human animal may be any non-human animal, including non-human primates such as baboons, chimpanzees and gorillas, new and old world monkeys as well as other mammals such as cats, dogs, rodents, pigs or sheep, or other animals such as poultry, for example chickens, fish such as zebrafish, or amphibians such as frogs. However, it is preferred that the animal is a rodent such as a mouse, rat, hamster, guinea pig or squirrel. Preferably the animal is mouse. Preferably the non-human animal has a nucleic acid sequence encoding nucleic acid sequences encoding the polypeptide components of the protein complex of the invention.

An embodiment of this aspect of the invention is wherein the method further comprises the step of selecting an agent that increases the amount, function, activity and/or formation of the protein complex.

An embodiment of this aspect of the invention is wherein the method further comprises the step of selecting an agent that decreases the amount, function, activity and/or formation of the protein complex.

A sixth aspect of the invention provide a method of making a pharmaceutical composition comprising the screening method as described in the fourth and fifth aspects of the invention and the additional step of mixing the selected agent (or a derivative or analogue thereof) with a pharmaceutically acceptable carrier. Examples of such pharmaceutically acceptable vehicles are discussed further below.

According to a seventh aspect of the present invention, there is provided the use of an agent that modulates the amount, function, activity, composition and/or formation of a protein complex of the invention for the prevention or treatment of an eye disorder, particularly glaucoma or myopia.

According to an eighth aspect of the present invention, there is provided the use of an agent that modulates the amount, function, activity, composition and/or formation of a protein complex of the invention in the manufacture of a medicament for the prevention or treatment of an eye disorder, particularly glaucoma or myopia.

According to a ninth aspect of the invention there is provided a method of preventing or treating an eye disorder, particularly glaucoma or myopia, comprising administering to a subject a therapeutically effective quantity of an agent that modulates the amount, function, activity, composition and/or formation of a protein complex of the invention.

Agents of use in the seventh, eighth and ninth aspects of the invention, which modulate the amount, function, activity, composition and/or formation of a protein complex of the invention, are useful for preventing or treating an eye disorder, particularly glaucoma or myopia. Preferably the eye disorder is glaucoma.

Examples of agents which may be used according to the invention include where the agent may bind to the polypeptide components of the protein complex, or to the protein complex, and increase or prevent protein complex functional activity, e.g. antibodies and fragments and derivatives thereof (e.g. domain antibodies or Fabs). Alternatively the agent may act as a competitive inhibitor to the protein complex by acting as, for example, an antagonist. Alternatively the agent may be an activator of the protein complex by acting as an agonist. Alternatively the agent may inhibit or activate enzymes or other molecules in the protein complex biological pathway. Alternatively the agent may bind to mRNA encoding polypeptide components of the protein complex in such a manner as to lead to an increase or reduction in that mRNA and hence a modulation in the amount of protein complex.

Alternatively the agent may bind to a nucleic sequence encoding polypeptide components of the protein complex in such a manner that it leads to an increase or reduction in the amount of transcribed mRNA encoding polypeptide components of the protein complex. For instance the agent may bind to coding or non-coding regions of the genes or to DNA 5′ or 3′ of the genes and thereby reduce or increase expression of protein.

The agent may have been identified from the screening methods of the invention as being of use in the prevention or treatment of an eye disorder, particularly glaucoma or myopia.

An embodiment of the seventh, eighth and ninth aspects of the invention is wherein the agent increases the amount, function, activity and/or formation of the protein complex.

A further embodiment of the seventh, eighth and ninth aspects of the invention is wherein the agent is a polypeptide component of the protein complex.

In such an embodiment, the polypeptide may be administered directly to the subject. Alternatively, or additionally, in another embodiment of the invention, this may consists of administering a nucleic acid sequence encoding polypeptide to the subject, for example, by gene therapy. Gene therapy consists of the insertion or the introduction of a gene or genes into a subject in need of treatment.

It will be appreciated that there is some sequence variability between the sequence of the genes encoding polypeptide components of protein complex of the invention between genuses and species. Hence, it is preferred that the sequence of the gene used in the therapeutic aspects of the invention is from the same genus as that of the subject being treated. For example, if the subject to be treated is mammalian, then the methods according to the invention will use mammalian gene. It is especially preferred that the gene used is from the same species as that of the subject being treated. For example, if the subject to be treated is human, then the method according to the invention will use the human gene, and hence human polypeptide, and so on.

Preferably, the gene used in the methods according to the invention is substantially homologous to the subject's native gene, or a functional fragment thereof. Preferably, the degree of homology between the sequence of the gene used in the method and the sequence of the subject's native gene is at least 60% sequence identity, preferably, at least 75% sequence identity, preferably at least 85% identity; at least 90% identity; at least 95% identity; at least 97% identity; and most preferably, at least 99% identity.

Calculation of percentage identities between different amino acid/polypeptide/nucleic acid sequences may be carried out as follows. A multiple alignment is first generated by the ClustalX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof.

Suitably polypeptide for provision as a therapeutic agent may be produced by known techniques. For instance, the protein may be purified from naturally occurring sources of the polypeptide. Indeed, such naturally occurring sources of polypeptide may be induced to express increased levels of the protein, which may then be purified using well-known conventional techniques. Alternatively cells that do not naturally express the polypeptide may be induced to express such proteins. One suitable technique involves cellular expression of a polypeptide/his construct. The expressed construct may subsequently be highly purified by virtue of the his “tag”. Polynucleotide sequences encoding the polypeptide components of the protein complex are discussed herein.

It will be appreciated that polypeptide components of the protein complex represent favourable agents to be administered by techniques involving cellular expression of polynucleotide sequence encoding such polypeptides. Such methods of cellular expression are particularly suitable for medical use in which the therapeutic effects of the polypeptide are required over a prolonged period of time.

The genes may further comprise elements capable of controlling and/or enhancing its expression in the cell being treated. For example, the gene may be contained within a suitable vector to form a recombinant vector and preferably adapted to produce polypeptide. The vector may for example be a plasmid, cosmid or phage. Such recombinant vectors are highly useful in the delivery systems of the invention for transforming cells with the nucleic acid molecule. Example of suitable vectors include pCMV6-XL5 (OriGene Technologies Inc), NTC retroviral vectors (Nature Technology Corporation), adeno-associated viral vectors (Avigen Technology).

For human gene therapy, vectors will be used to introduce genes coding for products with at least 50%, 60%, 70%, 80%, 90%, 95% or 99% identity with the protein sequences provided herein.

State of the art vectors containing DNA coding for polypeptide components of the protein complex of the invention may be introduced into the blood stream. Any state of the art advantages of gene therapy (for example, considerably improved viral vectors derived from adeno-associated viruses, retroviruses, particularly lentiviruses) may be used to introduce DNA sequences.

It is preferred that at least 2 administrations of 1-1000 million units/ml is given at certain intervals, depending on vectors used (the vectors will influence the stability of expression and persistence of the desired polypeptide in organisms, from only several weeks to permanent expression) and individual requirements of the organism to be treated.

Recombinant vectors may comprise other functional elements to improve the gene therapy. For instance, recombinant vectors can be designed such that they will autonomously replicate in the cell in which they are introduced. In this case, elements that induce nucleic acid replication may be required in the recombinant vector. The recombinant vector may comprise a promoter or regulator to control expression of the gene as required. Alternatively, the recombinant vector may be designed such that the vector and gene integrates into the genome of the cell. In this case nucleic acid sequences, which favour targeted integration (e.g. by homologous recombination) may be desirable. Recombinant vectors may also have DNA coding for genes that may be used as selectable markers in the cloning process.

The gene may (but not necessarily) be one, which becomes incorporated in the DNA of cells of the subject being treated.

The delivery system may provide the gene the subject without it being incorporated in a vector. For instance, the nucleic acid molecule may be incorporated within a liposome or virus particle. Alternatively, a “naked” nucleic acid molecule may be inserted into a subject's cells by a suitable means e.g. direct endocytotic uptake.

The nucleic acid molecule may be transferred to the cells of a subject to be treated by transfection, infection, microinjection, cell fusion, protoplast fusion or ballistic bombardment. For example, transfer may be by ballistic transfection with coated gold particles, liposomes containing the nucleic acid molecule, viral vectors (e.g. adenovirus) and means of providing direct nucleic acid uptake (e.g. endocytosis) by application of the gene directly.

Polypeptide components of the protein complex of the invention or nucleic acid molecules encoding such polypeptides may be combined in compositions having a number of different forms depending, in particular on the manner in which the composition is to be used. Thus, for example, the composition may be in the form of a powder, tablet, capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and preferably enables delivery of the polypeptide or nucleic acid to the target cell, tissue, or organ. Hence, it is preferred that polypeptide is delivered by means of a suitably protected carrier particle, for example, a micelle.

Compositions comprising polypeptide or nucleic acid for use in the invention may be used in a number of ways. For instance, systemic administration may be required in which case the compound may be contained within a composition that may, for example, be ingested orally in the form of a tablet, capsule or liquid. Alternatively, the composition may be administered by injection into the blood stream. Injections may be intravenous (bolus or infusion) or subcutaneous (bolus or infusion). The compounds may be administered by inhalation (e.g. intranasally).

Polypeptide components of the protein complex of the invention or nucleic acid molecules encoding such polypeptides may also be incorporated within a slow or delayed release device. Such devices may, for example, be inserted on or under the skin, and the compound may be released over weeks or even months. Such devices may be particularly advantageous when long term treatment with a polypeptide or nucleic acids of use in the invention is required and which would normally require frequent administration (e.g. at least daily injection).

It will be appreciated that the amount of a polypeptide or nucleic acid that is required is determined by its biological activity and bioavailability which in turn depends on the mode of administration, the physicochemical properties of the polypeptide or nucleic acid employed, and whether the polypeptide or nucleic acid is being used as a monotherapy or in a combined therapy. Also, the amount will be determined by the number and state of target cells to be treated. The frequency of administration will also be influenced by the above-mentioned factors and particularly the half-life of the polypeptide or nucleic acid within the subject being treated.

Optimal dosages to be administered may be determined by those skilled in the art, and will vary with the particular polypeptide or nucleic acid in use, the strength of the preparation, the mode of administration, and the advancement of the disease condition. Additional factors depending on the particular subject being treated will result in a need to adjust dosages, including subject age, weight, gender, diet, and time of administration.

Known procedures, such as those conventionally employed by the pharmaceutical industry (e.g. in vivo experimentation, clinical trials, etc.), may be used to establish specific formulations of polypeptide or nucleic acid of use in the invention and precise therapeutic regimes (such as daily doses of the polypeptide or nucleic acid and the frequency of administration).

Generally, a daily dose of between 0.01 μg/kg of body weight and 0.5 g/kg of body weight of polypeptide or nucleic acid of use in the invention may be used for the prevention and/or treatment of glaucoma, depending upon which specific polypeptide or nucleic acid is used. More preferably, the daily dose is between 0.01 mg/kg of body weight and 200 mg/kg of body weight, and most preferably, between approximately 1 mg/kg and 100 mg/kg.

Daily doses may be given as a single administration (e.g. a single daily injection). Alternatively, the polypeptide or nucleic acid used may require administration twice or more times during a day. As an example, polypeptide or nucleic acid according to the invention may be administered as two (or more depending upon the severity of the condition) daily doses of between 25 mg and 7000 mg (i.e. assuming a body weight of 70 kg). A patient receiving treatment may take a first dose upon waking and then a second dose in the evening (if on a two dose regime) or at 3 or 4 hourly intervals thereafter. Alternatively, a slow release device may be used to provide optimal doses to a patient without the need to administer repeated doses.

This invention provides a pharmaceutical composition comprising a therapeutically effective amount of a polypeptide or nucleic acid of use in the invention and optionally a pharmaceutically acceptable vehicle. In one embodiment, the amount of the polypeptide or nucleic acid is an amount from about 0.01 mg to about 800 mg. In another embodiment, the amount of the polypeptide or nucleic acid is an amount from about 0.01 mg to about 500 mg. In another embodiment, the amount of the polypeptide or nucleic acid is an amount from about 0.01 mg to about 250 mg. In another embodiment, the amount of the polypeptide or nucleic acid is an amount from about 0.1 mg to about 60 mg. In another embodiment, the amount of the polypeptide or nucleic acid is an amount from about 0.1 mg to about 20 mg.

This invention provides a process for making a pharmaceutical composition comprising combining a therapeutically effective amount of a polypeptide or nucleic acid of use in the invention and a pharmaceutically acceptable vehicle. A “therapeutically effective amount” is any amount of polypeptide or nucleic acid of use in the invention which, when administered to a subject provides prevention and/or treatment of an eye disorder, particularly glaucoma or myopia. A “subject” is a vertebrate, mammal, domestic animal or human being.

A “pharmaceutically acceptable vehicle” as referred to herein is any physiological vehicle known to those of ordinary skill in the art useful in formulating pharmaceutical compositions.

A further embodiment of the seventh, eighth and ninth aspects of the invention is where the agent decreases the amount, function, activity and/or formation of the protein complex of the invention.

Agent for use in the seventh, eighth and ninth aspects of the invention may bind to polypeptide components of protein complex or to a nucleic acid encoding such polypeptides. Examples of nucleic acid and polypeptide sequences for protein complex are shown above.

When the agents binds to polypeptide components of the protein complex, it is preferred that the agent binds to an epitope defined by the polypeptide that has been correctly folded into its native form. It will be appreciated, that there can be some sequence variability between species and also between genotypes. Accordingly other preferred epitopes will comprise equivalent regions from variants of the gene. Equivalent regions from further polypeptides can be identified using sequence similarity and identity tools, and database searching methods, outlined herein. It is most preferred that the agent binds to a conserved region of the polypeptide or a fragment thereof.

An embodiment of the seventh, eighth and ninth aspects of the invention is wherein the agent is an antibody or fragment thereof.

The use of antibodies as agents to modulate polypeptide activity is well known. Indeed, therapeutic agents based on antibodies are increasingly being used in medicine. It is therefore apparent that such agents have great utility as medicaments for the improving the prevention or treatment of an eye disorder, particularly glaucoma or myopia. Moreover, such antibodies can be used in the prognostic methods set out below in further aspects of the invention.

Antibodies, for use in treating human subjects, may be raised against polypeptide components of the protein complex per se or a number of peptides derived from the polypeptide, or peptides comprising amino acid sequences corresponding to those found in the polypeptide.

It is preferred that the antibodies are raised against antigenic structures from human polypeptide components of the protein complex, and peptide derivatives and fragments thereof.

Antibodies may be produced as polyclonal sera by injecting antigen into animals. Preferred polyclonal antibodies may be raised by inoculating an animal (e.g. a rabbit) with antigen (e.g. all or a fragment of the polypeptide components of the protein complex) using techniques known to the art.

Alternatively the antibody may be monoclonal. Conventional hybridoma techniques may be used to raise such antibodies. The antigen used to generate monoclonal antibodies for use in the present invention may be the same as would be used to generate polyclonal sera.

In their simplest form, antibodies or immunoglobulin proteins are Y-shaped molecules usually exemplified by the IgG class of antibodies. The molecule consists of four polypeptide chains two identical heavy (H) chains and two identical (L) chains of approximately 50 kD and 25 kD each respectively. Each light chain is bound to a heavy chain (H-L) by disulphide and non-covalent bonds. Two identical H-L chain combinations are linked to each other by similar non-covalent and disulphide bonds between the two H chains to form the basic four chain immunoglobulin structure (H-L)₂.

Light chain immunoglobulins are made up of one V-domain (V_{L}) and one constant domain (C_{L}) whereas heavy chains consist of one V-domain and, depending on H chain isotype, three or four C-domains (C_{H}1, C_{H}2, C_{H}3 and C_{H}4).

At the N-terminal region of each light or heavy chain is a variable (V) domain that varies greatly in sequence, and is responsible for specific binding to antigen. Antibody specificity for antigen is actually determined by amino acid sequences within the V-regions known as hypervariable loops or Complementarity Determining Regions (CDRs). Each H and L chain V regions possess 3 such CDRs, and it is the combination of all 6 that forms the antibody's antigen binding site. The remaining V-region amino acids which exhibit less variation and which support the hypervariable loops are called frameworks regions (FRs).

The regions beyond the variable domains (C-domains) are relatively constant in sequence. It will be appreciated that the characterising feature of antibodies according to the invention is the V_{H} and V_{L} domains. It will be further appreciated that the precise nature of the C_{H} and C_{L} domains is not, on the whole, critical to the invention. In fact preferred antibodies for use in the invention may have very different C_{H} and C_{L} domains. Furthermore, as discussed more fully below, preferred antibody functional derivatives may comprise the Variable domains without a C-domain (e.g. scFV antibodies).

Preferred antibodies considered to be agents of use in the seventh, eighth and ninth aspects of the invention may have the V_{L} (first domain) and V_{H} (second domain) domains. A derivative thereof may have 75% sequence identity, more preferably 90% sequence identity and most preferably has at least 95% sequence identity. It will be appreciated that most sequence variation may occur in the framework regions (FRs) whereas the sequence of the CDRs of the antibodies, and functional derivatives thereof, should be most conserved.

A number of preferred embodiments of the agent of the seventh, eighth and ninth aspects of the invention relate to molecules with both Variable and Constant domains. However it will be appreciated that antibody fragments (e.g. scFV antibodies or FAbs) are also encompassed by the invention that comprise essentially the Variable region of an antibody without any Constant region.

An scFV antibody fragment considered to be an agent of the seventh, eighth and ninth aspects of the invention may comprise the whole of the V_{H} and V_{L} domains of an antibody raised against IFN polypeptide. The V_{H} and V_{L} domains may be separated by a suitable linker peptide.

Antibodies, and particularly mAbs, generated in one species are known to have several serious drawbacks when used to treat a different species. For instance when murine antibodies are used in humans they tend to have a short circulating half-life in serum and may be recognised as foreign proteins by the immune system of a patient being treated. This may lead to the development of an unwanted human anti-mouse antibody (HAMA) response. This is particularly troublesome when frequent administration of an antibody is required as it can enhance its clearance, block its therapeutic effect, and induce hypersensitivity reactions. These factors limit the use of mouse monoclonal antibodies in human therapy and have prompted the development of antibody engineering technology to generate humanised antibodies.

Therefore, where the antibody capable of modulating the amount, activity, composition and/or formation of the protein complex is to be used as a therapeutic agent for preventing or treating an eye disorder, particularly glaucoma or myopia, in a human subject, then it is preferred that antibodies and fragments thereof of non-human source are humanised.

Humanisation may be achieved by splicing V region sequences (e.g. from a monoclonal antibody generated in a non-human hybridoma) with C region (and ideally FRs from V region) sequences from human antibodies. The resulting ‘engineered’ antibodies are less immunogenic in humans than the non-human antibodies from which they were derived and so are better suited for clinical use.

Humanised antibodies may be chimeric monoclonal antibodies, in which, using recombinant DNA technology, rodent immunoglobulin constant regions are replaced by the constant regions of human antibodies. The chimeric H chain and L chain genes may then be cloned into expression vectors containing suitable regulatory elements and induced into mammalian cells in order to produce fully glycosylated antibodies. By choosing an appropriate human H chain c region gene for this process, the biological activity of the antibody may be pre-determined. Such chimeric molecules may be used to treat or prevent glaucoma.

Further humanisation of antibodies may involve CDR-grafting or reshaping of antibodies. Such antibodies are produced by transplanting the heavy and light chain CDRs of a non-human antibody (which form the antibody's antigen binding site) into the corresponding framework regions of a human antibody.

Humanised antibody fragments represent preferred agents for use according to the invention. Human FAbs recognising an epitope on protein complex of the invention, or polypeptide components of said complex, may be identified through screening a phage library of variable chain human antibodies. Techniques known to the art (e.g as developed by Morphosys or Cambridge Antibody Technology) may be employed to generate Fabs that may be used as agents according to the invention. In brief a human combinatorial Fab antibody library may be generated by transferring the heavy and light chain variable regions from a single-chain Fv library into a Fab display vector. This library may yield 2.1×10¹⁰ different antibody fragments. The peptide may then be used as “bait” to identify antibody fragments from then library that have the desired binding properties.

Domain antibodies (dAbs) represent another preferred agent that may be used according to this embodiment of the invention. dAbs are the smallest functional binding unit of antibodies and correspond to the variable regions of either the heavy or light chains of human antibodies. Such dAbs may have a molecule weight of around 13 kDa (corresponding to about 1/10 (or less) the size of a full antibody).

Further preferred agents that may be used according to this embodiment of the invention include bispecific Fab-scFv (a “bibody”) and trispecific Fab-(scFv)(2) (a “tribody”). For bibodies or tribodies, a scFv molecule is fused to one or both of the VL-CL (L) and VH-CH₁ (Fd) chains, e.g., to produce a tribody two scFvs are fused to C-term of Fab while in a bibody one scFv is fused to C-term of Fab. The preparation of such molecules can be routinely performed by the skilled person from information available in the field.

According to another embodiment of the seventh, eighth and ninth aspects of the invention, peptides may be used to modulate the amount, activity, composition and/or formation of the protein complex of the invention. Such peptides represent other preferred agents for use according to the invention. These peptides may be isolated, for example, from libraries of peptides by identifying which members of the library are able to modulate the amount or activation of polypeptide components of the protein complex of the invention. Suitable libraries may be generated using phage display techniques.

Aptamers represent another preferred agent of the seventh, eighth and ninth aspects of the invention. Aptamers are nucleic acid molecules that assume a specific, sequence-dependent shape and bind to specific target ligands based on a lock-and-key fit between the aptamer and ligand. Typically, aptamers may comprise either single- or double-stranded DNA molecules (ssDNA or dsDNA) or single-stranded RNA molecules (ssRNA). Aptamers may be used to bind both nucleic acid and non-nucleic acid targets. Accordingly aptamers may be generated that recognise and so modulate the activity or amount of the protein complex of the invention. Suitable aptamers may be selected from random sequence pools, from which specific aptamers may be identified which bind to the selected target molecules with high affinity. Methods for the production and selection of aptamers having desired specificity are well known to those skilled in the art, and include the SELEX (systematic evolution of ligands by exponential enrichment) process. Briefly, large libraries of oligonucleotides are produced, allowing the isolation of large amounts of functional nucleic acids by an iterative process of in vitro selection and subsequent amplification through polymerase chain reaction.

Antisense molecules represent another preferred agent for use according to the seventh, eighth and ninth aspects of the invention. Antisense molecules are typically single-stranded nucleic acids, which can specifically bind to a complementary nucleic acid sequence produced by a gene and inactivate it, effectively turning that gene “off”. The molecule is termed “antisense” as it is complementary to the gene's mRNA, which is called the “sense” sequence, as appreciated by the skilled person. Antisense molecules are typically are 15 to 35 bases in length of DNA, RNA or a chemical analogue. Antisense nucleic acids have been used experimentally to bind to mRNA and prevent the expression of specific genes. This has lead to the development of “antisense therapies” as drugs for the treatment of cancer, diabetes and inflammatory diseases. Antisense drugs have recently been approved by the US FDA for human therapeutic use. Accordingly, by designing an antisense molecule to polynucleotide sequence encoding polypeptide it would be possible to reduce the expression of that polypeptide in a cell and thereby reduce protein complex activity.

Small interfering RNA (siRNA), sometimes known as short interfering RNA or silencing RNA, represent further preferred agents for use according to the seventh, eighth and ninth aspects of the invention. It will be apparent that siRNA molecules that can reduce polypeptide expression may have utility in the preparation of medicaments for the prevention or treatment of glaucoma. siRNA are a class of 20-25 nucleotide-long RNA molecules are involved in the RNA interference pathway (RNAi), by which the siRNA can lead to a reduction in expression of a specific gene, or specifically interfere with the translation of such mRNA thereby inhibiting expression of protein encoded by the mRNA. siRNAs have a well defined structure: a short (usually 21-nt) double-strand of RNA (dsRNA) with 2-nt 3′ overhangs on either end. Each strand has a 5′ phosphate group and a 3′ hydroxyl (—OH) group. In vivo this structure is the result of processing by Dicer, an enzyme that converts either long dsRNAs or hairpin RNAs into siRNAs. siRNAs can also be exogenously (artificially) introduced into cells by various transfection methods to bring about the specific knockdown of a gene of interest. Essentially any gene of which the sequence is known can thus be targeted based on sequence complementarity with an appropriately tailored siRNA. Given the ability to knockdown essentially any gene of interest, RNAi via siRNAs has generated a great deal of interest in both basic and applied biology. There is an increasing number of large-scale RNAi screens that are designed to identify the important genes in various biological pathways. As disease processes also depend on the activity of multiple genes, it is expected that in some situations turning off the activity of a gene with a siRNA could produce a therapeutic benefit. Hence their discovery has led to a surge in interest in harnessing RNAi for biomedical research and drug development. Recent phase I results of therapeutic RNAi trials demonstrate that siRNAs are well tolerated and have suitable pharmacokinetic properties. siRNAs and related RNAi induction methods therefore stand to become an important new class of drugs in the foreseeable future. siRNA molecules designed to nucleic acid encoding polypeptide components of the protein complex of the invention can be used to reduce the expression of those polypeptides. Hence an embodiment of the seventh, eighth and ninth aspects of the invention is wherein the agent is a siRNA molecule having complementary sequence to polynucleotide encoding a component of the protein complex. Such polynucleotide sequences are discussed above.

Using such information it is straightforward and well within the capability of the skilled person to design siRNA molecules having complementary sequence to such polynucleotides. For example, a simple internet search yields many websites that can be used to design siRNA molecules.

By “siRNA molecule” we include a double stranded 20 to 25 nucleotide-long RNA molecule, as well as each of the two single RNA strands that make up a siRNA molecule.

It is most preferred that the siRNA is used in the form of hair pin RNA (shRNA). Such shRNA may comprise two complementary siRNA molecules that are linked by a spacer sequence (e.g. of about 9 nucleotides). The complementary siRNA molecules may fold such that they bind together.

A ribozyme capable of cleaving RNA or DNA encoding polypeptide components of the protein complex of the invention represent another preferred agent of the seventh, eighth and ninth aspect of the invention.

It will be appreciated that the amount of an agent needed according to the invention is determined by biological activity and bioavailability which in turn depends on the mode of administration and the physicochemical properties of the agent. The frequency of administration will also be influenced by the abovementioned factors and particularly the half-life of the agent within the target tissue or subject being treated.

Known procedures, such as those conventionally employed by the pharmaceutical industry (e.g. in vivo experimentation, clinical trials etc), may be used to establish specific formulations of the agents and precise therapeutic regimes (such as daily doses and the frequency of administration).

Generally, a daily dose of between 0.01 g/kg of body weight and 0.1 g/kg of body weight of an agent may be used in a treatment regimen for treating HCV infection; more preferably the daily dose is between 0.01 mg/kg of body weight and 100 mg/kg of body weight.

By way of example a suitable dose of an antibody according to the invention is 10 g/kg of body weight; 1 g/kg of body weight; 100 mg/kg of body weight, more preferably about 10 mg/kg of body weight; and most preferably about 6 mg/kg of body weight.

Daily doses may be given as a single administration (e.g. a single daily injection or a single dose from an inhaler). Alternatively the agent (e.g. an antibody or aptamer) may require administration twice or more times during a day.

Medicaments according to the invention should comprise a therapeutically effective amount of the agent and a pharmaceutically acceptable vehicle.

A “therapeutically effective amount” is any amount of an agent according to the invention which, when administered to a subject leads to an improvement in eye disorders, particularly glaucoma or myopia.

A “subject” may be a vertebrate, mammal, domestic animal or human being. It is preferred that the subject to be treated is human. When this is the case the agents may be designed such that they are most suited for human therapy (e.g. humanisation of antibodies as discussed above). However it will also be appreciated that the agents may also be used to treat other animals of veterinary interest (e.g. horses, dogs or cats).

A “pharmaceutically acceptable vehicle” as referred to herein is any physiological vehicle known to those skilled in the art as useful in formulating pharmaceutical compositions.

In one embodiment, the medicament may comprise between about 0.01 μg and 0.5 g of the agent. More preferably, the amount of the agent in the composition is between 0.01 mg and 200 mg, and more preferably, between approximately 0.1 mg and 100 mg, and even more preferably, between about 1 mg and 10 mg. Most preferably, the composition comprises between approximately 2 mg and 5 mg of the agent.

Preferably, the medicament comprises approximately 0.1% (w/w) to 90% (w/w) of the agent, and more preferably, 1% (w/w) to 10% (w/w). The rest of the composition may comprise the vehicle.

Nucleic acid agents can be delivered to a subject by incorporation within liposomes, Alternatively the “naked” DNA molecules may be inserted into a subject's cells by a suitable means e.g. direct endocytotic uptake. Nucleic acid molecules may be transferred to the cells of a subject to be treated by transfection, infection, microinjection, cell fusion, protoplast fusion or ballistic bombardment. For example, transfer may be by ballistic transfection with coated gold particles, liposomes containing the DNA molecules, viral vectors (e.g. adenovirus) and means of providing direct DNA uptake (e.g. endocytosis) by application of the DNA molecules directly to the target tissue topically or by injection.

The antibodies, or functional derivatives thereof, may be used in a number of ways. For instance, systemic administration may be required in which case the antibodies or derivatives thereof may be contained within a composition which may, for example, be ingested orally in the form of a tablet, capsule or liquid. It is preferred that the antibodies, or derivatives thereof, are administered by injection into the blood stream. Injections may be intravenous (bolus or infusion) or subcutaneous (bolus or infusion). Alternatively the antibodies may be injected directly to the liver.

Nucleic acid or polypeptide therapeutic entities may be combined in pharmaceutical compositions having a number of different forms depending, in particular on the manner in which the composition is to be used. Thus, for example, the composition may be in the form of a powder, tablet, capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and preferably enables delivery of the therapeutic to the target cell, tissue, or organ.

In a preferred embodiment, the pharmaceutical vehicle is a liquid and the pharmaceutical composition is in the form of a solution. In another embodiment, the pharmaceutical vehicle is a gel and the composition is in the form of a cream or the like.

Compositions comprising such therapeutic entities may be used in a number of ways. For instance, systemic administration may be required in which case the entities may be contained within a composition that may, for example, be ingested orally in the form of a tablet, capsule or liquid. Alternatively, the composition may be administered by injection into the blood stream. Injections may be intravenous (bolus or infusion) or subcutaneous (bolus or infusion). The entities may be administered by inhalation (e.g. intranasally).

Therapeutic entities may also be incorporated within a slow or delayed release device. Such devices may, for example, be inserted on or under the skin, and the compound may be released over weeks or even months. Such devices may be particularly advantageous when long term treatment with an entity is required and which would normally require frequent administration (e.g. at least daily injection).

A tenth aspect of the invention provides a method of assessing whether a subject has or is likely to develop an eye disorder, particularly glaucoma or myopia, comprising determining whether the subject has an altered amount, function, activity, composition and/or formation of a protein complex according to the invention.

The methods of the tenth aspect of the invention may be useful in the diagnosis of an eye disorder, particularly glaucoma or myopia, or as a basis of counseling if a subject is assessed as likely to develop such disorders.

As set out in Example 3 below the inventors further validated the association of genes coding for polypeptide components of the protein complex of the invention with congenital glaucoma. For this purpose patients and healthy individuals were genotyped by the inventors, searching for mutations in genes of the protein complex of the invention. From 18 high confident selected variants, 11 were further analyzed and 8 of these were statistically validated as associated with disease, in 5 genes encoding components of the protein complex.

Details of the 8 mutations statistically validated as associated with disease are provided in Table III in Example 3 below. They are: TBL3, nt 3895 G>A; UTP20, nt 10156 A>C; UTP20, nt 73119 T>C; WDR36, nt 191 T>C; WDR36, nt 6579 C>T; WDR36, nt 17980 G>A; PWP2, nt 14867 T>A; WDR3, nt 2019 T>G.

TBL3 nt 3895 G>A has SNP reference number rs35795901. PWP2 nt 14867 T>A has SNP reference number rs17856422. WDR3 nt 2019 T>G has SNP reference number rs41276602. Further information on these SNPs can be obtained from http://www.ncbi.nlm.nih.gov/projects/SNP/

Further information concerning variants UTP20, nt 10156 A>C; UTP20, nt 73119 T>C; WDR36, nt 191 T>C; WDR36, nt 6579 C>T; WDR36, nt 17980 G>A can be obtained from the relevant information for the gene from the NCBI database (http://www.ncbi.nlm.nih.gov/). The database entry for WDR36 is NG_{—}008979.1. The database entry for UTP20 is NC_{—}000012.10 (nucleotide region 100198036 to 100304528 selected). The nucleotide numbering used herein is that of the gene sequences available from the NCBI database for that gene.

On the basis of this information, it is possible to readily devise a method of this aspect of the invention, in which the presence of one or more of the specific mutations listed above in a subject is indicative that the subject has or is likely to develop an eye disorder. Further information is provided below as to how such methods can be performed.

Furthermore, 3 of the 8 mutations given above affect the amino acid sequence of the associated polypeptide: WDR36, nt 191 T>C causes a L25P change; WDR36, nt 6579 C>T causes a A163V change; and PWP2, nt 14867 T>A causes a F551I change. The amino acid numbering used in this paragraph is that shown in the sequence of the polypeptides given at the end of the specification.

Again, on the basis of this information, it is possible to readily devise a method of this aspect of the invention, in which the presence of one or more of the specific mutations listed above in a subject is indicative that the subject has or is likely to develop an eye disorder. Further information is provided below as to how such methods can be performed.

Preferably the eye disorder is glaucoma.

The method of the tenth aspect of the invention is performed using a sample of body fluid or tissue from the subject. The amount, function, activity, composition and/or formation of a protein complex of the invention in the subject is then compared that of the protein complex in a “control” sample or to known non-disease levels of the protein complex. The sample can be obtained from any tissue or body fluid that contains the protein complex. While it is preferred that the sample may be from the eye, since this may be difficult to obtain the sample can also be taken from a readily accessible source, such as while blood cells.

Methods of determining the amount, function, activity, composition and/or formation of a protein complex of the invention as mentioned above in relation to the screening methods of the invention and can be used in the tenth aspect of the invention.

Assaying protein levels in a biological sample can occur using any art-known method. Preferred for assaying protein levels in a biological sample are antibody-based techniques. For example, protein expression in tissues can be studied with classical immunohistological methods. In these, the specific recognition is provided by the primary antibody (polyclonal or monoclonal) but the secondary detection system can utilize fluorescent, enzyme, or other conjugated secondary antibodies. As a result, an immunohistological staining of tissue section for pathological examination is obtained. Tissues can also be extracted, e.g., with urea and neutral detergent, for the liberation of protein for Western-blot or dot/slot assay. In this technique, which is based on the use of cationic solid phases, quantitation of protein can be accomplished using isolated protein as a standard. This technique can also be applied to body fluids. With these samples, a molar concentration of protein will aid to set standard values of protein content for different body fluids, like serum, plasma, urine, spinal fluid, etc. The normal appearance of protein amounts can then be set using values from healthy individuals, which can be compared to those obtained from a test subject.

An embodiment of the tenth aspect of the invention is wherein if the sample has a altered amount, function, activity, composition and/or formation of a protein complex according to the invention then the subject is considered to be at risk of developing an eye disorder: for example an elevated amount, function, activity, and/or formation of the protein complex; a reduced amount, function, activity, and/or formation of the protein complex; an altered composition of the protein complex.

By “subject” we include a vertebrate, mammal, domestic animal or human; preferably the subject is human.

By “determining whether the subject has an altered amount, function, activity, composition and/or formation of a protein complex according to the invention”, we also include determining whether there are one or more mutations in the genes encoding the polypeptides components of the protein complex of the invention.

Information concerning the genes encoding the polypeptide components of the protein complex of the invention can be readily obtained from the information provided herein on the polypeptides; for example, by accessing the database entries for those proteins.

In the present invention, a mutation gene having a mutation is where the nucleic acid of the gene containing a mutation as compared to a wild type or normal gene nucleic acid. For example, a mutant gene can be a nucleic acid having the nucleotide sequence but including at least one mutation. By “mutation” as used herein with respect to nucleic acid, we include insertions of one or more nucleotides, deletions of one or more nucleotides, nucleotide substitutions, and combinations thereof, including mutations that occur in coding and non-coding regions (e.g., exons, introns, untranslated sequences, sequences upstream of the transcription start site of the coding mRNA, and sequences downstream of the transcription termination site of coding mRNA).

By “gene” we include the nucleic acid sequence that encodes the polypeptide or any fragment of that sequence. This can be genomic DNA sequence, mRNA sequence and cDNA sequence. Gene nucleic acid sequences include the untranslated regions extending both upstream of the transcription start site of coding mRNA and downstream of the transcription termination site of coding mRNA by, for example, 5 Kb. Coding gene nucleic acid sequences include all exon and intron sequences. We also include polymorphisms or variations in that nucleotide sequence that are naturally found between individuals of different ethnic backgrounds or from different geographical areas and which do not affect the function of the gene.

The method according to this aspect of the present invention is an in vitro method and can be performed on a sample containing nucleic acid derived from the subject. This requires isolation of genomic DNA from blood or saliva and subsequent sequence analysis of the genes encoding the proteins of the complex.

Various different approaches can be used to determine whether a subject has a mutation in a gene. These include determining the nucleic acid sequence of the gene; and determining the nucleic acid sequence of mRNA encoding the polypeptide. A further approach is to determine whether a subject has an alteration in the amino acid sequence of a polypeptide encoded by such a gene.

Information provided herein can be used to design materials, such as oligonucleotide primers or probes specific for each allele that can be used when determining the genotype of the gene of a subject. The design of such oligonucleotide primers is routine in the art and can be performed by the skilled person with reference to the information provided herein without any inventive contribution. If required, the primer(s) or probe(s) may be labelled to facilitate detection. Moreover, it is possible to determine the presence of alteration in the amino acid sequence of a polypeptide using binding agents, for example antibodies, which can distinguish for the presence of specific amino acids in a polypeptide, or by sequencing of polypeptides or fragments of polypeptides. Techniques that may be used to detect mutations include:—(1) Direct sequencing of the polymorphic region of interest (e.g. using commercially available kits such as the Cysts Thermo Sequence dye terminator kit-Amersham Pharmacia Biotech); (2) Sequence Specific Oligonucleotide Hybridization (SSO) (involving dot or slot blotting of amplified DNA molecules comprising the polymorphic region; hybridisation with labelled probes which are designed to be specific for each polymorphic variant; and detection of said labels); (3) Heteroduplex and single-stranded conformation polymorphism (SSCP) Analysis (involving analysis of electrophoresis band patterns of denatured amplified DNA molecules comprising the polymorphic region); (4) Sequence Specific Priming (SSP) [also described as Amplification Refractory Mutation System (ARMS)]; (5) Mutation Scanning [e.g. using the PASSPORT Mutation Scanning Kit (Amersham Pharmacia Biotech)]; (6) Chemical Cleavage of Mismatch Analysis; (7) Non-isotopic RNase Cleavage Assay (Ambion Ltd.); (8) Enzyme Mismatch Cleavage Assay; and (9) Single Nucleotide Extension Assay; (9) mass spectrometry analysis.

Furthermore, it is possible that genomic rearrangements can lead to mutations in the gene. Methods of determining genomic rearrangements include Southern blotting (essentially as performed as set out in Sambrook et al (1989). Molecular cloning, a laboratory manual, 2^{nd} edition, Cold Spring Harbor Press, Cold Spring Harbor, N.Y.) or quantitative PCR.

A further embodiment of this aspect of the invention is wherein the method comprises determining the nucleic acid sequence of mRNA encoding the polypeptide component.

Methods of isolating mRNA molecules from a sample are routine in the art and well known to the skilled person. Once isolated, the nucleotide sequence of the mRNA molecule can be determined, preferably from a cDNA sample prepared from mRNA isolated from the subject. The sequence of cDNA molecules can be determined according to the genotyping methods set out above.

The ability to be able to better determine the risk of and individual developing glaucoma or the progression of glaucoma very important for several reasons. Firstly, if an individual is incorrectly diagnosed as not having glaucoma when the individual does, in fact, have glaucoma, he or she may not be given appropriate treatment. Since it is particularly important that treatment is initiated at an early age in order to give the maximum chance of preventing progression of the disorder, a proper diagnosis is very desirable. Similarly if an individual is incorrectly diagnosed as having glaucoma when the individual does not, in fact, have glaucoma, he or she may be treated unnecessarily. Similarly, it is useful to determine whether a glaucoma subject is responding to a particular treatment. Similar considerations are relevant with respect to other eye disorders, such as myopia.

The inventors have determined that the protein complex of the invention is associated with eye disorders. As set out above, this finding is the basis for the methods of the tenth aspect of the invention in which the presence of an altered amount, function, activity, composition and/or formation of a protein complex according to the invention is indicative of a subject having or is likely to develop eye disorders, particularly glaucoma or myopia.

An eleventh aspect of the invention provides a non-human genetically modified animal having or predisposed to develop an eye disorder, particularly glaucoma or myopia, wherein the eye disorder results from an altered amount, function, activity, composition and/or formation of the protein complex of the invention.

Preferably the eye disorder is glaucoma.

Non-human animals with an altered amount, function, activity, composition and/or formation of the protein complex of the invention can be expected to develop an eye disorder and may therefore be useful in screening for potential therapeutic agents for preventing or treating such conditions.

The non-human animal may be any non-human animal, including non-human primates such as baboons, chimpanzees and gorillas, new and old world monkeys as well as other mammals such as cats, dogs, rodents, pigs or sheep, or other animals such as poultry, for example chickens, fish such as zebrafish, or amphibians such as frogs. However, it is preferred that the animal is a rodent such as a mouse, rat, hamster, guinea pig or squirrel. Preferably the animal is mouse.

By “altered amount, function, activity, composition and/or formation of the protein complex of the invention” we include that, in comparison to a normal animal of the same species or strain, the animal of the eleventh aspect of the invention has a reduced or elevated amount, function, activity, and/or formation of the protein complex; or an altered composition of the protein complex.

For example, the animal of this aspect of the invention may have the same amount of the protein complex, or polypeptide components of the complex per se, but the protein complex or polypeptide is in a non-functional state.

Alternatively, the altered amount of protein complex, or polypeptide components of the protein complex may be due to an altered amount of nucleic acid encoding the polypeptide components of the protein complex of the invention.

Methods of determining the amount, function, activity, composition and/or formation of the protein complex of the invention are provided herein in relation to other aspects of the invention. Preferably, “altered amount, function, activity, composition and/or formation of the protein complex of the invention” includes where the animal has an increased amount, for example, 110%, 1250%, 130%, 140%, 150%, 200%, 250%, 500%, 1000%, or 10000% of the amount, function, activity and/or formation of the protein complex; or a decreased amount, for example, 90%, 80%, 70%, 60%, 50%, 25%, 10%, 5%, 1% or less of the amount, activity, composition and/or formation of the protein complex; or an altered composition such that one or more polypeptides are not present in the complex; where one or more polypeptide are additionally present in the complex; or where the relative amounts of polypeptide components of the complex is altered to that of the reference sample.

The non-human animal of this aspect of the invention may have an altered amount, function, activity, composition and/or formation of the protein complex of the invention due to the animal being genetically modified so as to have an agent which can modify said protein complex function. For example the animal could be genetically modified to express a peptide or antibody which can bind to the protein complex and prevent function or sub-cellular localisation. The non-human animal of this aspect of the invention may have an altered amount of nucleic acid encoding polypeptide components of the protein complex according to the invention due to the animal being genetically modified so as to have an agent which can cause or induce degradation of said nucleic acid, for example a ribozyme which can target the nucleic acid, or an antisense molecule which can bind to the such nucleic acid. By “antisense” we include RNA interference (RNAi) technologies.

Alternatively, the animal may be genetically modified in such a manner as to alter the native gene(s) encoding polypeptide components of the protein complex according to the invention. Such an animal may be genetically modified for any of the genes encoding polypeptide components of the protein complex of the invention. However, it is preferred that that animal has alterations in genes encoding at least two different polypeptide components.

As mentioned above in relation to the tenth aspect of the invention, 8 mutations were statistically validated as associated with disease: TBL3, nt 3895 G>A; UTP20, nt 10156 A>C; UTP20, nt 73119 T>C; WDR36, nt 191 T>C; WDR36, nt 6579 C>T; WDR36, nt 17980 G>A; PWP2, nt 14867 T>A; WDR3, nt 2019 T>G. Hence one embodiment of this aspect of the invention is wherein the animal has one or more mutations, or mutations equivalent to those listed herein.

There are a number of different methods that can be employed to generate a non-human genetically modified animal according to this aspect of the invention. These will be discussed in turn below. Preferred methods include those in which the gene encoding the said polypeptide is altered or removed so as to produce little or none of said polypeptide. Other methods include inhibiting the transcription of the said gene or preventing any mRNA encoded by said gene from being translated due to the animal being genetically modified so as to have an agent which can modify said polypeptide transcription, translation and/or function.

Preferably, the methods set out below are employed to generate a non-human genetically modified animal according to this aspect of the invention in which the function of the protein complex is altered.

“Homologous recombination” is a technique well known to those skilled in the art. Animals in which an endogenous gene has been inactivated by homologous recombination are referred to as “knockout” animals. Hence this aspect of the invention includes wherein the amount, function, activity, composition and/or formation of the protein complex of the invention is altered by mutated one or more gene(s) encoding the polypeptide components by homologous recombination.

“Insertional mutagenesis” is also a term well known to those skilled in the art. Examples of such mutagenesis include transposon-tagging, homing endonuclease genes (HEGs). In such methods a region of DNA is introduced into a gene such that the controlling or coding region of the gene is disrupted. Such methods can be used to disrupt one or more genes encoding polypeptide components of the protein complex of the invention. As a result the animal will no longer be able to synthesise such polypeptide, i.e. there will be a reduction in the amount of this polypeptide and hence an alteration to the protein complex.

Chemical or physical mutagenesis can also be used in the method of this aspect of the invention. Here, a gene is mutated by exposing the genome to a chemical mutagen, for example ethyl methylsulphate (EMS) or ethyl Nitrosurea (ENU), or a physical mutagen, for example X-rays. Such agents can act to alter the nucleotide sequence of a gene or, in the case of some physical mutagens, can rearrange the order of sequences in a gene. Practical methods of using chemical or physical mutagenesis in animals are well known to those skilled in the art. Such methods can be used to disrupt one or more genes encoding polypeptide components of the protein complex. As a result the animal may no longer be able to synthesise such polypeptide, i.e. there will be a reduction in the amount and/or function of this polypeptide; alternatively the mutation may cause overactivity of the mutated polypeptide, i.e. there will be a increase in the amount and/or function of this polypeptide; alternatively, the mutation may cause an altered function of the mutated polypeptide.

Homologous recombination, insertional mutagenesis and chemical or physical mutagenesis can be used to generate a non-human animal which is heterozygous for a target gene. Such animals may be of particular use if the homozygous non-human animal has too severe a phenotype.

The non-human animal of this aspect of the invention could be genetically modified to include an antisense molecule or siRNA molecule that can affect the expression of polypeptide components of the protein complex.

Antisense oligonucleotides are single-stranded nucleic acids, which can specifically bind to a complementary nucleic acid sequence. By binding to the appropriate target sequence, an RNA-RNA, a DNA-DNA, or RNA-DNA duplex is formed. These nucleic acids are often termed “antisense” because they are complementary to the sense or coding strand of the gene. Recently, formation of a triple helix has proven possible where the oligonucleotide is bound to a DNA duplex. It was found that oligonucleotides could recognise sequences in the major groove of the DNA double helix. A triple helix was formed thereby. This suggests that it is possible to synthesise sequence-specific molecules which specifically bind double-stranded DNA via appropriate formation of major groove hydrogen bonds.

By binding to the target nucleic acid, the above oligonucleotides can inhibit the function of the target nucleic acid. This could, for example, be a result of blocking the transcription, processing, poly(A)addition, replication, translation, or promoting inhibitory mechanisms of the cells, such as promoting RNA degradations.

By “antisense” we also include all methods of RNA interference, which are regarded for the purposes of this invention as a type of antisense technology.

A further method of generating a non-human animal of this aspect of the invention is wherein the animal is genetically modified so as to have a ribozyme capable of cleaving RNA or DNA encoding polypeptide components of the protein complex.

A further method of generating a non-human animal of this aspect of the invention is wherein the animal is genetically modified so as to have an agent that acts as antagonist to polypeptide components of the protein complex.

The term “antagonist” is well known to those skilled in the art. By “antagonist” we include in this definition any agent that acts to alter the level and/or functional ability of polypeptide components of the protein complex. An example of an antagonist would include a chemical ligand that binds to and affects said polypeptide function, and in broader terms this could also include an antibody, or antibody fragment, that binds to one of the said polypeptides such that the polypeptide cannot effect its normal function. The antagonist may also alter the sub-cellular localisation of polypeptide. In this way, the amount of functional polypeptide is reduced.

A further method of generating a non-human animal of this aspect of the invention is wherein the animal is genetically modified so as to have a dominant inactive form of a polypeptide component of the protein complex.

The various elements required for a technician to perform the methods of aspects of the invention may be incorporated in to a kit.

A twelfth aspect of the invention provides a kit for assessing whether a subject has or is likely to develop an eye disorder, particularly glaucoma or myopia, comprising means for determining the amount, function, activity, composition and/or formation of a protein complex according to the invention.

Preferably the eye disorder is glaucoma.

By “means for determining the amount, function, activity, composition and/or formation of a protein complex according to the invention” we include the molecules given in the tenth aspect of the invention.

The kit of the twelfth aspect of the invention may also comprise relevant buffers and regents for conducting such methods.

The buffers and regents provided with the kit may be in liquid form and preferably provided as pre-measured aliquots. Alternatively, the buffers and regents may be in concentrated (or even powder form) for dilution.

All of the features described herein (including any accompanying claims, abstract and drawings), and/or all of the steps of any method or process so disclosed, may be combined with any of the above aspects in any combination, except combinations where at least some of such features and/or steps are mutually exclusive.

The invention will now be further described with reference to the following examples and Figures.

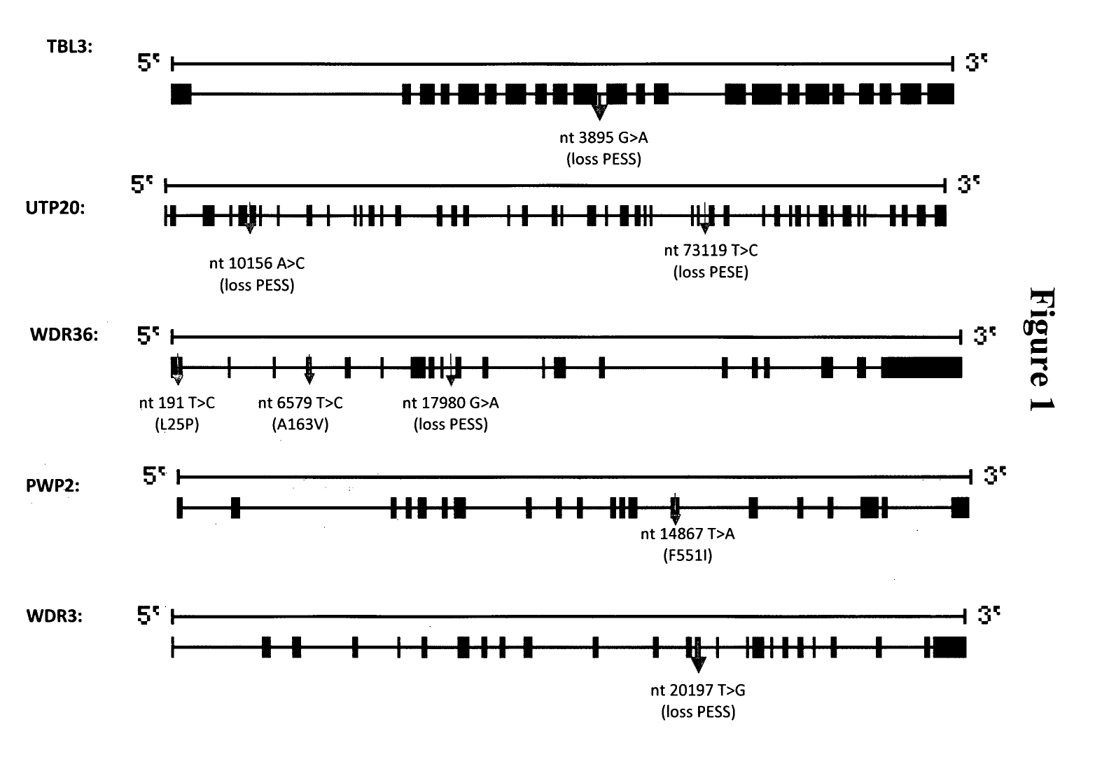

FIG. 1. Location of the sequence variants in the TBL3, UTP20, WDR36, PWP2 and WDR3 genes. Exons are represented as squares and introns as lines.

### EXAMPLE 1

### Identification of Protein Complexes Using an Algorithm Methodology

### Introduction

An important aim of proteomics is to identify which proteins interact; i.e. to identify a map of “protein-protein inteactions” within a given cell. The collection of protein physical interactions present in a cell, termed the “interactome”, constitutes a cornerstone in the field of “Systems Biology”, being the most fundamental level at which it is possible to perform an integrated analysis of a cell rather than just an isolated study of individual components.

Various experimental methods have been adopted to identify protein-protein interactions and protein complexes, such as for example affinity purification and yeast two hybrid (Y2H). Affinity purification is considered as a low-throughput method (LTP) suited to identify protein complexes. An advantage of this method is that there can be real determination of protein partners quantitatively in vivo without prior knowledge of complex composition. It is also simple to execute and often provides high yield. Y2H, in contrast, is suited to explore the binary interactions in mass quantities and is considered as high-throughput method (HTP). Each of the approaches has its own strengths and weaknesses, especially with regard to the sensitivity and specificity of the method. A high sensitivity means that many of the interactions that occur in reality are detected by the screen. A high specificity indicates that most of the interactions detected by the screen are also occurring in reality.

It is anticipated that the comprehensive mapping of protein physical interactions will facilitate the understanding of fundamental cell biology processes and the pathology of diseases. However, it is crucial to address two existing problems. Firstly, how obtain reliable interaction data in a high-throughput setting. This is important as high-throughput methods allow for the mapping of entire protein physical interactions present in a cell, i.e. an interactome. Secondly, how to structure interaction data in a meaningful form so as to be amenable and valuable for further biological research. This is important so as to identify protein interactions that constitute important protein complexes.

With this in mind, the inventors developed a method using a novel computational algorithm of analysing high-throughput interaction data to identify protein complexes. The inventors applied the method to construct a new interactome for S. cerevisiae, and demonstrated that it yields reliability typical of low-throughput experiments out of high-throughput data. Hence the method can be use to identify biologically important protein complexes, particularly those having a role in human disease.

### Results and Discussion

The inventors developed an algorithm to construct an interactome as proposed above, based on raw data from high-throughput affinity purification followed by mass spectrometric (AP-MS) identification assays (Gavin, A-C et al. (2002) Nature 415: 141-146; Gavin A-C et al. (2006) Nature 440 (7084):631-636; Krogan N J et al. (2006) Nature 440 (7084):637-643). The algorithm is suited for analyzing data from large-scale AP-MS interactome mapping projects, as the reliability (both sensitivity and specificity wise) of its predicted complexes improves as the number of AP-MS assays performed increases. Taking raw data from three large-scale AP-MS studies on S. cerevisiae (Gavin, A-C et al. (2002) Nature 415: 141-146; Gavin A-C et al. (2006) Nature 440 (7084):631-636; Krogan N J et al. (2006) Nature 440 (7084):637-643), they applied their methodology to build an S. cerevisiae interactome. The final interactome consists of 248 nodes (210 predicted multiprotein complexes and 38 single kinases) and 113 restricted transient interactions (65 predicted with their algorithm and 48 phosphorylation literature interactions).

The reliability of the data derived from the method of the invention was assessed using a number of different tests. Briefly, the protein complexes predicted according to the method of the invention were compared to manually curated complexes from the MIPS database; they were assessed using Semantic Distance analysis; and they were assessed according to an “essentiality” test. Taken together, the results from such analysis demonstrated that algorithm allows large-scale prediction of complexes with a reliability typical of low-throughput experiments from experimental data. Examples of protein complexes predicted from the method of this aspect of the invention are provided in the accompanying examples.

Certain pathologies may be assigned to an intrinsic malfunction of a complex as a whole, rather than to an individual or loose set of proteins (Kasper L et al. (2007) Nature Biotechnology 25: 309-316; Oti M, Snel M, Huynen M A, Brunner H G. (2006) Journal of Medical Genetics 43: 691-698; Chaudhuri A, Chant J. (2005) Bioessays 27: 958-969). With this in mind, the inventors extrapolated from the yeast interactome to human via homology (O'Brien K P, Remm M, Sonnhammer E L L. (2005) Nucleic Acids Research 33: D476-D480) and checked how known disease associated genes and chromosomal loci relate to their interactome map. Interestingly, a number of cases potentially pointing in this direction were found.

An example of related phenotypes mapping to the same complex is provided by a complex containing the gene PSMA6. A specific variant of this gene is known to confer susceptibility to myocardial infarction in the Japanese population (Ozaki K et al. (2006) Nat Genet. 38 (8): 921-5). A linkage to a related phenotype, susceptibility to premature myocardial infarction, has been reported at 1p36-34 (Wang Q. (2004) Am. J. Hum. Genet. 74 (2): 262-271) (no causative gene has yet been identified). This region includes PSMB2, another gene in the same complex. Linkage between various other cardiovascular phenotypes and genomic regions including genes from this complex have also been reported, e.g., linkage between familial atrial septal defect and 6p21.3 (Mohl W, Mayr W R. (1977) Tissue Antigens 10 (2): 121-2), a region that includes PSMB8 and PSMB9, genes that are also present in the complex. Hence the inventors conclude that the algorithm can identify biologically relevant protein complexes that can be linked with diseases.

A further example of a protein complex identified by the algorithm is the subject of the current patent application. The protein complex includes the S. cerevisiae polypeptide components: YBA4_YEAST; PWP_YEAST; UTP7_YEAST; UTP18_YEAST; MPP10_YEAST; DIP2_YEAST; UTP13_YEAST; YL409_YEAST; NOC4_YEAST; and UTP6_YEAST. The inventors then identified human polypeptides homologous to the yeast polypeptide components of the protein complex set out above: UTP20_HUMAN is a homologue of YBA4_YEAST; PWP2_HUMAN is a homologue of PWP_YEAST; WDR46_HUMAN is a homologue of UTP7_YEAST; UTP18_HUMAN is a homologue of UTP18_YEAST; MPP10_HUMAN is a homologue of MPP10_YEAST; WDR3_HUMAN is a homologue of DIP2_YEAST; TBL3_HUMAN is a homologue of UTP13_YEAST; WDR36_HUMAN is a homologue of YL409_YEAST; and NOC4L_HUMAN is a homologue of NOC4_YEAST.

Two possibly related phenotypes associated with polypeptide components of this protein complex. In this complex the gene, WDR36, was previously known to cause a form of adult-onset primary open angle glaucoma (Monemi S et al. Hum. Mol Genet. 14 (6): 725-33 (2005)). This condition is associated with characteristic changes of the optic nerve head and visual field, often accompanied by elevated intraocular pressure. Also in this complex is UTP20, located at 12q23.2. This gene falls within a chromosomal region identified as linked to severe myopia (Young TL et al. A Am J Hum Genet. 63 (5): 1419-24 (1998)) (the causative gene has not yet been identified). Severe myopia occurs primarily as a result of increased axial length of the eye, but it is known to be associated with glaucoma, cataracts and other ophthalmologic disorders (Curtin B J. The myopias: basic science and clinical management. Harpercollins College Div, Philadelphia (1985)). Both WDR36 and UTP20 are known to be expressed in the retina, and other tissues as well (Monemi S et al. Hum. Mol Genet. 14 (6): 725-33 (2005); Sharon D, Blackshaw S, Cepko C L, Dryja T P Proc. Natl. Acad. Sci. U.S.A. 99 (1): 315-20 (2002).

From the above it can be seen that the inventors have developed an algorithm that can be used to identify protein complexes from high-throughput interaction data. The algorithm can identify biologically relevant protein complexes that can be linked with diseases.

### EXAMPLE 2

### Experimental Methods for Isolating the Protein Complex of the Invention

Following the identification of the protein complex using the methodology set out in the example above, the inventors have identified a number of different and complementary experimental procedures to isolate the protein complex of the invention from different tissues or cells; preferably the cells are yeast cells. A discussion follows on a number of different procedures that can be adopted.

(A) Co-immunoprecipitation is a well established technique for protein interaction discovery. Co-immunopreciptation exploits the principles of immunoprecipitation (where an antibody against a specific target protein forms an immune complex which is then captured on a solid support to which either protein has been immobilized). In co-immunoprecipitation the target protein precipitated by the antibody “co-precipitates” a binding partner/protein complex from a lysate. Interacting proteins are subsequently identified by western blotting. Hence in the current case, an antibody the specifically binds to one of the protein components of the protein complex of the invention can be using to co-precipitate the other protein components of the complex. Also, if the experimental procedure is performed in “non-denaturing” conditions, then the procedure should isolate an intact protein complex.

(B) Fluorescence resonance energy transfer (FRET) is a common technique for observing interactions between two proteins. To monitor complex formation between two molecules, one molecule is labeled with a donor chromophore and the other with an acceptor chromophore—these fluorophore-labelled molecules are then mixed. When the molecules are dissociated, the emission by the donor chromophore is detected upon excitation of the donor. On the other hand, when the donor and acceptor are in proximity (1-10 nm) due to the interaction of the two molecules, the emission of the acceptor chromophore is predominantly observed because of the intermolecular FRET from the donor to the acceptor. Using this method, then, the interactions between the polypeptide components of the protein complex of the invention can be further studied.

(A) Pull-down assays are similar to immunoprecipitation methods but use a ligand other than an antibody to capture the protein complex. Pull-down methods are useful for both confirming the existence of a protein-protein interaction predicted by other research techniques and as an initial screening assay for identifying previously unknown interactions. The minimal requirement for a pull-down assay is the availability of a purified and tagged protein (the bait) which will be used to capture and pull-down the protein-binding partners (the prey). Pull-down assays exploit affinity purification methods similar to immunoprecipitation except that the bait protein is used instead of an antibody. Bait proteins can be generated either by linking an affinity tag to proteins purified by traditional purification methods or by expressing recombinant fusion-tagged proteins. Tandem Affinity Purification (TAP) involves the use of a tag to label the target protein of interest to create a TAP tag fusion which is then introduced into the host cell. The fusion protein present in extracts prepared from these cells, as well as the associated components, are then recovered by Tandem Affinity Purification (TAP). Hence in the current case, a ligand that specifically binds to one of the protein components of the protein complex of the invention can be used to co-precipitate the other protein components of the complex. Again, if the experimental procedure is performed in “non-denaturing” conditions, then the procedure should isolate an intact protein complex.

(D) Label transfer can be used for screening or confirmation of protein interactions and can provide information about the interface where the interaction takes place. Label transfer can also detect weak or transient interactions that are difficult to capture using other in vitro detection strategies. Label transfer involves cross-linking interacting molecules (i.e., bait and prey proteins) with a labeled cross-linking agent and then cleaving the linkage between bait and prey such that the label remains attached to the prey. This method enables the identification of proteins that interact weakly or transiently with a protein of interest. Hence the method can be used to further study interactions between the polypeptide components of the protein complex of the invention.

(E) The yeast two-hybrid screen investigates the interaction between artificial fusion proteins inside the nucleus of yeast. This approach can identify binding partners of a protein in an unbiased manner. However, it is necessary to verify the identified interactions by co-immunoprecipitation. The yeast 2 hybrid system is very useful for studying protein-protein interactions where it is speculated that 2 proteins interact. The Yeast 3 hybrid system also exists where a chaperone protein is necessary for the protein interaction to take place. Yeast 2 hybrid assays involve the subcloning of genes (relating to the proteins of interest) into vectors with a transcriptional activator of a fluorescent reporter gene (eg Beta-Gal or Lex A) into yeast. One vector contains the DNA binding domain while the other vector contains the activation domain. Two fusion proteins are then created 1) the protein of interest which has the DNA binding domain attached to its N-terminus—the bait and 2) its potential binding partner which has the activation domain—the prey If the proteins interact, the binding of these will result in the formation of a functional transcriptional activator, which will then go on to transcribe the reporter gene. The protein product of the reporter gene can then be easily detected and measured. Hence the method can be used to further study interactions between the polypeptide components of the protein complex of the invention.

(F) Chemical cross-linking is used to prevent the disassociation of complexes during analysis by methodologies such as mass spectrometry. Common crosslinking compounds include 1) Bis(Sulfosuccinimidyl)suberate (BS3), a water-soluble, non-cleavable and membrane impermeable crosslinker 2) 3,3′-Dithiobis(sulfosuccinimidylpropionate) (DTSSP), a water-soluble, thiol-cleavable and membrane impermeable crosslinker and 3) Dimethyl dithiobispropionimidate (DTBP), a cleavable and membrane permeable cross-linker. Another method of cross-linking involves the use of photo-reactive amino acid analogues which can be used in intact cells. Cells are grown with photoreactive diazirine analogues to leucine and methionine, incorporated into their proteins. Upon exposure to ultraviolet light, the diazirines are activated and bind to interacting proteins that are within a few angstroms of the photo-reactive amino acid analogue. Thus in the current case, the method can be used to identify and further study the protein complex.

### EXAMPLE 3

### Mutation Screening in Genes of the Protein Complex Associated with Congenital Glaucoma

### Objectives

The inventors sought to further validate the association of genes coding for the protein complex of the invention with congenital glaucoma. For this purpose patients and healthy individuals were genotyped searching for mutations in genes of the protein complex predicted to be associated with the disease.

### Methodology and Results

### Primary Congenital Glaucoma and Protein Complex Genes

Primary congenital glaucoma (PCG) is a severe form of glaucoma that tends to be diagnosed in the first few months of life but in some cases may not be diagnosed until much later in infancy (up to 3 years old). The disease occurs in 1 of 10,000 births in Western Countries and accounts for 2 to 15% cases among children in institutions for the blind. Primary congenital glaucoma is characterised by the improper development of the trabecular meshwork and in many cases appears to be an autosomal recessive inherited disorder. Mutations in the CYP1B1 gene (which encodes Cytochrome P450 1B1 and is expressed in the trabecular meshwork) have already been identified as being a cause of PCG with CYP1B1 mutations being found in 20-30% of patients with PCG.

Mutation screening in a set of PCG patients was conducted to investigate whether the genes (UTP20, MPP10, WDR46, NOC4L, WDR36, TBL3, UTP18, PWP2 and WDR3) encoding polypeptides involved in this protein complex are also linked with susceptibility to glaucoma, using healthy individuals as controls.

### Sequencing of Exons

A total of 222 fragments with an average length of 200 bp, corresponding to the exons and exon/intron boundaries of CYP1B1, UTP20, MPP10, WDR46, NOC4L, WDR36, TBL3, UTP18 and PWP2 genes, were sequenced in 17 PCG patients and in 17 controls using a massively parallel sequencing approach (Table I). This is a well suited technology for mutational analysis in large populations, which allows massive parallel picoliter-scale amplification and pyrosequencing of individual DNA molecules.

TABLE I
Number of sequenced exons and fragments in each gene.

| | Gene | | |
|---|---|---|---|
| Gene | length (kb) | Exons | Fragments |
| CYP1B1 | 8.55 | 2 | 8 |
| UTP20 | 106.49 | 62 | 65 |
| MPP10 | 19.79 | 11 | 14 |
| WDR46 | 10.11 | 15 | 15 |
| NOC4L | 7.99 | 15 | 13 |
| WDR36 | 38.33 | 23 | 25 |
| TBL3 | 6.69 | 22 | 21 |
| UTP18 | 37.4 | 14 | 14 |
| PWP2 | 23.86 | 21 | 21 |
| WDR3 | 30.66 | 27 | 26 |
| | | 212 | 222 |

Specific oligonucleotides, tagged with sequencing adaptors, were designed for amplification of these fragments using Primer3 and OligoExplorer softwares. Genomic DNA of patients and controls were isolated, accurately quantified by fluorimetry (PicoGreen dsDNA quantitation reagent) and mixed in two equimolar pools that were independently used as templates for amplification of the 222 fragments. The amplicons were purified with AMPure magnetic beads, visualized in an automated capillary electrophoresis system (Caliper Life Sciences) and quantified by use of PicoGreen. Clonal amplification on beads (emulsion PCR) was performed from equimolar pools of all amplicons per sample of patients and controls. After bead isolation, enriched DNA-containing beads were counted and loaded on a PicoTiter plate. Sequencing was performed on a Genome Sequencer FLX (Roche—454 Life Sciences).

### Analysis of the Sequence Reads

Nucleotide reads obtained in the massively parallel sequencing were aligned to the respective consensus sequence (NCBI databases) by Amplicon Variant Analyzer (AVA) software. Variant screening analysis of the 10 genes in patients and controls unveiled a total of 545 variants (Table in Annex I).

It is known that, using this sequencing approach, certain features and positions of the amplicons are particularly susceptible to error. The identification of these errors has permitted the establishment of criteria to select high confident variants. Such errors include: 1) errors are predominantly frequent around runs of 4, or more bases of the same nucleotide, known “homopolymer tracts”; 2) there are more errors of all types toward the beginning and the end of the sequence. In addition, only the variants exclusively detected in the patients set or in a significant greater proportion were considered as potentially involved in the disease.

From the 545 variants previously identified 18 high confident variants were selected (Table II). The variants were identified in exonic regions associated with amino acid changes and in intronic regions related with loss or gain of splicing regulatory motifs, such as ESS (exonic splicing silencer) and ESE (exonic splicing enhancer) elements (Table II). It was also analyzed whether these variants had been already described as SNP using NCBI database (http://www.ncbi.nlm.nih.gov/) (Table II).

TABLE II
Variants selected from those 545 obtained by massively parallel
sequencing of congenital glaucoma patients and controls.

| Gene | Variant | Region | Effect | SNP | Frequency (P) | | Frequency (C) |
|---|---|---|---|---|---|---|---|
| TBL3 | nt 3895 G > A | Intron 11 | loss of PESS | rs35795901 | 6.7% | (1 homoz) | 0 |
| MPP10 | nt 2701 A > C | Exon 2 | E69A | rs10199088 | 24.4% | (4 homoz) | 11.5% (2 homoz) |
| MPP10 | nt 3028 G > A | Exon 2 | S178N | | 20.5% | (3.5) | 13.9% (2 homoz) |
| MPP10 | nt 3150 A > G | Exon 2 | K219E | | 2.9% | (1 heteroz) | 0 |
| MPP10 | nt 19556 G > A | Exon 11 | E634K | | 31.8% | (5 homoz) | 16.5% (3 homoz) |
| UTP18 | nt 8216 A > G | Intron 3 | | | 3.3% | (1 heteroz) | 0 |
| UTP20 | nt 5754 G > A | Exon 4 | D109N | | 7.0% | (1 homoz) | 3.7% (1 heteroz) |
| UTP20 | nt 10156 A > C | Intron 7 | loss of PESS | | 3.0% | (1 heteroz) | 0 |
| UTP20 | nt 64502 A > G | Exon 36 | M1495V | | 8.8% | (1.5) | 0 |
| UTP20 | nt 73119 T > C | Intron 40 | loss of PESE | | 2.9% | (1 heteroz) | 0 |
| UTP20 | nt 81779 T > C | Intron 43 | loss of PESS | rs7313312 | 28.6% | (5 homoz) | 11.8% (2 homoz) |
| UTP20 | nt 89599 T > C | Exon 49 | I2130T | | 3.3% | (1 heteroz) | 0 |
| WDR36 | nt 191 T > C | Exon 1 | L25P | | 5.8% | (1 homoz) | 0 |
| WDR36 | nt 6579 C > T | Exon 4 | A163V | | 3.2% | (1 heteroz) | 0 |
| WDR36 | nt 17971 A > G | Intron 11 | | | 8.6% | (1.5) | 0 |
| WDR36 | nt 17980 G > A | Intron 11 | loss of PESS | | 3.9% | (1 heteroz) | 0 |
| PWP2 | nt 14867 T > A | Exon 14 | F551I | rs17856422 | 3.7% | (1 heteroz) | 0 |
| WDR3 | nt 20197 T > G | Intron 14 | loss of PESS | rs41276602 | 2.9% | (1 heteroz) | 0 |

Nucleotide positions are based on gene sequences stored in NCBI databases. Loss of PESS (putative exonic splicing silencer) and PESE (putative exonic splicing enhancer) motifs were predicted by use of Analyzer Splice Tool software (http://ast.bioinfo.tau.ac.il/SpliceSiteFrame.htm). Estimated frequencies in patients (P) and controls (C) are indicated.

### Validation of the Variants

The variants identified in patients were genotyped in each individual by Allele Specific Oligonucleotide—Polymerase Chain Reaction (ASO-PCR), with the exception for nt 8216 A>G in intron 3 of the UTP18 and nt 17971 A>G in intron 12 of the WDR36 because they weren't predicted to be involved in splicing sites or regulatory motifs. In ASO-PCR oligonucleotide primers are designed such that they are complementary to the wild type or mutant sequence and each one is used in conjunction with a common primer. Because DNA polymerase lacks a 3′ exonuclease activity, it is unable to repair a single-base mismatch between the primer and the template. Thus, the primer will or will not be extended depending on which alternative single-base polymorphism is present in the target sequence. Hence, under the appropriately stringent conditions, only target DNA exactly complementary to the primer will be amplified.

Allele-specific oligonucleotide primers with the correspondingly different bases at the 3′end and common primer were designed for each variant by use of OligoExplorer and OligoAnalyzer softwares. The reactions were accurately optimized and the 11 variants genotyped in all 17 patients. Eight of these 11 variants were confirmed (Table III and FIG. 1), the other 3 correspond to wild type genotypes. To validate this genotyping approach, all genotypes were confirmed by Sanger sequencing. In the identified 8 variants, the previously estimated frequencies were confirmed, except for nt 3895 G>A in TBL3 gene as shown in table II.

In order to increase the significance of the results and given the limitation in involving more patients in this study, the 8 identified alterations were also genotyped in a total of 95 healthy individuals by ASO-PCR. The results are given in Table III and compared with the glaucoma group.

TABLE III
Genotyping of the previously selected and identified alterations in patients (n = 17) and controls (n = 95).

| | | Frequency | | Genotyping | | | Genotyping | |
|---|---|---|---|---|---|---|---|---|
| Gene | Variant | by 454 | | patients (ASO-PCR) n17 | | ID Patients | controls n95 | ID Controls |
| TBL3 | nt 3895 G > A | 6.7% | (1 HM) | 11.8% | (1 HM; 2 HT) | 14; 5, 11 | 11.1% (2 HM; | 2, 44; 5, 8, 12, 13, 15, |
| | | | | | | | 17 HT) | 16, 23, 25, 27, 32, 34, |
| | | | | | | | | 35, 40, 41, 43, 46, 64 |
| UTP20 | nt 10156 A > C | 3.0% | (1 HT) | 2.9% | (1 HT) | 16 | 1.1% (2 HT) | 31, 60 |
| UTP20 | nt 73119 T > C | 2.9% | (1 HT) | 2.9% | (1 HT) | 15 | 2.1% (4 HT) | 67, 56, 76, 77 |
| WDR36 | nt 191 T > C | 5.8% | (1 HM) | 5.9% | (2 HT) | 5, 6 | 0.5% (1 HT) | 11 |
| WDR36 | nt 6579 C > T | 3.2% | (1 HT) | 2.9% | (1 HT) | 10 | All wild type | |
| WDR36 | nt 17980 G > A | 3.9% | (1 HT) | 2.9% | (1 HT) | 6 | All wild type | |
| PWP2 | nt 14867 T > A | 3.7% | (1 HT) | 2.9% | (1 HT) | 17 | All wild type | |
| WDR3 | nt 20197 T > G | 2.9% | (1 HT) | 2.9% | (1 HT) | 1 | 1.1% (2 HT) | 17, 57 |

HM: homozygous, HT: heterozygous, ID: internal number to identify each individual.

In TBL3 an intronic variant, nt 3895 G>A, predicted to lead to loss of a PESS motif was detected in 1 patient in the homozygous state and in 2 in the heterozygous state (11.8%). This variant was also found in 2 controls in the homozygous state and in 17 in the heterozygous state (11.1%).

In UTP20 an intronic alteration, nt 10156 A>C, predicted to be associated with loss of a PESS motif was found in 1 patient (2.9%) and in 2 controls (1.0%) in the heterozygous state. In the same gene, nt 73119 T>C, another intronic variant predicted to be related with loss of a PESE motif was found in 1 patient (2.9%) and 4 controls (2.1%) in the heterozygous state.

In WDR36 an exonic alteration, nt 191 T>C, that result in the conversion of leucine to a proline in codon 25 (L25P) was detected in 2 patients (5.9%) and 1 control (0.5%) in the heterozygous state. In the same gene, another one, nt 6579 C>T, causing an alanine to valine change at codon 163 (A163V) was found in 1 patient in the heterozygous state (2.9%). Still in this gene, nt 17980 G>A, an intronic variant predicted to be associated with loss of a PESS motif was detected in 1 patient in the heterozygous state (2.9%). None of these two changes were identified in undiseased individuals.

In PWP2, nt 14867 T>A, changing a phenylalanine to a isoleucine in codon 551 (F551I) was detect in 1 patient in the heterozygous state (2.9%). This one wasn't also detected in controls.

In WDR3 an intronic variant, nt 2019 T>G, resulting in loss of a PESS motif was detected in 1 patient (2.9%) and 2 controls (0.9%) in the heterozygous state.

Patient 5 is carrier of two heterozygous alterations, nt 3895 G>A in TBL3 gene and nt 191 T>C in WDR36 gene. Patient 6 has two alterations, nt 191 T>C and nt 17980 G>A, in the heterozygous state in WDR 36 gene.

Overall, four (WDR 36, PWP2, UTP20 and WDR 3) genes encoding polypeptide components of the claimed protein complex are potentially associated with the disease (thought this conclusion preferably needs to be confirmed in a larger group of patients).

### CONCLUSIONS

From this analysis the inventors have concluded that:
From the 18 high confident selected variants, 11 were further analyzed and 8 were validated in 5 genes of the protein complex associated with the disease.
Three of these 8 variants, in WDR36 and PWP2 genes, were only identified in patients.
The others, except for TBL3, were always found in patients in higher frequency.
The alteration in TBL3, although has been identified in patients and controls in the same frequency, occurs simultaneously with the heterozygous nt 191 T>C change in WDR36 gene of patient 5. Thus, a familial study of this patient could also imply TBL3 gene in the disease.
Except for TBL3, homozygous alterations weren't identified, however the association of both nt 191 T>C and nt 17980 G>A heterozygous changes with disease phenotype, in patient 6, suggest the involvement of heterozygous alterations in the disease.
Contrary to the patient population, in controls there aren't variants occurring simultaneously in the same individual.

### PROTEIN SEQUENCES

```
SEQ ID No: 1: UTP20_Human (http://beta.uniprot.org/uniprot/O75691)
                10 20 30 40 50 60
MKTKPVSHKT ENTYRFLTFA ERLGNVNIDI IHRIDRTASY EEEVETYFFE GLLKWRELNL
                70 80 90 100 110 120
TEHFGKFYKE VIDKCQSFNQ LVYHQNEIVQ SLKTHLQVKN SFAYQPLLDL VVQLARDLQM
              130 140 150 160 170 180
DFYPHFPEFF LTITSILETQ DTELLEWAFT SLSYLYKYLW RLMVKDMSSI YSMYSTLLAH
              190 200 210 220 230 240
KKLHIRNFAA ESFTFLMRKV SDKNALFNLM FLDLDKHPEK VEGVGQLLFE MCKGVRNMFH
              250 260 270 280 290 300
SCTGQAVKLI LRKLGPVTET ETQLPWMLIG ETLKNMVKST VSYISKEHFG TFFECLQESL
              310 320 330 340 350 360
LDLHTKVTKT NCCESSEQIK RLLETYLILV KHGSGTKIPT PADVCKVLSQ TLQVASLSTS
              370 380 390 400 410 420
CWETLLDVIS ALILGENVSL PETLIKETIE KIFESRFEKR LIFSFSEVMF AMKQFEQLFL
              430 440 450 460 470 480
PSFLSYIVNC FLIDDAVVKD EALAILAKLI LNKAAPPTAG SMAIEKYPLV FSPQMVGFYI
              490 500 510 520 530 540
KQKKTRSKGR NEQFPVLDHL LSIIKLPPNK DTTYLSQSWA ALVVLPHIRP LEKEKVIPLV
              550 560 570 580 590 600
TGFIEALFMT VDKGSFGKGN LFVLCQAVNT LLSLEESSEL LHLVPVERVK NLVLTFPLEP
              610 620 630 640 650 660
SVLLLTDLYY QRLALCGCKG PLSQEALMEL FPKLQANIST GVSKIRLLTI RILNHFDVQL
              670 680 690 700 710 720
PESMEDDGLS ERQSVFAILR QAELVPATVN DYREKLLHLR KLRHDVVQTA VPDGPLQEVP
              730 740 750 760 770 780
LRYLLGMLYI NFSALWDPVI ELISSHAHEM ENKQFWKVYY EHLEKAATHA EKELQNDMTD
              790 800 810 820 830 840
EKSVGDESWE QTQEGDVGAL YHEQLALKTD CQERLDHTNF RFLLWRALTK FPERVEPRSR
              850 860 870 880 890 900
ELSPLFLRFI NNEYYPADLQ VAPTQDLRRK GKGMVAEEIE EEPAAGDDEE LEEEAVPQDE
              910 920 930 940 950 960
SSQKKKTRRA AAKQLIAHLQ VFSKFSNPRA LYLESKLYEL YLQLLLHQDQ MVQKITLDCI
              970 980 990 1000 1010 1020
MTYKHPHVLP YRENLQRLLE DRSFKEEIVH FSISEDNAVV KTAHRADLFP ILMRILYGRM
            1030 1040 1050 1060 1070 1080
KNKTGSKTQG KSASGTRMAI VLRFLAGTQP EEIQIFLDLL FEPVRHFKNG ECHSAVIQAV
            1090 1100 1110 1120 1130 1140
EDLDLSKVLP LGRQHGILNS LEIVLKNISH LISAYLPKIL QILLCMTATV SHILDQREKI
            1150 1160 1170 1180 1190 1200
QLRFINPLKN LRRLGIKMVT DIFLDWESYQ FRTEEIDAVF HGAVWPQISR LGSESQYSPT
            1210 1220 1230 1240 1250 1260
PLLKLISIWS RNARYFPLLA KQKPGHPECD ILTNVFAILS AKNLSDATAS IVMDIVDDLL
            1270 1280 1290 1300 1310 1320
NLPDFEPTET VLNLLVTGCV YPGIAENIGE SITIGGRLIL PHVPAILQYL SKTTISAEKV
            1330 1340 1350 1360 1370 1380
KKKKNRAQVS KELGILSKIS KFMKDKEQSS VLITLLLPFL HRGNIAEDTE VDILVTVQNL
            1390 1400 1410 1420 1430 1440
LKHCVDPTSF LKPIAKLFSV IKNKLSRKLL CTVFETLSDF ESGLKYITDV VKLNAFDQRH
            1450 1460 1470 1480 1490 1500
LDDINFDVRF ETFQTITSYI KEMQIVDVNY LIPVMHNCFY NLELGDMSLS DNASMCLMSI
            1510 1520 1530 1540 1550 1560
IKKLPALNVT EKDYREIIHR SLLEKLRKGL KSQTESIQQD YTTILSCLIQ TFPNQLEFKD
            1570 1580 1590 1600 1610 1620
LVQLTHYHDP EMDFFENMKH IQIHRRARAL KKLAKQLMEG KVVLSSKSLQ NYIMPYAMTP
            1630 1640 1650 1660 1670 1680
IFDEKMLKHE NITTAATEII GAICKHLSWS AYMYYLKHFI HVLQTGQINQ KLGVSLLVIV
            1690 1700 1710 1720 1730 1740
LEAFHFDHKT LEEQMGKIEN EENAIEAIEL PEPEAMELER VDEEEKEYTC KSLSDNGQPG
            1750 1760 1770 1780 1790 1800
TPDPADSGGT SAKESECITK PVSFLPQNKE EIERTIKNIQ GTITGDILPR LHKCLASTTK
            1810 1820 1830 1840 1850 1860
REEEHKLVKS KVVNDEEVVR VPLAFAMVKL MQSLPQEVME ANLPSILLKV CALLKNRAQE
            1870 1880 1890 1900 1910 1920
IRDIARSTLA KIIEDLGVHF LQYVLKELQT TLVRGYQVHV LTFTVHMLLQ GLTNKLQVGD
            1930 1940 1950 1960 1970 1980
LDSCLDIMIE IFNHELFGAV AEEKEVKQIL SKVMEARRSK SYDSYEILGK FVGKDQVTKL
            1990 2000 2010 2020 2030 2040
ILPLKEILQN TTSLKLARKV HETLRRITVG LIVNQEMTAE SILLLSYGLI SENLPLLTEK
            2050 2060 2070 2080 2090 2100
EKNPVAPAPD PRLPPQSCLL LPPTPVRGGQ KAVVSRKTNM HIFIESGLRL LHLSLKTSKI
            2110 2120 2130 2140 2150 2160
KSSGECVLEM LDPFVSLLID CLGSMDVKVI TGALQCLIWV LRFPLPSIET KAEQLTKHLF
            2170 2180 2190 2200 2210 2220
LLLKDYAKLG AARGQNFHLV VNCFKCVTIL VKKVKSYQIT EKQLQVLLAY AEEDIYDTSR
            2230 2240 2250 2260 2270 2280
QATAFGLLKA ILSRKLLVPE IDEVMRKVSK LAVSAQSEPA RVQCRQVFLK YILDYPLGDK
            2290 2300 2310 2320 2330 2340
LRPNLEFMLA QLNYEHETGR ESTLEMIAYL FDTFPQGLLH ENCGMFFIPL CLMTINDDSA
            2350 2360 2370 2380 2390 2400
TCKKMASMTI KSLLGKISLE KKDWLFDMVT TWFGAKKRLN RQLAALICGL FVESEGVDFE
            2410 2420 2430 2440 2450 2460
KRLGTVLPVI EKEIDPENFK DIMEETEEKA ADRLLFSFLT LITKLIKECN IIQFTKPAET
            2470 2480 2490 2500 2510 2520
LSKIWSHVHS HLRHPHNWVW LTAAQIFGLL FASCQPEELI QKWNTKKTKK HLPEPVAIKF
            2530 2540 2550 2560 2570 2580
LASDLDQKMK SISLASCHQL HSKFLDQSLG EQVVKNLLFA AKVLYLLELY CEDKQSKIKE
            2590 2600 2610 2620 2630 2640
DLEEQEALED GVACADEKAE SDGEEKEEVK EELGRPATLL WLIQKLSRIA KLEAAYSPRN
            2650 2660 2670 2680 2690 2700
PLKRTCIFKF LGAVAMDLGI DKVKPYLPMI IAPLFRELNS TYSEQDPLLK NLSQEIIELL
            2710 2720 2730 2740 2750 2760
KKLVGLESFS LAFASVQKQA NEKRALRKKR KALEFVTNPD IAAKKKMKKH KNKSEAKKRK
            2770 2780
IEFLRPGYKA KRQKSHSLKD LAMVE
SEQ ID No: 2: PWP2_Human (http://beta.uniprot.org/uniprot/Q15269)
                10 20 30 40 50 60
MKFAYRFSNL LGTVYRRGNL NFTCDGNSVI SPVGNRVTVF DLKNNKSDTL PLATRYNVKC
                70 80 90 100 110 120
VGLSPDGRLA IIVDEGGDAL LVSLVCRSVL HHFHFKGSVH SVSFSPDGRK FVVTKGNIAQ
              130 140 150 160 170 180
MYHAPGKKRE FNAFVLDKTY FGPYDETTCI DWTDDSRCFV VGSKDMSTWV FGAERWDNLI
              190 200 210 220 230 240
YYALGGHKDA IVACFFESNS LDLYSLSQDG VLCMWQCDTP PEGLRLKPPA GWKADLLQRE
              250 260 270 280 290 300
EEEEEEEDQE GDRETTIRGK ATPAEEEKTG KVKYSRLAKY FFNKEGDFNN LTAAAFHKKS
              310 320 330 340 350 360
HLLVTGFASG IFHLHELPEF NLIHSLSISD QSIASVAINS SGDWIAFGCS GLGQLLVWEW
              370 380 390 400 410 420
QSESYVLKQQ GHFNSMVALA YSPDGQYIVT GGDDGKVKVW NTLSGFCFVT FTEHSSGVTG
              430 440 450 460 470 480
VTFTATGYVV VTSSMDGTVR AFDLHRYRNF RTFTSPRPTQ FSCVAVDASG EIVSAGAQDS
              490 500 510 520 530 540
FEIFVWSMQT GRLLDVLSGH EGPISGLCFN PMKSVLASAS WDKTVRLWDM FDSWRTKETL
              550 560 570 580 590 600
ALTSDALAVT FRPDGAELAV ATLNSQITFW DPENAVQTGS IEGRHDLKTG RKELDKITAK
              610 620 630 640 650 660
HAAKGKAFTA LCYSADGHSI LAGGMSKFVC IYHVREQILM KRFEISCNLS LDAMEEFLNR
              670 680 690 700 710 720
RKMTEFGNLA LIDQDAGQED GVAIPLPGVR KGDMSSRHFK PEIRVTSLRF SPTGRCWAAT
              730 740 750 760 770 780
TTEGLLIYSL DTRVLFDPFE LDTSVTPGRV REALRQQDFT RAILMALRLN ESKLVQEALE
              790 800 810 820 830 840
AVPRGEIEVV TSSLPELYVE KVLEFLASSF EVSRHLEFYL LWTHKLLMLH GQKLKSRAGT
              850 860 870 880 890 900
LLPVIQFLQK SIQRHLDDLS KLCSWNHYNM QYALAVSKQR GTKRSLDPLG SEEEAEASED
              910
DSLHLLGGGG RDSEEEMLA
SEQ ID No: 3: WDR46_Human (http://beta.uniprot.org/uniprot/O15213)
                10 20 30 40 50 60
METAPKPGKD VPPKKDKLQT KRKKPRRYWE EETVPTTAGA SPGPPRNKKN RELRPQRPKN
                70 80 90 100 110 120
AYILKKSRIS KKPQVPKKPR EWKNPESQRG LSGAQDPFPG PAPVPVEVVQ KFCRIDKSRK
              130 140 150 160 170 180
LPHSKAKTRS RLEVAEAEEE ETSIKAARSE LLLAEEPGFL EGEDGEDTAK ICQADIVEAV
              190 200 210 220 230 240
DIASAAKHFD LNLRQFGPYR LNYSRTGRHL AFGGRRGHVA ALDWVTKKLM CEINVMEAVR
              250 260 270 280 290 300
DIRFLHSEAL LAVAQNRWLH IYDNQGIELH CIRRCDRVTR LEFLPFHFLL ATASETGFLT
              310 320 330 340 350 360
YLDVSVGKIV AALNARAGRL DVMSQNPYNA VIHLGHSNGT VSLWSPAMKE PLAKILCHRG
              370 380 390 400 410 420
GVRAVAVDST GTYMATSGLD HQLKIFDLRG TYQPLSTRTL PHGAGHLAFS QRGLLVAGMG
              430 440 450 460 470 480
DVVNIWAGQG KASPPSLEQP YLTHRLSGPV HGLQFCPFED VLGVGHTGGI TSMLVPGAGE
              490 500 510 520 530 540
PNFDGLESNP YRSRKQRQEW EVKALLEKVP AELICLDPRA LAEVDVISLE QGKKEQIERL
              550 560 570 580 590 600
GYDPQAKAPF QPKPKQKGRS STASLVKRKR KVMDEEHRDK VRQSLQQQHH KEAKAKPTGA
              610
RPSALDRFVR
SEQ ID No: 4: UTP18_Human (http://beta.uniprot.org/uniprot/Q9Y5J1)
                10 20 30 40 50 60
MPPERRRRMK LDRRTGAKPK RKPGMRPDWK AGAGPGGPPQ KPAPSSQRKP PARPSAAAAA
                70 80 90 100 110 120
IAVAAAEEER RLRQRNRLRL EEDKPAVERC LEELVFGDVE NDEDALLRRL RGPRVQEHED
              130 140 150 160 170 180
SGDSEVENEA KGNFPPQKKP VWVDEEDEDE EMVDMMNNRF RKDMMKNASE SKLSKDNLKK
              190 200 210 220 230 240
RLKEEFQHAM GGVPAWAETT KRKTSSDDES EEDEDDLLQR TGNFISTSTS LPRGILKMKN
              250 260 270 280 290 300
CQHANAERPT VARISSVQFH PGAQIVMVAG LDNAVSLFQV DGKTNPKIQS IYLERFPIFK
              310 320 330 340 350 360
ACFSANGEEV LATSTHSKVL YVYDMLAGKL IPVHQVRGLK EKIVRSFEVS PDGSFLLING
              370 380 390 400 410 420
IAGYLHLLAM KTKELIGSMK INGRVAASTF SSDSKKVYAS SGDGEVYVWD VNSRKCLNRF
              430 440 450 460 470 480
VDEGSLYGLS IATSRNGQYV ACGSNCGVVN IYNQDSCLQE TNPKPIKAIM NLVTGVTSLT
              490 500 510 520 530 540
FNPTTEILAI ASEKMKEAVR LVHLPSCTVF SNFPVIKNKN ISHVHTMDFS PRSGYFALGN
              550
EKGKALMYRL HHYSDF
SEQ ID No: 5: MPP10_Human (http://beta.uniprot.org/uniprot/O00566)
                10 20 30 40 50 60
MAPQVWRRRT LERCLTEVGK ATGRPECFLT IQEGLASKFT SLTKVLYDFN KILENGRIHG
                70 80 90 100 110 120
SPLQKLVIEN FDDEQIWQQL ELQNEPILQY FQNAVSETIN DEDISLLPES EEQEREEDGS
              130 140 150 160 170 180
EIEADDKEDL EDLEEEEVSD MGNDDPEMGE RAENSSKSDL RKSPVFSDED SDLDFDISKL
              190 200 210 220 230 240
EQQSKVQNKG QGKPREKSIV DDKFFKLSEM EAYLENIEKE EERKDDNDEE EEDIDFFEDI
              250 260 270 280 290 300
DSDEDEGGLF GSKKLKSGKS SRNLKYKDFF DPVESDEDIT NVHDDELDSN KEDDEIAEEE
              310 320 330 340 350 360
AEELSISETD EDDDLQENED NKQHKESLKR VTFALPDDAE TEDTGVLNVK KNSDEVKSSF
              370 380 390 400 410 420
EKRQEKMNEK IASLEKELLE KKPWQLQGEV TAQKRPENSL LEETLHFDHA VRMAPVITEE
              430 440 450 460 470 480
TTLQLEDIIK QRIRDQAWDD VVRKEKPKED AYEYKKRLTL DHEKSKLSLA EIYEQEYIKL
              490 500 510 520 530 540
NQQKTAEEEN PEHVEIQKMM DSLFLKLDAL SNFHFIPKPP VPEIKVVSNL PAITMEEVAP
              550 560 570 580 590 600
VSVSDAALLA PEEIKEKNKA GDIKTAAEKT ATDKKRERRK KKYQKRMKIK EKEKRRKLLE
              610 620 630 640 650 660
KSSVDQAGKY SKTVASEKLK QLTKTGKASF IKDEGKDKAL KSSQAFFSKL QDQVKMQIND
              670 680
AKKTEKKKKK RQDISVHKLK L
SEQ ID No: 6: WDR3_Human (http://beta.uniprot.org/uniprot/Q9UNX4)
                10 20 30 40 50 60
MGLTKQYLRY VASAVFGVIG SQKGNIVFVT LRGEKGRYVA VPACEHVFIW DLRKGEKILI
                70 80 90 100 110 120
LQGLKQEVTC LCPSPDGLHL AVGYEDGSIR IFSLLSGEGN VTFNGHKAAI TTLKYDQLGG
              130 140 150 160 170 180
RLASGSKDTD IIVWDVINES GLYRLKGHKD AITQALFLRE KNLLVTSGKD TMVKWWDLDT
              190 200 210 220 230 240
QHCFKTMVGH RTEVWGLVLL SEEKRLITGA SDSELRVWDI AYLQEIEDPE EPDPKKIKGS
              250 260 270 280 290 300
SPGIQDTLEA EDGAFETDEA PEDRILSCRK AGSIMREGRD RVVNLAVDKT GRILACHGTD
              310 320 330 340 350 360
SVLELFCILS KKEIQKKMDK KMKKARKKAK LHSSKGEEED PEVNVEMSLQ DEIQRVTNIK
              370 380 390 400 410 420
TSAKIKSFDL IHSPHGELKA VFLLQNNLVE LYSLNPSLPT PQPVRTSRIT IGGHRSDVRT
              430 440 450 460 470 480
LSFSSDNIAV LSAAADSIKI WNRSTLQCIR TMTCEYALCS FFVPGDRQVV IGTKTGKLQL
              490 500 510 520 530 540
YDLASGNLLE TIDAHDGALW SMSLSPDQRG FVTGGADKSV KFWDFELVKD ENSTQKRLSV
              550 560 570 580 590 600
KQTRTLQLDE DVLCVSYSPN QKLLAVSLLD CTVKIFYVDT LKFFLSLYGH KLPVICMDIS
              610 620 630 640 650 660
HDGALIATGS ADRNVKIWGL DFGDCHKSLF AHDDSVMYLQ FVPKSHLFFT AGKDHKIKQW
              670 680 690 700 710 720
DADKFEHIQT LEGHHQEIWC LAVSPSGDYV VSSSHDKSLR LWERTREPLI LEEEREMERE
              730 740 750 760 770 780
AEYEESVAKE DQPAVPGETQ GDSYFTGKKT IETVKAAERI MEAIELYREE TAKMKEHKAI
              790 800 810 820 830 840
CKAAGKEVPL PSNPILMAYG SISPSAYVLE IFKGIKSSEL EESLLVLPFS YVPDILKLFN
              850 860 870 880 890 900
EFIQLGSDVE LICRCLFFLL RIHFGQITSN QMLVPVIEKL RETTISKVSQ VRDVIGFNMA
              910 920 930 940
GLDYLKRECE AKSEVMFFAD ATSHLEEKKR KRKKREKLIL TLT
SEQ ID No: 7: TBL3_Human (http://beta.uniprot.org/uniprot/Q12788)
                10 20 30 40 50 60
MAFDPTSTLL ATGGCDGAVR VWDIVRHYGT HHFRGSPGVV HLVAFHPDPT RLLLFSSATD
                70 80 90 100 110 120
AAIRVWSLQD RSCLAVLTAH YSAVTSLAFS ADGHTMLSSG RDKICIIWDL QSCQATRTVP
              130 140 150 160 170 180
VFESVEAAVL LPEEPVSQLG VKSPGLYFLT AGDQGTLRVW EAASGQCVYT QAQPPGPGQE
              190 200 210 220 230 240
LTHCTLAHTA GVVLTATADH NLLLYEARSL RLQKQFAGYS EEVLDVRFLG PEDSHVVVAS
              250 260 270 280 290 300
NSPCLKVFEL QTSACQILHG HTDIVLALDV FRKGWLFASC AKDQSVRIWR MNKAGQVMCV
              310 320 330 340 350 360
AQGSGHTHSV GTVCCSRLKE SFLVTGSQDC TVKLWPLPKA LLSKNTAPDN GPILLQAHTT
              370 380 390 400 410 420
QRCHDKDINS VAIAPNDKLL ATGSQDRTAK LWALPQCQLL GVFSGHRVAS GASSSLPWTR
              430 440 450 460 470 480
CWPRPQLMAP SSSGHSRTSA VSRHLRGTML LCLKVAFVSR GTQLLSSGSD GLVKLWTIKN
              490 500 510
NECVRTLDAH EDKVWGLQAG WTTTPSLGPV TPESSSGRM
SEQ ID No: 8: WDR36_Human (http://beta.uniprot.org/uniprot/Q8NI36)
                10 20 30 40 50 60
MCCTEGSLRK RDSQRAPEAV LCLQLWQRTV PLDTLKGLGT CFPSGPELRG AGIAAAMERA
                70 80 90 100 110 120
SERRTASALF AGFRALGLFS NDIPHVVRFS ALKRRFYVTT CVGKSFHTYD VQKLSLVAVS
              130 140 150 160 170 180
NSVPQDICCM AADGRLVFAA YGNVFSAFAR NKEIVHTFKG HKAEIHFLQP FGDHIISVDT
              190 200 210 220 230 240
DGILIIWHIY SEEEYLQLTF DKSVFKISAI LHPSTYLNKI LLGSEQGSLQ LWNVKSNKLL
              250 260 270 280 290 300
YTFPGWKVGV TALQQAPAVD VVAIGLMSGQ VIIHNIKFNE TLMKFRQDWG PITSISFRTD
              310 320 330 340 350 360
GHPVMAAGSP CGHIGLWDLE DKKLINQMRN AHSTAIAGLT FLHREPLLVT NGADNALRIW
              370 380 390 400 410 420
IFDGPTGEGR LLRFRMGHSA PLTNIRYYGQ NGQQILSASQ DGTLQSFSTV HEKFNKSLGH
              430 440 450 460 470 480
GLINKKRVKR KGLQNTMSVR LPPITKFAAE EARESDWDGI IACHQGKLSC STWNYQKSTI
              490 500 510 520 530 540
GAYFLKPKEL KKDDITATAV DITSCGNFAV IGLSSGTVDV YNMQSGIHRG SFGKDQAHKG
              550 560 570 580 590 600
SVRGVAVDGL NQLTVTTGSE GLLKFWNFKN KILIHSVSLS SSPNIMLLHR DSGILGLALD
              610 620 630 640 650 660
DFSISVLDIE TRKIVREFSG HQGQINDMAF SPDGRWLISA AMDCSIRTWD LPSGCLIDCF
              670 680 690 700 710 720
LLDSAPLNVS MSPTGDFLAT SHVDHLGIYL WSNISLYSVV SLRPLPADYV PSIVMLPGTC
              730 740 750 760 770 780
QTQDVEVSEE TVEPSDELIE YDSPEQLNEQ LVTLSLLPES RWKNLLNLDV IKKKNKPKEP
              790 800 810 820 830 840
PKVPKSAPFF IPTIPGLVPR YAAPEQNNDP QQSKVVNLGV LAQKSDFCLK LEEGLVNNKY
              850 860 870 880 890 900
DTALNLLKES GPSGIETELR SLSPDCGGSI EVMQSFLKMI GMMLDRKRDF ELAQAYLALF
              910 920 930 940 950
LKLHLKMLPS EPVLLEEITN LSSQVEENWT HLQSLFNQSM CILNYLKSAL L
SEQ ID No: 9: NOC4L_Human (http://beta.uniprot.org/uniprot/Q9BVI4)
                10 20 30 40 50 60
MEREPGAAGV RRALGRRLEA VLASRSEANA VFDILAVLQS EDQEEIQEAV RTCSRLFGAL
                70 80 90 100 110 120
LERGELFVGQ LPSEEMVMTG SQGATRKYKV WMRHRYHSCC NRLGELLGHP SFQVKELALS
              130 140 150 160 170 180
ALLKFVQLEG AHPLEKSKWE GNYLFPRELF KLVVGGLLSP EEDQSLLLSQ FREYLDYDDT
              190 200 210 220 230 240
RYHTMQAAVD AVARVTGQHP EVPPAFWNNA FTLLSAVSLP RREPTVSSFY VKRAELWDTW
              250 260 270 280 290 300
KVAHLKEHRR VFQAMWLSFL KHKLPLSLYK KVLLIVHDAI LPQLAQPTLM IDFLTRACDL
              310 320 330 340 350 360
GGALSLLALN GLFILIHKHN LEYPDFYRKL YGLLDPSVFH VKYRARFFHL ADLFLSSSHL
              370 380 390 400 410 420
PAYLVAAFAK RLARLALTAP PEALLMVLPF ICNLLRRHPA CRVLVHRPHG PELDADPYDP
              430 440 450 460 470 480
GEEDPAQSRA LESSLWELQA LQRHYHPEVS KAASVINQAL SMPEVSIAPL LELTAYEIFE
              490 500 510
RDLKKKGPEP VPLEFIPAQG LLGRPGELCA QHFTLS
SEQ ID No: 10: YBA4_Yeast (http://beta.uniprot.org/uniprot/P35194)
                10 20 30 40 50 60
MAKQRQTTKS SKRYRYSSFK ARIDDLKIEP ARNLEKRVHD YVESSHFLAS FDQWKEINLS
                70 80 90 100 110 120
AKFTEFAAEI EHDVQTLPQI LYHDKKIFNS LVSFINFHDE FSLQPLLDLL AQFCHDLGPD
              130 140 150 160 170 180
FLKFYEEAIK TLINLLDAAI EFESSNVFEW GFNCLAYIFK YLSKFLVKKL VLTCDLLIPL
              190 200 210 220 230 240
LSHSKEYLSR FSAEALSFLV RKCPVSNLRE FVRSVFEKLE GDDEQTNLYE GLLILFTESM
              250 260 270 280 290 300
TSTQETLHSK AKAIMSVLLH EALTKSSPER SVSLLSDIWM NISKYASIES LLPVYEVMYQ
              310 320 330 340 350 360
DFNDSLDATN IDRILKVLTT IVFSESGRKI PDWNKITILI ERIMSQSENC ASLSQDKVAF
              370 380 390 400 410 420
LFALFIRNSD VKTLTLFHQK LFNYALTNIS DCFLEFFQFA LRLSYERVFS FNGLKFLQLF
              430 440 450 460 470 480
LKKNWQSQGK KIALFFLEVD DKPELQKVRE VNFPEEFILS IRDFFVTAEI NDSNDLFEIY
              490 500 510 520 530 540
WRAIIFKYSK LQNTEIIIPL LERIFSTFAS PDNFTKDMVG TLLKIYRKED DASGNNLLKT
              550 560 570 580 590 600
ILDNYENYKE SLNFLRGWNK LVSNLHPSES LKGLMSHYPS LLLSLTDNFM LPDGKIRYET
              610 620 630 640 650 660
LELMKTLMIL QGMQVPDLLS SCMVIEEIPL TLQNARDLTI RIKNVGAEFG KTKTDKLVSS
              670 680 690 700 710 720
FFLKYLFGLL TVRFSPVWTG VFDTLPNVYT KDEALVWKLV LSFIKLPDEN QNLDYYQPLL
              730 740 750 760 770 780
EDGANKVLWD SSVVRLRDTI DTFSHIWSKY STQNTSIIST TIERRGNTTY PILIRNQALK
              790 800 810 820 830 840
VMLSIPQVAE NHFVDIAPFV YNDFKTYKDE EDMENERVIT GSWTEVDRNV FLKTLSKFKN
              850 860 870 880 890 900
IKNVYSATEL HDHLMVLLGS RNTDVQKLAL DALLAYKNPT LNKYRDNLKN LLDDTLFKDE
              910 920 930 940 950 960
ITTFLTENGS QSIKAEDEKV VMPYVLRIFF GRAQVPPTSG QKRSRKIAVI SVLPNFKKPY
              970 980 990 1000 1010 1020
INDFLSLASE RLDYNYFFGN SHQINSSKAT LKTIRRMTGF VNIVNSTLSV LRTNFPLHTN
            1030 1040 1050 1060 1070 1080
SVLQPLIYSI AMAYYVLDTE STEEVHLRKM ASNLRQQGLK CLSSVFEFVG NTFDWSTSME
            1090 1100 1110 1120 1130 1140
DIYAVVVKPR ISHFSDENLQ QPSSLLRLFL YWAHNPSLYQ FLYYDEFATA TALMDTISNQ
            1150 1160 1170 1180 1190 1200
HVKEAVIGPI IEAADSIIRN PVNDDHYVDL VTLICTSCLK ILPSLYVKLS DSNSISTFLN
            1210 1220 1230 1240 1250 1260
LLVSITEMGF IQDDHVRSRL ISSLISILKG KLKKLQENDT QKILKILKLI VFNYNCSWSD
            1270 1280 1290 1300 1310 1320
IEELYTTISS LFKTFDERNL RVSLTELFIE LGRKVPELES ISKLVADLNS YSSSRMHEYD
            1330 1340 1350 1360 1370 1380
FPRILSTFKG LIEDGYKSYS ELEWLPLLFT FLHFINNKEE LALRTNASHA IMKFIDFINE
            1390 1400 1410 1420 1430 1440
KPNLNEASKS ISMLKDILLP NIRIGLRDSL EEVQSEYVSV LSYMVKNTKY FTDFEDMAIL
            1450 1460 1470 1480 1490 1500
LYNGDEEADF FTNVNHIQLH RRQRAIKRLG EHAHQLKDNS ISHYLIPMIE HYVFSDDERY
            1510 1520 1530 1540 1550 1560
RNIGNETQIA IGGLAQHMSW NQYKALLRRY ISMLKTKPNQ MKQAVQLIVQ LSVPLRETLR
            1570 1580 1590 1600 1610 1620
IVRDGAESKL TLSKFPSNLD EPSNFIKQEL YPTLSKILGT RDDETIIERM PIAEALVNIV
            1630 1640 1650 1660 1670 1680
LGLTNDDITN FLPSILTNIC QVLRSKSEEL RDAVRVTLGK ISIILGAEYL VFVIKELMAT
            1690 1700 1710 1720 1730 1740
LKRGSQIHVL SYTVHYILKS MHGVLKHSDL DTSSSMIVKI IMENIFGFAG EEKDSENYHT
            1750 1760 1770 1780 1790 1800
KVKEIKSNKS YDAGEILASN ISLTEFGTLL SPVKALLMVR INLRNQNKLS ELLRRYLLGL
            1810 1820 1830 1840 1850 1860
NHNSDSESES ILKFCHQLFQ ESEMSNSPQI PKKKVKDQVD EKEDFFLVNL ESKSYTINSN
            1870 1880 1890 1900 1910 1920
SLLLNSTLQK FALDLLRNVI TRHRSFLTVS HLEGFIPFLR DSLLSENEGV VISTLRILIT
            1930 1940 1950 1960 1970 1980
LIRLDFSDES SEIFKNCARK VLNIIKVSPS TSSELCQMGL KFLSAFIRHT DSTLKDTALS
            1990 2000 2010 2020 2030 2040
YVLGRVLPDL NEPSRQGLAF NFLKALVSKH IMLPELYDIA DTTREIMVTN HSKEIRDVSR
            2050 2060 2070 2080 2090 2100
SVYYQFLMEY DQSKGRLEKQ FKFMVDNLQY PTESGRQSVM ELINLIITKA NPALLSKLSS
            2110 2120 2130 2140 2150 2160
SFFLALVNVS FNDDAPRCRE MASVLISTML PKLENKDLEI VEKYIAAWLK QVDNASFLNL
            2170 2180 2190 2200 2210 2220
GLRTYKVYLK SIGFEHTIEL DELAIKRIRY ILSDTSVGSE HQWDLVYSAL NTFSSYMEAT
            2230 2240 2250 2260 2270 2280
ESVYKHGFKD IWDGIITCLL YPHSWVRQSA ANLVHQLIAN KDKLEISLTN LEIQTIATRI
            2290 2300 2310 2320 2330 2340
LHQLGAPSIP ENLANVSIKT LVNISILWKE QRTPFIMDVS KQTGEDLKYT TAIDYMVTRI
            2350 2360 2370 2380 2390 2400
GGIIRSDEHR MDSFMSKKAC IQLLALLVQV LDEDEVIAEG EKILLPLYGY LETYYSRAVD
            2410 2420 2430 2440 2450 2460
EEQEELRTLS NECLKILEDK LQVSDFTKIY TAVKQTVLER RKERRSKRAI LAVNAPQISA
            2470 2480 2490
DKKLRKHARS REKRKHEKDE NGYYQRRNKR KRA
SEQ ID No: 11: PWP2_Yeast (http://beta.uniprot.org/uniprot/P25635)
                10 20 30 40 50 60
MKSDFKFSNL LGTVYRQGNI TFSDDGKQLL SPVGNRVSVF DLINNKSFTF EYEHRKNIAA
                70 80 90 100 110 120
IDLNKQGTLL ISIDEDGRAI LVNFKARNVL HHFNFKEKCS AVKFSPDGRL FALASGRFLQ
              130 140 150 160 170 180
IWKTPDVNKD RQFAPFVRHR VHAGHFQDIT SLTWSQDSRF ILTTSKDLSA KIWSVDSEEK
              190 200 210 220 230 240
NLAATTFNGH RDYVMGAFFS HDQEKIYTVS KDGAVFVWEF TKRPSDDDDN ESEDDDKQEE
              250 260 270 280 290 300
VDISKYSWRI TKKHFFYANQ AKVKCVTFHP ATRLLAVGFT SGEFRLYDLP DFTLIQQLSM
              310 320 330 340 350 360
GQNPVNTVSV NQTGEWLAFG SSKLGQLLVY EWQSESYILK QQGHFDSTNS LAYSPDGSRV
              370 380 390 400 410 420
VTASEDGKIK VWDITSGFCL ATFEEHTSSV TAVQFAKRGQ VMFSSSLDGT VRAWDLIRYR
              430 440 450 460 470 480
NFRTFTGTER IQFNCLAVDP SGEVVCAGSL DNFDIHVWSV QTGQLLDALS GHEGPVSCLS
              490 500 510 520 530 540
FSQENSVLAS ASWDKTIRIW SIFGRSQQVE PIEVYSDVLA LSMRPDGKEV AVSTLKGQIS
              550 560 570 580 590 600
IFNIEDAKQV GNIDCRKDII SGRFNQDRFT AKNSERSKFF TTIHYSFDGM AIVAGGNNNS
              610 620 630 640 650 660
ICLYDVPNEV LLKRFIVSRN MALNGTLEFL NSKKMTEAGS LDLIDDAGEN SDLEDRIDNS
              670 680 690 700 710 720
LPGSQRGGDL STRKMRPEVR VTSVQFSPTA NAFAAASTEG LLIYSTNDTI LFDPFDLDVD
              730 740 750 760 770 780
VTPHSTVEAL REKQFLNALV MAFRLNEEYL INKVYEAIPI KEIPLVASNI PAIYLPRILK
              790 800 810 820 830 840
FIGDFAIESQ HIEFNLIWIK ALLSASGGYI NEHKYLFSTA MRSIQRFIVR VAKEVVNTTT
              850 860 870 880 890 900
DNKYTYRFLV STDGSMEDGA ADDDEVLLKD DADEDNEENE ENDVVMESDD EEGWIGFNGK
              910 920
DNKLPLSNEN DSSDEEENEK ELP
SEQ ID NO: 12: UTP7_Yeast (http://beta.uniprot.org/uniprot/P40055)
                10 20 30 40 50 60
MGHKKNGHRR QIKERENQNK FERSTYTNNA KNNHTQTKDK KLRAGLKKID EQYKKAVSSA
                70 80 90 100 110 120
AATDYLLPES NGYLEPENEL EKTFKVQQSE IKSSVDVSTA NKALDLSLKE FGPYHIKYAK
              130 140 150 160 170 180
NGTHLLITGR KGHVASMDWR KGQLRAELFL NETCHSATYL QNEQYFAVAQ KKYTFIYDHE
              190 200 210 220 230 240
GTELHRLKQH IEARHLDFLP YHYLLVTAGE TGWLKYHDVS TGQLVSELRT KAGPTMAMAQ
              250 260 270 280 290 300
NPWNAVMHLG HSNGTVSLWS PSMPEPLVKL LSARGPVNSI AIDRSGYYMA TTGADRSMKI
              310 320 330 340 350 360
WDIRNFKQLH SVESLPTPGT NVSISDTGLL ALSRGPHVTL WKDALKLSGD SKPCFGSMGG
              370 380 390 400 410 420
NPHRNTPYMS HLFAGNKVEN LGFVPFEDLL GVGHQTGITN LIVPGAGEAN YDALELNPFE
              430 440 450 460 470 480
TKKQRQEQEV RTLLNKLPAD TITLDPNSIG SVDKRSSTIR LNAKDLAQTT MDANNKAKTN
              490 500 510 520 530 540
SDIPDVKPDV KGKNSGLRSF LRKKTQNVID ERKLRVQKQL DKEKNIRKRN HQIKQGLISE
              550
DHKDVIEEAL SRFG
SEQ ID No: 13: UTP18_Yeast (http://beta.uniprot.org/uniprot/P40362)
                10 20 30 40 50 60
MTMATTAMNV SVPPPDEEEQ LLAKFVFGDT TDLQENLAKF NADFIFNEQE MDVEDQEDEG
                70 80 90 100 110 120
SESDNSEEDE AQNGELDHVN NDQLFFVDDG GNEDSQDKNE DTMDVDDEDD SSSDDYSEDS
              130 140 150 160 170 180
EEAAWIDSDD EKIKVPILVT NKTKKLRTSY NESKINGVHY INRLRSQFEK IYPRPKWVDD
              190 200 210 220 230 240
ESDSELDDEE DDEEEGSNNV INGDINALTK ILSTTYNYKD TLSNSKLLPP KKLDIVRLKD
              250 260 270 280 290 300
ANASHPSHSA IQSLSFHPSK PLLLTGGYDK TLRIYHIDGK TNHLVTSLHL VGSPIQTCTF
              310 320 330 340 350 360
YTSLSNQNQQ NIFTAGRRRY MHSWDLSLEN LTHSQTAKIE KFSRLYGHES TQRSFENFKV
              370 380 390 400 410 420
AHLQNSQTNS VHGIVLLQGN NGWINILHST SGLWLMGCKI EGVITDFCID YQPISRGKFR
              430 440 450 460 470 480
TILIAVNAYG EVWEFDLNKN GHVIRRWKDQ GGVGITKIQV GGGTTTTCPA LQISKIKQNR
              490 500 510 520 530 540
WLAVGSESGF VNLYDRNNAM TSSTPTPVAA LDQLTTTISN LQFSPDGQIL CMASRAVKDA
              550 560 570 580 590
LRLVHLPSCS VFSNWPTSGT PLGKVTSVAF SPSGGLLAVG NEQGKVRLWK LNHY
SEQ ID No: 14: MPP10_Yeast (http://beta.uniprot.org/uniprot/P47083)
                10 20 30 40 50 60
MSELFGVLKS NAGRIILKDP SATSKDVKAY IDSVINTCKN GSITKKAELD EITVDGLDAN
                70 80 90 100 110 120
QVWWQVKLVL DSIDGDLIQG IQELKDVVTP SHNLSDGSTL NSSSGEESEL EEAESVFKEK
              130 140 150 160
              170 180
QMLSADVSEI EEQSNDSLSE NDEEPSMDDE KTSAEAAREE FAEEKRISSG QDERHSSPDP
              190 200 210 220 230 240
YGINDKFFDL EKFNRDTLAA EDSNEASEGS EDEDIDYFQD MPSDDEEEEA IYYEDFFDKP
              250 260 270 280 290 300
TKEPVKKHSD VKDPKEDEEL DEEEHDSAMD KVKLDLFADE EDEPNAEGVG EASDKNLSSF
              310 320 330 340 350 360
EKQQIEIRKQ IEQLENEAVA EKKWSLKGEV KAKDRPEDAL LTEELEFDRT AKPVPVITSE
              370 380 390 400 410 420
VTESLEDMIR RRIQDSNFDD LQRRTLLDIT RKSQRPQFEL SDVKSSKSLA EIYEDDYTRA
              430 440 450 460 470 480
EDESALSEEL QKAHSEISEL YANLVYKLDV LSSVHFVPKP ASTSLEIRVE TPTISMEDAQ
              490 500 510 520 530 540
PLYMSNASSL APQEIYNVGK AEKDGEIRLK NGVAMSKEEL TREDKNRLRR ALKRKRSKAN
              550 560 570 580 590
LPNVNKRSKR NDVVDTLSKA KNITVINQKG EKKDVSGKTK KSRSGPDSTN IKL
SEQ ID No: 15: DIP2_Yeast (http://beta.uniprot.org/uniprot/Q12220)
                10 20 30 40 50 60
MVKSYQRFEQ AAAFGVIASN ANCVWIPASS GNSNGSGPGQ LITSALEDVN IWDIKTGDLV
                70 80 90 100 110 120
SKLSDGLPPG ASDARGAKPA ECTYLEAHKD TDLLAVGYAD GVIKVWDLMS KTVLLNFNGH
              130 140 150 160 170 180
KAAITLLQFD GTGTRLISGS KDSNIIVWDL VGEVGLYKLR SHKDSITGFW CQGEDWLIST
              190 200 210 220 230 240
SKDGMIKLWD LKTHQCIETH IAHTGECWGL AVKDDLLITT GTDSQVKIWK LDIENDKMGG
              250 260 270 280 290 300
KLTEMGIFEK QSKQRGLKIE FITNSSDKTS FFYIQNADKT IETFRIRKEE EIARGLKKRE
              310 320 330 340 350 360
KRLKEKGLTE EEIAKSIKES YSSFILHPFQ TIRSLYKIKS ASWTTVSSSK LELVLTTSSN
              370 380 390 400 410 420
TIEYYSIPYE KRDPTSPAPL KTHTIELQGQ RTDVRSIDIS DDNKLLATAS NGSLKIWNIK
              430 440 450 460 470 480
THKCIRTFEC GYALTCKFLP GGLLVILGTR NGELQLFDLA SSSLLDTIED AHDAAIWSLD
              490 500 510 520 530 540
LTSDGKRLVT GSADKTVKFW DFKVENSLVP GTKNKFLPVL KLHHDTTLEL TDDILCVRVS
              550 560 570 580 590 600
PDDRYLAISL LDNTVKVFFL DSMKFYLSLY GHKLPVLSID ISFDSKMIIT SSADKNIKIW
              610 620 630 640 650 660
GLDFGDCHKS LFAHQDSIMN VKFLPQSHNF FSCSKDAVVK YWDGEKFECI QKLYAHQSEV
              670 680 690 700 710 720
WALAVATDGG FVVSSSHDHS IRIWEETEDQ VFLEEEKEKE LEEQYEDTLL TSLEEGNGDD
              730 740 750 760 770 780
AFKADASGEG VEDEASGVHK QTLESLKAGE RLMEALDLGI AEIEGLEAYN RDMKLWQRKK
              790 800 810 820 830 840
LGEAPIKPQG NAVLIAVNKT PEQYIMDTLL RIRMSQLEDA LMVMPFSYVL KFLKFIDTVM
              850 860 870 880 890 900
QNKTLLHSHL PLICKNLFFI IKFNHKELVS QKNEELKLQI NRVKTELRSA LKSTEDDLGF
              910 920 930 940
NVQGLKFVKQ QWNLRHNYEF VDEYDQQEKE SNSARKRVFG TVI
SEQ ID No: 16: UTP13_Yeast (http://beta.uniprot.org/uniprot/Q05946)
                10 20 30 40 50 60
MDLKTSYKGI SLNPIYAGSS AVATVSENGK ILATPVLDEI NIIDLTPGSR KILHKISNED
                70 80 90 100 110 120
EQEITALKLT PDGQYLTYVS QAQLLKIFHL KTGKVVRSMK ISSPSYILDA DSTSTLLAVG
              130 140 150 160 170 180
GTDGSIIVVD IENGYITHSF KGHGGTISSL KFYGQLNSKI WLLASGDTNG MVKVWDLVKR
              190 200 210 220 230 240
KCLHTLQEHT SAVRGLDIIE VPDNDEPSLN LLSGGRDDII NLWDFNMKKK CKLLKTLPVN
              250 260 270 280 290 300
QQVESCGFLK DGDGKRIIYT AGGDAIFQLI DSESGSVLKR TNKPIEELFI IGVLPILSNS
              310 320 330 340 350 360
QMFLVLSDQT LQLINVEEDL KNDEDTIQVT SSIAGNHGII ADMRYVGPEL NKLALATNSP
              370 380 390 400 410 420
SLRIIPVPDL SGPEASLPLD VEIYEGHEDL LNSLDATEDG LWIATASKDN TAIVWRYNEN
              430 440 450 460 470 480
SCKFDIYAKY IGHSAAVTAV GLPNIVSKGY PEFLLTASND LTIKKWIIPK PTASMDVQII
              490 500 510 520 530 540
KVSEYTRHAH EKDINALSVS PNDSIFATAS YDKTCKIWNL ENGELEATLA NHKRGLWDVS
              550 560 570 580 590 600
FCQYDKLLAT SSGDKTVKIW SLDTFSVMKT LEGHTNAVQR CSFINKQKQL ISCGADGLIK
              610 620 630 640 650 660
IWDCSSGECL KTLDGHNNRL WALSTMNDGD MIVSADADGV FQFWKDCTEQ EIEEEQEKAK
              670 680 690 700 710 720
LQVEQEQSLQ NYMSKGDWTN AFLLAMTLDH PMRLFNVLKR ALGESRSRQD TEEGKIEVIF
              730 740 750 760 770 780
NEELDQAISI LNDEQLILLM KRCRDWNTNA KTHTIAQRTI RCILMHHNIA KLSEIPGMVK
              790 800 810
IVDAIIPYTQ RHFTRVDNLV EQSYILDYAL VEMDKLF
SEQ ID No: 17: YL409_Yeast (http://beta.uniprot.org/uniprot/Q06078)
                10 20 30 40 50 60
MSIDLKKRKV EEDVRSRGKN SKIFSPFRII GNVSNGVPFA TGTLGSTFYI VTCVGKTFQI
                70 80 90 100 110 120
YDANTLHLLF VSEKETPSSI VALSAHFHYV YAAYENKVGI YKRGIEEHLL ELETDANVEH
              130 140 150 160 170 180
LCIFGDYLCA STDDNSIFIY KKSDPQDKYP SEFYTKLTVT EIQGGEIVSL QHLATYLNKL
              190 200 210 220 230 240
TVVTKSNVLL FNVRTGKLVF TSNEFPDQIT TAEPAPVLDI IALGTVTGEV IMFNMRKGKR
              250 260 270 280 290 300
IRTIKIPQSR ISSLSFRTDG SSHLSVGTSS GDLIFYDLDR RSRIHVLKNI HRESYGGVTQ
              310 320 330 340 350 360
ATFLNGQPII VTSGGDNSLK EYVFDPSLSQ GSGDVVVQPP RYLRSRGGHS QPPSYIAFAD
              370 380 390 400 410 420
SQSHFMLSAS KDRSLWSFSL RKDAQSQEMS QRLHKKQDGG RVGGSTIKSK FPEIVALAIE
              430 440 450 460 470 480
NARIGEWENI ITAHKDEKFA RTWDMRNKRV GRWTFDTTDD GFVKSVAMSQ CGNFGFIGSS
              490 500 510 520 530 540
NGSITIYNMQ SGILRKKYKL HKRAVTGISL DGMNRKMVSC GLDGIVGFYD FNKSTLLGKL
              550 560 570 580 590 600
KLDAPITAMV YHRSSDLFAL ALDDLSIVVI DAVTQRVVRQ LWGHSNRITA FDFSPEGRWI
              610 620 630 640 650 660
VSASLDSTIR TWDLPTGGCI DGIIVDNVAT NVKFSPNGDL LATTHVTGNG ICIWTNRAQF
              670 680 690 700 710 720
KTVSTRTIDE SEFARMALPS TSVRGNDSML SGALESNGGE DLNDIDFNTY TSLEQIDKEL
              730 740 750 760 770 780
LTLSIGPRSK MNTLLHLDVI RKRSKPKEAP KKSEKLPFFL QLSGEKVGDE ASVREGIAHE
              790 800 810 820 830 840
TPEEIHRRDQ EAQKKLDAEE QMNKFKVTGR LGFESHFTKQ LREGSQSKDY SSLLATLINF
              850 860 870 880 890 900
SPAAVDLEIR SLNSFEPFDE IVWFIDALTQ GLKSNKNFEL YETFMSLLFK AHGDVIHANN
              910 920 930
KNQDIASALQ NWEDVHKKED RLDDLVKFCM GVAAFVTTA
SEQ ID No: 18: NOC4_Yeast (http://beta.uniprot.org/uniprot/Q06512)
                10 20 30 40 50 60
MVLLISEIKD IAKRLTAAGD RKQYNSIIKL INELVIPENV TQLEEDETEK NLRFLVMSLF
                70 80 90 100 110 120
QIFRKLFSRG DLTLPSSKKS TLEKEQFVNW CRKVYEAFKT KLLAIISDIP FETSLGLDSL
              130 140 150 160 170 180
DVYLQLAELE STHFASEKGA PFFPNKTFRK LIIALWSSNM GEIEDVKSSG ASENLIIVEF
              190 200 210 220 230 240
TEKYYTKFAD IQYYFQSEFN QLLEDPAYQD LLLKNVGKWL ALVNHDKHCS SVDADLEIFV
              250 260 270 280 290 300
PNPPQAIENE SKFKSNFEKN WLSLLNGQLS LQQYKSILLI LHKRIIPHFH TPTKLMDFLT
              310 320 330 340 350 360
DSYNLQSSNK NAGVVPILAL NGLFELMKRF NLEYPNFYMK LYQIINPDLM HVKYRARFFR
              370 380 390 400 410 420
LMDVFLSSTH LSAHLVASFI KKLARLTLES PPSAIVTVIP FIYNLIRKHP NCMIMLHNPA
              430 440 450 460 470 480
FISNPFQTPD QVANLKTLKE NYVDPFDVHE SDPELTHALD SSLWELASLM EHYHPNVATL
              490 500 510 520 530 540
AKIFAQPFKK LSYNMEDFLD WNYDSLLNAE SSRKLKTLPT LEFEAFTNVF DNEDGDSEAS
              550
SQGNVYLPGV AW
SEQ ID No: 19: UTP6_Yeast (http://beta.uniprot.org/uniprot/Q02354)
                10 20 30 40 50 60
MSKTRYYLEQ CIPEMDDLVE KGLFTKNEVS LIMKKRTDFE HRLNSRGSSI NDYIKYINYE
                70 80 90 100 110 120
SNVNKLRAKR CKRILQVKKT NSLSDWSIQQ RIGFIYQRGT NKFPQDLKFW AMYLNYMKAR
              130 140 150 160 170 180
GNQTSYKKIH NIYNQLLKLH PTNVDIWISC AKYEYEVHAN FKSCRNIFQN GLRFNPDVPK
              190 200 210 220 230 240
LWYEYVKFEL NFITKLINRR KVMGLINERE QELDMQNEQK NNQAPDEEKS HLQVPSTGDS
              250 260 270 280 290 300
MKDKLNELPE ADISVLGNAE TNPALRGDIA LTIFDVCMKT LGKHYINKHK GYYAISDSKM
              310 320 330 340 350 360
NIELNKETLN YLFSESLRYI KLFDEFLDLE RDYLINHVLQ FWKNDMYDLS LRKDLPELYL
              370 380 390 400 410 420
KTVMIDITLN IRYMPVEKLD IDQLQLSVKK YFAYISKLDS ASVKSLKNEY RSYLQDNYLK
              430 440
KMNAEDDPRY KILDLIISKL
```

### ANNEX I

TABLE
Total of variants found in patients and controls from 454
sequencing analysis of all exons of CYP1B1, UTP20, MPP10,
WDR46, NOC4L, WDR36, TBL3, UTP18 and PWP2 genes.

| Gene | Variant | Patients (%) | Controls (%) |
|---|---|---|---|
| CYP1B1 | 903: C/G | 50.00 | 50.00 |
| CYP1B1 | 1014: T/C | 1.20 | 0.00 |
| CYP1B1 | 1018: A/G | 0.24 | 1.04 |
| CYP1B1 | 1020: A/G | 0.48 | 1.04 |
| CYP1B1 | 1054: A/G | 0.96 | 0.26 |
| CYP1B1 | 1116: G/T | 37.35 | 30.11 |
| CYP1B1 | 1479: C/T | 0.93 | 0.00 |
| CYP1B1 | 1563: T/C | 0.95 | 0.00 |
| CYP1B1 | 1729: C/T | 1.72 | 0.00 |
| CYP1B1 | 5046: A/G | 0.70 | 0.00 |
| CYP1B1 | 5050: T/C | 0.56 | 0.00 |
| CYP1B1 | 5074: T/C | 0.85 | 0.00 |
| CYP1B1 | 5082: A/G | 0.71 | 0.00 |
| CYP1B1 | 5090: G/C | 39.19 | 33.93 |
| CYP1B1 | 5094: A/G | 0.72 | 0.00 |
| CYP1B1 | 5106: C/T | 2.48 | 0.00 |
| CYP1B1 | 5115: T/C | 0.59 | 0.00 |
| CYP1B1 | 5119: T/C | 0.59 | 0.00 |
| CYP1B1 | 5126: C/T | 0.90 | 0.00 |
| CYP1B1 | 5143: T/C | 42.90 | 50.00 |
| CYP1B1 | 5143: T/G | 0.65 | 0.00 |
| CYP1B1 | 5144: G/C | 0.82 | 0.00 |
| CYP1B1 | 5154: A/G | 5.12 | 6.06 |
| CYP1B1 | 5171: A/G | 0.69 | 0.00 |
| CYP1B1 | 5341: A/G | 2.47 | 0.00 |
| CYP1B1 | 5401: T/C | 1.11 | 0.00 |
| MPP10 | 2551: G/A | 11.73 | 12.75 |
| MPP10 | 2555: T/C | 0.56 | 0.00 |
| MPP10 | 2567: C/T | 12.85 | 14.74 |
| MPP10 | 2582: T/C | 0.70 | 0.00 |
| MPP10 | 2587: T/C | 0.56 | 0.00 |
| MPP10 | 2594: G/T | 12.75 | 13.25 |
| MPP10 | 2601: G/T | 12.85 | 13.82 |
| MPP10 | 2617: C/A | 1.71 | 1.23 |
| MPP10 | 2622: A/G | 0.00 | 3.29 |
| MPP10 | 2625: A/G | 0.57 | 0.00 |
| MPP10 | 2631: C/G | 13.06 | 13.17 |
| MPP10 | 2657: G/A | 1.76 | 1.27 |
| MPP10 | 2663: T/A | 13.50 | 15.22 |
| MPP10 | 2670: C/T | 13.06 | 15.62 |
| MPP10 | 2671: A/G | 13.25 | 15.62 |
| MPP10 | 2681: C/T | 13.35 | 14.93 |
| MPP10 | 2684: G/A | 13.37 | 15.38 |
| MPP10 | 2701: A/C | 24.35 | 11.52 |
| MPP10 | 2703: A/T | 1.23 | 0.46 |
| MPP10 | 2704: A/G | 13.71 | 15.74 |
| MPP10 | 2704: A/T | 4.01 | 1.39 |
| MPP10 | 2705: T/A | 0.78 | 0.47 |
| MPP10 | 2706: T/A | 2.19 | 0.93 |
| MPP10 | 2707: T/A | 1.69 | 0.48 |
| MPP10 | 2708: T/G | 1.71 | 0.49 |
| MPP10 | 2709: G/T | 1.76 | 0.49 |
| MPP10 | 2722: T/C | 15.68 | 14.43 |
| MPP10 | 2725: G/T | 15.50 | 14.50 |
| MPP10 | 2764: A/G | 16.07 | 16.32 |
| MPP10 | 2768: T/C | 15.59 | 15.79 |
| MPP10 | 2771: G/A | 15.46 | 16.04 |
| MPP10 | 2772: A/G | 0.77 | 0.00 |
| MPP10 | 2794-2795: AT/— | 18.65 | 12.20 |
| MPP10 | 2811: C/A | 5.57 | 3.63 |
| MPP10 | 2812-2814: TTC/— | 5.57 | 3.63 |
| MPP10 | 2837: A/G | 5.76 | 3.63 |
| MPP10 | 2839: G/A | 20.89 | 35.91 |
| MPP10 | 2860: T/C | 5.80 | 4.04 |
| MPP10 | 2872: A/G | 5.80 | 3.70 |
| MPP10 | 2874: A/C | 5.80 | 3.72 |
| MPP10 | 2912: C/A | 5.84 | 3.74 |
| MPP10 | 2915: C/T | 5.87 | 3.74 |
| MPP10 | 2922: A/G | 5.87 | 3.74 |
| MPP10 | 2923: A/G | 5.87 | 3.74 |
| MPP10 | 2927: T/C | 6.07 | 3.74 |
| MPP10 | 2928: G/A | 5.87 | 3.74 |
| MPP10 | 2937: A/G | 6.07 | 4.08 |
| MPP10 | 2940: G/A | 5.68 | 4.08 |
| MPP10 | 2951-2953: TGA/— | 5.70 | 3.41 |
| MPP10 | 2956: A/G | 2.75 | 2.73 |
| MPP10 | 2968: C/T | 5.74 | 3.75 |
| MPP10 | 2969: T/C | 5.56 | 3.75 |
| MPP10 | 2974: T/C | 5.56 | 4.10 |
| MPP10 | 2977: G/C | 4.97 | 3.75 |
| MPP10 | 2987: C/A | 5.79 | 3.75 |
| MPP10 | 2988: G/A | 0.00 | 1.71 |
| MPP10 | 2991: T/C | 5.80 | 3.77 |
| MPP10 | 3028: G/A | 20.53 | 13.89 |
| MPP10 | 3055: A/G | 17.41 | 12.15 |
| MPP10 | 3068: G/C | 19.84 | 15.09 |
| MPP10 | 3071: A/G | 19.84 | 15.09 |
| MPP10 | 3074: A/G | 1.32 | 0.00 |
| MPP10 | 3094: T/C | 20.00 | 16.04 |
| MPP10 | 3098: T/C | 11.73 | 12.26 |
| MPP10 | 3099: G/A | 1.33 | 0.00 |
| MPP10 | 3101: T/G | 20.00 | 15.09 |
| MPP10 | 3111: A/C | 20.00 | 15.24 |
| MPP10 | 3144-3145: AT/— | 1.34 | 0.00 |
| MPP10 | 3145-3146: TA/— | 18.28 | 15.38 |
| MPP10 | 3146: A/G | 1.34 | 0.00 |
| MPP10 | 3150: A/G | 2.96 | 0.00 |
| MPP10 | 3172: A/G | 19.68 | 15.38 |
| MPP10 | 3176: T/C | 1.62 | 1.92 |
| MPP10 | 3182: G/T | 45.82 | 39.42 |
| MPP10 | 3185: G/C | 19.68 | 15.38 |
| MPP10 | 3187: A/T | 19.78 | 15.38 |
| MPP10 | 3188: G/A | 19.84 | 15.38 |
| MPP10 | 3196: T/C | 20.00 | 15.38 |
| MPP10 | 3214: T/C | 3.63 | 7.69 |
| MPP10 | 3238: G/C | 20.34 | 15.38 |
| MPP10 | 3246: G/A | 20.40 | 15.38 |
| MPP10 | 3269: G/A | 18.90 | 15.53 |
| MPP10 | 10978: G/A | 0.60 | 0.00 |
| MPP10 | 14106: T/C | 0.95 | 0.00 |
| MPP10 | 14134: A/G | 0.96 | 0.00 |
| MPP10 | 14275: A/G | 1.74 | 0.00 |
| MPP10 | 14299: C/A | 2.50 | 0.00 |
| MPP10 | 17676: T/C | 1.80 | 0.00 |
| MPP10 | 18931: A/G | 0.80 | 0.00 |
| MPP10 | 18949: A/G | 0.80 | 0.00 |
| MPP10 | 18985: A/G | 1.01 | 0.00 |
| MPP10 | 19058: A/G | 0.58 | 0.00 |
| MPP10 | 19104: A/G | 0.58 | 0.00 |
| MPP10 | 19556: G/A | 31.77 | 16.51 |
| MPP10 | 19600: T/C | 1.47 | 0.00 |
| NOC4L | 94: T/C | 0.17 | 0.66 |
| NOC4L | 111: A/G | 0.00 | 0.67 |
| NOC4L | 129: G/A | 0.00 | 1.51 |
| NOC4L | 160: T/C | 0.00 | 0.67 |
| NOC4L | 171: G/A | 0.00 | 0.67 |
| NOC4L | 211: A/G | 0.17 | 0.68 |
| NOC4L | 414: A/G | 0.50 | 0.21 |
| NOC4L | 417: A/G | 0.50 | 0.21 |
| NOC4L | 446: T/C | 0.51 | 0.00 |
| NOC4L | 453: G/A | 0.13 | 3.57 |
| NOC4L | 456: T/C | 0.77 | 0.00 |
| NOC4L | 481: A/G | 0.39 | 0.86 |
| NOC4L | 497: C/T | 0.52 | 0.00 |
| NOC4L | 508: T/C | 0.66 | 0.00 |
| NOC4L | 516: T/C | 0.53 | 0.00 |
| NOC4L | 524: A/G | 0.80 | 0.00 |
| NOC4L | 537: G/A | 0.53 | 0.00 |
| NOC4L | 546: C/G | 0.54 | 0.44 |
| NOC4L | 550: G/C | 1.75 | 2.84 |
| NOC4L | 2828: T/C | 1.45 | 0.00 |
| NOC4L | 2863: C/T | 5.07 | 14.43 |
| NOC4L | 2870: C/T | 0.00 | 1.99 |
| NOC4L | 2895: A/G | 1.23 | 0.00 |
| NOC4L | 2953-2975: DEL(23) | 0.00 | 6.53 |
| NOC4L | 3685: A/T | 3.07 | 0.00 |
| NOC4L | 3686: C/A | 3.07 | 0.00 |
| NOC4L | 3687-3689: CCT/— | 81.76 | 79.07 |
| NOC4L | 3688-3689: CT/— | 84.91 | 79.07 |
| NOC4L | 4096: T/C | 1.00 | 0.00 |
| NOC4L | 4107: C/T | 0.25 | 4.31 |
| NOC4L | 6529: T/C | 90.82 | 74.64 |
| NOC4L | 6604: A/G | 0.85 | 0.00 |
| NOC4L | 6723: A/G | 0.55 | 0.00 |
| NOC4L | 6741: A/G | 0.68 | 0.00 |
| NOC4L | 6752: T/C | 0.55 | 0.00 |
| NOC4L | 6776: T/C | 0.56 | 0.00 |
| NOC4L | 6782: A/G | 0.56 | 0.00 |
| NOC4L | 6783: T/C | 0.83 | 0.00 |
| NOC4L | 6787: C/T | 52.73 | 75.68 |
| NOC4L | 6788: T/C | 0.56 | 0.00 |
| NOC4L | 6791: T/C | 0.59 | 0.00 |
| NOC4L | 6820: T/C | 0.74 | 0.00 |
| NOC4L | 6859: T/C | 0.90 | 0.00 |
| NOC4L | 6891: T/C | 0.91 | 0.00 |
| NOC4L | 6905: C/T | 0.61 | 0.00 |
| NOC4L | 6912: T/C | 0.76 | 0.00 |
| NOC4L | 6925: A/G | 0.61 | 1.47 |
| NOC4L | 6934: G/A | 0.61 | 0.00 |
| NOC4L | 6939: T/C | 1.54 | 1.47 |
| NOC4L | 6950: T/C | 0.62 | 0.00 |
| NOC4L | 6963: T/C | 10.47 | 0.00 |
| NOC4L | 6968: C/T | 5.26 | 0.00 |
| NOC4L | 6970: T/C | 5.48 | 0.00 |
| NOC4L | 7408: G/A | 19.76 | 25.94 |
| NOC4L | 7437: A/G | 0.79 | 0.32 |
| NOC4L | 7450: A/G | 0.23 | 1.92 |
| NOC4L | 7587: A/G | 1.27 | 0.00 |
| NOC4L | 7703: A/G | 1.31 | 0.00 |
| PWP2 | 84: G/A | 27.38 | 22.11 |
| PWP2 | 208: T/C | 1.08 | 0.72 |
| PWP2 | 253: G/A | 76.42 | 84.42 |
| PWP2 | 272: G/C | 1.09 | 0.73 |
| PWP2 | 273: C/G | 1.09 | 0.73 |
| PWP2 | 276: T/C | 1.37 | 0.37 |
| PWP2 | 1712: G/A | 72.93 | 70.59 |
| PWP2 | 1809: A/G | 2.20 | 0.00 |
| PWP2 | 6519: A/G | 1.28 | 0.00 |
| PWP2 | 6825: C/T | 58.75 | 68.32 |
| PWP2 | 7022: C/T | 2.46 | 0.00 |
| PWP2 | 8445: T/A | 2.36 | 0.79 |
| PWP2 | 8457: A/G | 2.04 | 0.79 |
| PWP2 | 8499: G/C | 2.09 | 1.61 |
| PWP2 | 8501: A/G | 1.74 | 4.03 |
| PWP2 | 8503: C/G | 2.12 | 0.00 |
| PWP2 | 10596: T/C | 1.20 | 0.00 |
| PWP2 | 10641: A/G | 1.51 | 0.00 |
| PWP2 | 11361: T/C | 0.00 | 4.88 |
| PWP2 | 11440: T/C | 62.07 | 68.64 |
| PWP2 | 11518: C/A | 66.93 | 72.22 |
| PWP2 | 12033: G/C | 64.27 | 73.71 |
| PWP2 | 12034-12036: GTA/— | 1.19 | 0.58 |
| PWP2 | 12034: G/T | 0.00 | 4.09 |
| PWP2 | 12219: G/A | 67.81 | 75.46 |
| PWP2 | 13000: C/T | 67.64 | 80.20 |
| PWP2 | 13024: C/G | 0.00 | 2.48 |
| PWP2 | 13049: T/C | 0.78 | 0.00 |
| PWP2 | 13056: T/C | 0.78 | 0.00 |
| PWP2 | 13071: A/G | 0.31 | 1.98 |
| PWP2 | 13081: C/T | 0.16 | 7.00 |
| PWP2 | 13153: A/G | 0.63 | 0.00 |
| PWP2 | 13169: A/G | 0.64 | 0.00 |
| PWP2 | 13195: T/C | 0.65 | 0.00 |
| PWP2 | 13332: T/C | 0.43 | 4.58 |
| PWP2 | 13405: T/C | 1.83 | 0.00 |
| PWP2 | 13498: T/C | 2.90 | 0.00 |
| PWP2 | 13712: A/G | 59.76 | 79.13 |
| PWP2 | 14865: T/A | 3.68 | 0.00 |
| PWP2 | 17387: T/C | 79.79 | 83.54 |
| PWP2 | 18686: A/G | 2.55 | 0.00 |
| PWP2 | 19613: T/C | 0.29 | 4.72 |
| PWP2 | 19716: A/G | 1.17 | 0.00 |
| PWP2 | 20706: C/A | 12.44 | 0.00 |
| PWP2 | 20719: G/A | 0.00 | 10.64 |
| PWP2 | 20744: C/T | 0.00 | 10.64 |
| PWP2 | 21023: T/C | 0.56 | 0.00 |
| PWP2 | 21025: T/C | 0.85 | 0.00 |
| PWP2 | 21208: A/G | 1.17 | 0.00 |
| PWP2 | 21288: A/G | 2.07 | 0.00 |
| TBL3 | 1977: T/C | 0.63 | 0.33 |
| TBL3 | 2179: A/G | 1.86 | 0.00 |
| TBL3 | 2292: G/T | 11.33 | 15.56 |
| TBL3 | 3109: G/A | 0.70 | 0.00 |
| TBL3 | 3134: T/C | 0.70 | 0.00 |
| TBL3 | 3143: A/C | 24.82 | 18.92 |
| TBL3 | 3180: A/G | 1.08 | 0.55 |
| TBL3 | 3348: G/C | 4.99 | 9.64 |
| TBL3 | 3895: G/A | 6.84 | 0.00 |
| TBL3 | 4001: C/A | 6.08 | 2.45 |
| TBL3 | 4150: A/G | 1.63 | 0.00 |
| TBL3 | 4201: T/C | 1.10 | 0.83 |
| TBL3 | 4229: T/C | 11.05 | 18.33 |
| TBL3 | 4315: G/A | 11.27 | 18.58 |
| TBL3 | 4828: T/C | 9.09 | 9.26 |
| TBL3 | 5055: A/G | 2.05 | 0.00 |
| TBL3 | 5460: A/G | 1.85 | 0.00 |
| TBL3 | 5703: A/G | 0.74 | 0.67 |
| TBL3 | 5859: C/T | 0.00 | 1.83 |
| TBL3 | 5892: A/G | 8.28 | 22.94 |
| TBL3 | 6010: A/G | 2.45 | 15.16 |
| TBL3 | 6011: C/A | 2.46 | 15.16 |
| TBL3 | 6260: T/C | 1.04 | 0.00 |
| TBL3 | 6339: A/C | 16.62 | 16.67 |
| TBL3 | 6444: G/A | 0.27 | 3.75 |
| TBL3 | 6579: G/A | 0.00 | 1.69 |
| TBL3 | 6614: A/G | 20.00 | 4.84 |
| UTP18 | 73: A/G | 2.86 | 0.00 |
| UTP18 | 5614: A/G | 1.73 | 0.00 |
| UTP18 | 5762: A/G | 10.94 | 8.80 |
| UTP18 | 8216: A/G | 3.29 | 0.00 |
| UTP18 | 12837: A/G | 1.35 | 0.00 |
| UTP18 | 12906: A/C | 58.70 | 45.65 |
| UTP18 | 16602: G/A | 0.62 | 0.00 |
| UTP18 | 16656: A/G | 0.63 | 0.00 |
| UTP18 | 16681: A/G | 0.63 | 3.03 |
| UTP18 | 16755: T/C | 0.64 | 0.00 |
| UTP18 | 24708: A/G | 2.78 | 0.00 |
| UTP18 | 33334: G/C | 61.00 | 54.35 |
| UTP18 | 33428: T/C | 0.00 | 4.55 |
| UTP18 | 36353: T/C | 0.00 | 5.38 |
| UTP18 | 36464: T/C | 0.00 | 4.49 |
| UTP20 E1-31 | 140: A/G | 0.98 | 0.00 |
| UTP20 E1-31 | 196: T/C | 1.73 | 0.00 |
| UTP20 E1-31 | 237: T/C | 1.49 | 0.67 |
| UTP20 E1-31 | 285: T/C | 1.51 | 0.00 |
| UTP20 E1-31 | 305: T/C | 1.01 | 0.00 |
| UTP20 E1-31 | 318: G/A | 3.41 | 0.00 |
| UTP20 E1-31 | 5593: A/G | 37.92 | 0.00 |
| UTP20 E1-31 | 5594: A/G | 12.18 | 0.00 |
| UTP20 E1-31 | 5595: A/C | 2.99 | 0.00 |
| UTP20 E1-31 | 5730: A/G | 1.15 | 0.00 |
| UTP20 E1-31 | 5754: G/A | 6.96 | 3.70 |
| UTP20 E1-31 | 5818: T/C | 1.79 | 0.00 |
| UTP20 E1-31 | 5831: T/C | 1.51 | 0.00 |
| UTP20 E1-31 | 10025: C/T | 53.51 | 48.72 |
| UTP20 E1-31 | 10156: A/C | 3.02 | 0.00 |
| UTP20 E1-31 | 10591: A/G | 2.52 | 0.00 |
| UTP20 E1-31 | 10593: T/C | 1.26 | 5.00 |
| UTP20 E1-31 | 11787: G/A | 16.43 | 17.14 |
| UTP20 E1-31 | 11948: G/A | 12.06 | 16.84 |
| UTP20 E1-31 | 11978: A/G | 2.64 | 0.00 |
| UTP20 E1-31 | 15401: T/C | 1.63 | 0.00 |
| UTP20 E1-31 | 15475: C/T | 2.13 | 0.00 |
| UTP20 E1-31 | 19576: A/G | 1.93 | 0.00 |
| UTP20 E1-31 | 19630: C/G | 22.89 | 31.96 |
| UTP20 E1-31 | 19657: A/G | 2.49 | 0.00 |
| UTP20 E1-31 | 19780-19781: TG/— | 2.17 | 0.00 |
| UTP20 E1-31 | 19988: G/A | 13.57 | 15.83 |
| UTP20 E1-31 | 19993: T/A | 1.55 | 0.00 |
| UTP20 E1-31 | 22383: A/G | 2.12 | 0.00 |
| UTP20 E1-31 | 26576: C/T | 1.72 | 0.00 |
| UTP20 E1-31 | 28144: A/G | 0.88 | 0.00 |
| UTP20 E1-31 | 29642: G/A | 2.84 | 4.00 |
| UTP20 E1-31 | 31529: T/C | 32.32 | 44.20 |
| UTP20 E1-31 | 31558: A/G | 0.00 | 2.23 |
| UTP20 E1-31 | 31573: T/C | 15.09 | 19.55 |
| UTP20 E1-31 | 31585: T/C | 3.97 | 1.12 |
| UTP20 E1-31 | 31588: C/T | 3.80 | 1.12 |
| UTP20 E1-31 | 31591-31592: AG/— | 1.15 | 1.12 |
| UTP20 E1-31 | 31649: T/C | 0.20 | 2.26 |
| UTP20 E1-31 | 37496: T/C | 2.49 | 0.00 |
| UTP20 E1-31 | 37526: A/G | 3.32 | 0.00 |
| UTP20 E1-31 | 47012: T/C | 2.79 | 0.00 |
| UTP20 E1-31 | 49135: A/G | 1.92 | 0.00 |
| UTP20 E1-31 | 49204: A/G | 2.02 | 0.00 |
| UTP20 E1-31 | 53349: A/G | 2.66 | 0.00 |
| UTP20 E1-31 | 54262: A/G | 22.41 | 20.63 |
| UTP20 E1-31 | 58022: G/A | 2.02 | 0.00 |
| UTP20 E1-31 | 58129: T/C | 0.59 | 0.00 |
| UTP20 E1-31 | 58214: G/A | 0.59 | 0.00 |
| UTP20 E1-31 | 58246: G/A | 1.63 | 0.00 |
| UTP20 E1-31 | 58751: C/A | 9.09 | 1.20 |
| UTP20 E1-31 | 58751: C/T | 20.25 | 22.89 |
| UTP20 E1-31 | 58753: A/C | 9.13 | 1.20 |
| UTP20 E1-31 | 58759: C/A | 3.27 | 0.00 |
| UTP20 E1-31 | 58760: A/C | 3.27 | 0.00 |
| UTP20 E32-62 | 996: T/C | 1.49 | 0.00 |
| UTP20 E32-62 | 1026: T/C | 19.78 | 28.95 |
| UTP20 E32-62 | 1107: T/C | 1.50 | 0.00 |
| UTP20 E32-62 | 3130: A/G | 23.06 | 18.59 |
| UTP20 E32-62 | 3130: A/T | 4.09 | 3.52 |
| UTP20 E32-62 | 3132: T/A | 4.09 | 2.01 |
| UTP20 E32-62 | 3133: T/A | 0.86 | 1.01 |
| UTP20 E32-62 | 3136-3137: AC/— | 1.09 | 0.00 |
| UTP20 E32-62 | 3136: A/T | 0.87 | 1.01 |
| UTP20 E32-62 | 3145: C/T | 0.92 | 0.00 |
| UTP20 E32-62 | 3160: A/T | 0.93 | 0.54 |
| UTP20 E32-62 | 3161: T/A | 0.93 | 0.55 |
| UTP20 E32-62 | 3261: A/G | 1.46 | 0.00 |
| UTP20 E32-62 | 3264: A/G | 1.46 | 0.00 |
| UTP20 E32-62 | 3317: C/T | 0.66 | 0.76 |
| UTP20 E32-62 | 3356: G/A | 1.54 | 0.00 |
| UTP20 E32-62 | 3595: T/C | 0.00 | 4.82 |
| UTP20 E32-62 | 5155: A/G | 8.80 | 0.00 |
| UTP20 E32-62 | 5214: T/C | 8.84 | 0.00 |
| UTP20 E32-62 | 5261-5263: TGA/— | 0.47 | 4.60 |
| UTP20 E32-62 | 6938: G/A | 1.40 | 0.00 |
| UTP20 E32-62 | 6982: A/G | 1.13 | 0.00 |
| UTP20 E32-62 | 12696: C/T | 0.74 | 0.00 |
| UTP20 E32-62 | 13772: T/C | 3.21 | 0.00 |
| UTP20 E32-62 | 15384: A/G | 1.11 | 0.00 |
| UTP20 E32-62 | 15539: C/T | 1.73 | 0.00 |
| UTP20 E32-62 | 17054: T/C | 1.21 | 0.00 |
| UTP20 E32-62 | 17206: A/G | 1.02 | 0.00 |
| UTP20 E32-62 | 17270: T/C | 1.03 | 0.00 |
| UTP20 E32-62 | 17563: T/A | 98.17 | 97.50 |
| UTP20 E32-62 | 22432: T/C | 29.44 | 11.11 |
| UTP20 E32-62 | 24279: G/A | 0 | 15.79 |
| UTP20 E32-62 | 27065: T/C | 1.64 | 1.20 |
| UTP20 E32-62 | 27102: C/T | 1.65 | 0.00 |
| UTP20 E32-62 | 27166: A/G | 1.65 | 0.00 |
| UTP20 E32-62 | 27201: A/G | 2.11 | 0.00 |
| UTP20 E32-62 | 28507: A/G | 0.00 | 3.85 |
| UTP20 E32-62 | 30252: T/C | 3.25 | 0.00 |
| UTP20 E32-62 | 30432-30433: AC/— | 30.00 | 66.67 |
| UTP20 E32-62 | 30899: T/C | 2.12 | 0.00 |
| UTP20 E32-62 | 30981: A/G | 1.78 | 0.00 |
| UTP20 E32-62 | 31009: A/G | 2.22 | 0.00 |
| UTP20 E32-62 | 31011: A/G | 1.78 | 0.00 |
| UTP20 E32-62 | 31061: A/G | 1.82 | 0.00 |
| UTP20 E32-62 | 33282: T/C | 1.00 | 0.00 |
| UTP20 E32-62 | 33977: C/T | 0.77 | 0.00 |
| UTP20 E32-62 | 40031: A/C | 1.67 | 1.19 |
| UTP20 E32-62 | 40032: C/A | 2.08 | 1.19 |
| UTP20 E32-62 | 41746: T/C | 0.00 | 2.14 |
| UTP20 E32-62 | 43653: A/G | 0.12 | 1.84 |
| UTP20 E32-62 | 43681: T/C | 0.60 | 0.00 |
| UTP20 E32-62 | 43708: G/A | 0.24 | 3.38 |
| UTP20 E32-62 | 43745: G/C | 11.17 | 8.90 |
| UTP20 E32-62 | 43859: A/G | 1.91 | 0.71 |
| UTP20 E32-62 | 43862: A/G | 1.10 | 0.00 |
| UTP20 E32-62 | 44038: T/C | 1.48 | 0.00 |
| UTP20 E32-62 | 46125: A/G | 0.59 | 0.00 |
| UTP20 E32-62 | 46130: T/C | 1.18 | 0.00 |
| UTP20 E32-62 | 46137: A/G | 0.59 | 0.00 |
| UTP20 E32-62 | 46146: A/G | 0.59 | 0.00 |
| UTP20 E32-62 | 46157: A/G | 0.89 | 0.00 |
| UTP20 E32-62 | 46158: A/G | 0.60 | 0.39 |
| UTP20 E32-62 | 46225: G/A | 0.62 | 1.20 |
| UTP20 E32-62 | 46244: A/G | 0.78 | 0.00 |
| UTP20 E32-62 | 46251: T/C | 0.63 | 0.40 |
| UTP20 E32-62 | 46254: A/G | 0.63 | 0.00 |
| UTP20 E32-62 | 46273: A/G | 0.80 | 0.00 |
| UTP20 E32-62 | 46713: T/C | 88.28 | 87.04 |
| UTP20 E32-62 | 46753: T/G | 1.85 | 0.00 |
| UTP20 E32-62 | 46754: T/G | 6.81 | 12.77 |
| UTP20 E32-62 | 46755: G/T | 9.49 | 13.04 |
| WDR3 | 4841: T/C | 1.55 | 0.00 |
| WDR3 | 8677: A/G | 0.60 | 0.40 |
| WDR3 | 8679: A/G | 0.00 | 1.59 |
| WDR3 | 8808: G/A | 0.62 | 0.00 |
| WDR3 | 8814: A/G | 0.15 | 2.05 |
| WDR3 | 9759: C/T | 4.23 | 2.70 |
| WDR3 | 11361: T/C | 0.83 | 0.00 |
| WDR3 | 11406: A/G | 0.60 | 0.00 |
| WDR3 | 11462: A/G | 0.61 | 0.00 |
| WDR3 | 16410: A/G | 1.23 | 0.00 |
| WDR3 | 18720-18721: TG/— | 92.86 | 83.33 |
| WDR3 | 20026: T/C | 3.39 | 1.03 |
| WDR3 | 20197: T/G | 2.88 | 0.00 |
| WDR3 | 21065: A/G | 1.63 | 0.00 |
| WDR3 | 22252: A/G | 1.01 | 0.00 |
| WDR3 | 22300: A/G | 1.29 | 0.00 |
| WDR3 | 22449: G/C | 1.53 | 0.00 |
| WDR3 | 22570: T/C | 0.00 | 4.10 |
| WDR3 | 22759: A/G | 0.66 | 0.00 |
| WDR3 | 23173: T/C | 0.74 | 0.54 |
| WDR3 | 24317: A/G | 0.80 | 1.16 |
| WDR3 | 24361: A/G | 0.83 | 0.00 |
| WDR3 | 27313: A/G | 1.37 | 0.00 |
| WDR3 | 29137: T/C | 2.25 | 0.00 |
| WDR3 | 29193: A/G | 0.29 | 4.32 |
| WDR3 | 29305: T/A | 1.90 | 0.00 |
| WDR3 | 29310: T/A | 1.90 | 0.00 |
| WDR3 | 29664: T/C | 1.27 | 0.00 |
| WDR3 | 29697: C/T | 1.62 | 0.00 |
| WDR3 | 29698: T/C | 1.62 | 0.00 |
| WDR3 | 29706-29708: TTT/— | 2.92 | 0.91 |
| WDR3 | 29710: A/T | 1.95 | 1.82 |
| WDR3 | 29718: C/A | 1.96 | 1.83 |
| WDR3 | 29720: T/G | 1.31 | 1.87 |
| WDR3 | 29722: T/C | 1.97 | 0.00 |
| WDR3 | 29726: G/C | 2.12 | 2.94 |
| WDR3 | 29727: G/T | 1.77 | 1.96 |
| WDR36 | 181: T/C | 1.18 | 0.00 |
| WDR36 | 191: T/C | 5.93 | 1.25 |
| WDR36 | 215: A/G | 0.82 | 0.00 |
| WDR36 | 269: C/T | 3.83 | 0.00 |
| WDR36 | 295: G/A | 1.40 | 0.00 |
| WDR36 | 446: A/G | 4.14 | 0.00 |
| WDR36 | 2619: C/G | 39.73 | 27.66 |
| WDR36 | 2826: A/T | 13.49 | 22.73 |
| WDR36 | 2827: T/A | 13.49 | 22.73 |
| WDR36 | 6579: C/T | 3.35 | 0.00 |
| WDR36 | 6589: T/C | 1.49 | 0.00 |
| WDR36 | 6602: T/C | 1.87 | 0.00 |
| WDR36 | 8431: A/G | 0.95 | 0.62 |
| WDR36 | 8476: A/C | 1.93 | 0.00 |
| WDR36 | 8477: T/C | 1.93 | 0.00 |
| WDR36 | 8577: A/G | 0.00 | 8.00 |
| WDR36 | 8581: C/T | 31.99 | 44.00 |
| WDR36 | 10144: A/T | 5.71 | 3.26 |
| WDR36 | 10145: T/A | 6.22 | 3.26 |
| WDR36 | 11508: A/G | 1.40 | 0.00 |
| WDR36 | 11640: A/G | 12.61 | 20.00 |
| WDR36 | 11645: T/C | 1.15 | 0.00 |
| WDR36 | 13063: T/C | 1.31 | 2.16 |
| WDR36 | 14026: T/C | 0.24 | 2.79 |
| WDR36 | 14084: T/C | 1.23 | 0.57 |
| WDR36 | 15046: A/T | 6.09 | 2.38 |
| WDR36 | 15116: C/T | 0.00 | 5.00 |
| WDR36 | 15128: A/G | 2.12 | 0.00 |
| WDR36 | 15181: C/T | 0.00 | 5.06 |
| WDR36 | 15202: A/G | 2.21 | 1.27 |
| WDR36 | 15238: A/G | 2.22 | 1.32 |
| WDR36 | 17971: A/G | 8.62 | 1.03 |
| WDR36 | 17971: A/T | 3.66 | 2.06 |
| WDR36 | 17980: G/A | 3.86 | 0.00 |
| WDR36 | 18083: T/C | 1.57 | 0.00 |
| WDR36 | 18114: A/G | 1.06 | 0.00 |
| WDR36 | 18177: C/A | 1.60 | 0.00 |
| WDR36 | 18178: A/C | 1.60 | 0.00 |
| WDR36 | 18569: A/G | 0.80 | 0.00 |
| WDR36 | 18740: A/G | 1.04 | 0.00 |
| WDR36 | 18984: G/A | 1.39 | 0.00 |
| WDR36 | 19034: A/G | 1.40 | 0.00 |
| WDR36 | 19071: G/A | 1.40 | 0.00 |
| WDR36 | 20785: T/C | 3.93 | 0.00 |
| WDR36 | 20865: A/T | 6.21 | 0.00 |
| WDR36 | 20867-20868: TA/— | 6.78 | 10.00 |
| WDR36 | 20882: A/T | 2.86 | 2.00 |
| WDR36 | 20884: T/A | 7.43 | 0.00 |
| WDR36 | 20886: T/A | 3.43 | 0.00 |
| WDR36 | 20888: T/A | 2.86 | 0.00 |
| WDR36 | 20890: T/A | 4.00 | 0.00 |
| WDR36 | 20894: T/A | 18.97 | 4.00 |
| WDR36 | 20896: A/T | 22.99 | 4.00 |
| WDR36 | 20897: T/A | 0.00 | 8.00 |
| WDR36 | 20904-20905: GC/— | 4.91 | 6.38 |
| WDR36 | 26729: A/G | 0.00 | 4.21 |
| WDR36 | 26812: A/G | 14.73 | 18.09 |
| WDR36 | 28394: C/G | 7.02 | 12.50 |
| WDR36 | 28731: A/G | 0.16 | 4.55 |
| WDR36 | 28763: A/G | 0.80 | 0.00 |
| WDR36 | 28835: A/T | 16.04 | 23.58 |
| WDR36 | 28871: A/G | 0.71 | 0.00 |
| WDR36 | 28897: A/G | 1.77 | 2.06 |
| WDR36 | 28913: T/C | 1.42 | 0.00 |
| WDR36 | 31732: A/G | 0.00 | 3.68 |
| WDR36 | 32078: G/C | 20.57 | 14.75 |
| WDR36 | 33395: A/G | 2.44 | 0.00 |
| WDR36 | 33429: T/G | 1.75 | 0.00 |
| WDR36 | 33523: T/C | 1.43 | 0.00 |
| WDR36 | 33548: T/C | 1.43 | 0.00 |
| WDR36 | 34757: C/T | 2.58 | 0.00 |
| WDR46 | 303: A/G | 0.17 | 1.98 |
| WDR46 | 334: C/T | 0.68 | 0.00 |
| WDR46 | 338: T/C | 1.70 | 0.00 |
| WDR46 | 339: C/T | 1.53 | 4.15 |
| WDR46 | 521: A/G | 92.55 | 97.24 |
| WDR46 | 528: A/G | 0.87 | 0.31 |
| WDR46 | 562: A/G | 1.05 | 0.00 |
| WDR46 | 594: C/T | 0.72 | 0.94 |
| WDR46 | 595: C/T | 11.29 | 17.30 |
| WDR46 | 596: C/A | 0.75 | 0.34 |
| WDR46 | 600: A/G | 0.76 | 0.35 |
| WDR46 | 681: C/G | 2.42 | 0.00 |
| WDR46 | 1623: G/A | 12.12 | 11.11 |
| WDR46 | 1712: T/C | 0.72 | 0.00 |
| WDR46 | 1713: T/C | 0.89 | 0.00 |
| WDR46 | 1763: T/C | 2.36 | 0.00 |
| WDR46 | 1775: A/G | 0.73 | 0.00 |
| WDR46 | 1790: A/G | 0.73 | 0.51 |
| WDR46 | 1847: T/C | 0.74 | 0.00 |
| WDR46 | 2315: T/C | 1.23 | 0.72 |
| WDR46 | 2327: T/C | 11.36 | 14.49 |
| WDR46 | 8431: C/T | 0.00 | 3.37 |
| WDR46 | 8549: G/C | 1.62 | 0.00 |
| WDR46 | 8550: G/C | 1.31 | 0.00 |
| WDR46 | 8553: A/G | 1.31 | 0.00 |
| WDR46 | 8555: G/A | 1.31 | 0.00 |
| WDR46 | 8796: C/G | 9.24 | 19.63 |
| WDR46 | 9397: C/T | 3.31 | 0.00 |
| WDR46 | 9411-9412: TC/— | 3.33 | 0.00 |
| WDR46 | 9418: G/C | 3.33 | 0.00 |
| WDR46 | 9688: G/A | 0.91 | 0.00 |
| WDR46 | 9689: A/G | 0.91 | 0.00 |
| WDR46 | 9813: C/T | 0.81 | 0.00 |
| WDR46 | 9827: T/C | 1.22 | 0.00 |
| WDR46 | 9844: A/G | 0.81 | 0.57 |
| WDR46 | 9863: A/G | 0.82 | 0.57 |
| WDR46 | 9908: G/A | 0.83 | 0.00 |
| WDR46 | 9930: T/C | 1.05 | 0.00 |
| WDR46 | 9947: T/C | 3.82 | 0.00 |
| WDR46 | 9981: T/C | 0.88 | 0.00 |

1. An isolated protein complex comprising polypeptide components: (i) UTP20_HUMAN or a fragment, variant or homologue thereof; (ii) PWP2_HUMAN or a fragment, variant or homologue thereof; (iii) WDR46_HUMAN or a fragment, variant or homologue thereof; (iv) UTP18_HUMAN or a fragment, variant or homologue thereof; (v) MPP10_HUMAN or a fragment, variant or homologue thereof; (vi) WDR3_HUMAN or a fragment, variant or homologue thereof; (vii) TBL3_HUMAN or a fragment, variant or homologue thereof; (viii) WDR36_HUMAN or a fragment, variant or homologue thereof; and (ix) NOC4L_HUMAN or a fragment, variant or homologue thereof.

2. The protein complex of claim 1 wherein the polypeptide components, or fragments, variants or homologues thereof, are mammalian.

3. The protein complex of claim 1 wherein the polypeptide components, or fragments, variants or homologues thereof, are human.

4. The protein complex of claim 1 wherein the polypeptide components have the amino acid sequence provided in SEQ ID NOs: 1 to 9.

5. The protein complex of claim 1 wherein the polypeptide components, or fragments, variants or homologues thereof, are yeast.

6. The protein complex of claim 1 wherein the complex comprising polypeptide components: (i) YBA4_YEAST or a fragment, variant or homologue thereof; (ii) PWP_YEAST or a fragment, variant or homologue thereof; (iii) UTP7_YEAST or a fragment, variant or homologue thereof; (iv) UTP18_YEAST or a fragment, variant or homologue thereof; (v) MPP10_YEAST or a fragment, variant or homologue thereof; (vi) DIP2_YEAST or a fragment, variant or homologue thereof; (vii) UTP13_YEAST or a fragment, variant or homologue thereof; (viii) YL409_YEAST or a fragment, variant or homologue thereof; (ix) NOC4_YEAST or a fragment, variant or homologue thereof; and (x) UTP6 YEAST or a fragment, variant or homologue thereof.

7. The protein complex of claim 1 wherein the polypeptide components have the amino acid sequence provided in SEQ ID NOs: 10 to 19.

8. The protein complex of claim 1 wherein at least one of the polypeptide components further comprise a fusion tag or label.

9-29. (canceled)

30. A method of assessing whether a subject has or is likely to develop an eye disorder comprising determining whether the subject has an altered amount, function, activity, composition and/or formation of a protein complex according to claim 1.

31. The method of claim 30 wherein if the subject has an elevated amount, function, activity, and/or formation of the protein complex, then this indicates that the subject has or is likely to develop an eye disorder.

32. The method of claim 30 wherein if the subject has a reduced amount, function, activity, and/or formation of the protein complex, then this indicates that the subject has or is likely to develop an eye disorder.

33. The method of claim 30 wherein if the subject has an altered composition of the protein complex, then this indicates that the subject has or is likely to develop an eye disorder.

34. The method of claim 30 wherein if the subject has one or more of the following mutations: TBL3, nt 3895 G>A; UTP20, nt 10156 A>C; UTP20, nt 73119 T>C; WDR36, nt 191 T>C; WDR36, nt 6579 C>T; WDR36, nt 17980 G>A; PWP2, nt 14867 T>A; WDR3, nt 2019 T>G, then this indicates that the subject has or is likely to develop an eye disorder.

35. The method of claim 30 wherein if the subject has one or more of the following mutations: WDR36, amino acid change L25P; WDR36, amino acid change A163V; PWP2, amino acid change F551I, then this indicates that the subject has or is likely to develop an eye disorder.

36. The method of claim 30 wherein the subject is a mammalian subject.

37. The method of claim 30 wherein the eye disorder is glaucoma.

38-43. (canceled)

44. A kit for assessing whether a subject has or is likely to develop an eye disorder comprising means for determining the amount, function, activity, composition and/or formation of a protein complex according to claim 1.

45. The kit of claim 44 wherein the eye disorder is glaucoma.

46. The protein complex of claim 1 wherein the polypeptide components have at least 70% sequence identity with the amino acid sequence provided in SEQ ID NOs: 1 to 9.

47. The protein complex of claim 1 wherein the polypeptide components have at least 70% sequence identity with the amino acid sequences provided in SEQ ID NOs: 10 to 19.
